(19) United States
(12) Patent Application Publication
(10) Pub. No.: US 2003/0195706 A1
(43) Pub. Date: Oct. 16, 2003

(54) Method for classifying genetic data

(76) Inventor: Michael Korenberg, Battersea (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/428,776

(22) Filed: May 5, 2003

Related U.S. Application Data
(63) Continuation-in-part of application No. PCT/CA01/01547, filed on Nov. 5, 2001.
(60) Provisional application No. 60/391,597, filed on Jun. 27, 2002.

(30) Foreign Application Priority Data
Nov. 20, 2000 (CA) 2,325,225

(51) Int. Cl. G01N 33/48

(52) U.S. Cl. 702/19

(57) ABSTRACT

The present invention provides a method for class prediction in bioinformatics based on identifying a nonlinear system that has been defined for carrying out a given classification task. Information characteristic of exemplars from the classes to be distinguished is used to create training inputs, and the training outputs are representative of the class distinctions to be made. Nonlinear systems are found to approximate the defined input/output relations, and these nonlinear systems are then used to classify new data samples. In another aspect of the invention, information characteristic of exemplars from one class are used to create a training input and output. A nonlinear system is found to approximate the created input/output relation and thus represent the class, and together with nonlinear systems found to represent the other classes, is used to classify new data samples.

The present application claims priority from U.S. provisional applications No. 60/245,236, entitled “Use of Fast Orthogonal Search and Other Model-Building Techniques for Interpretation of Gene Expression Profiles”, filed Nov. 3, 2000, No. 60/249,462, entitled “Nonlinear System Identification for Class Prediction in Bioinformatics and Related Applications”, filed Nov. 20, 2000, No. 60/268,019, entitled “Prediction of Clinical Outcome Using Gene Expression Profiles”, filed Feb. 13, 2001, No. 60/391,597 entitled “A Decision Criterion Based on the Responses of A Trained Model to Additional Exemplars of the Classes” filed Jun. 27, 2002, Canadian Patent Application No. 2,325,225, entitled “Nonlinear System Identification for Class Prediction in Bioinformatics and Related Applications”, filed Nov. 20, 2000, and PCT patent application PCT/CA01/01547, entitled “Nonlinear System Identification for Class Prediction in Bioinformatics and related applications” filed Nov. 5, 2001, which applications are incorporated herein by this reference.

### TECHNICAL FIELD

The present invention pertains to a method for class prediction in bioinformatics based on identifying a nonlinear system that has been defined for carrying out a given classification task.

### BACKGROUND OF THE INVENTION

Many areas in bioinformatics involve class prediction, for example: (1) assigning gene expression patterns or profiles to defined classes, such as tumor and normal classes; (2) recognition of active sites, such as phosphorylation and ATP-binding sites, on proteins; (3) predicting whether a molecule will exhibit biological activity, e.g., in drug discovery, including the screening of databases of small molecules to identify molecules of possible pharmaceutical use; (4) distinguishing exon from intron DNA and RNA sequences, and determining their boundaries; and (5) establishing genotype/phenotype correlations, for example to optimize cancer treatment, or to predict clinical outcome or various neuromuscular disorders.

A recent paper by Golub et al. (1999), “Molecular Classification of Cancer: Class Discovery and Class Prediction by Gene Expression Monitoring”, in Science, vol. 286, pp. 531-537, describes a method for predicting the class of tissue samples based on simultaneous monitoring of expression levels of thousands of genes via oligonucleotide microarrays, and is incorporated herein by this reference. Each tissue sample is represented by a gene expression pattern, often called a gene expression profile, a set of quantitative expression levels of the selected genes. The Golub et al. (1999) method is statistically-based and requires a number of profiles known to belong to each of the classes to be distinguished. A voting scheme is set up based on a subset of “informative genes” and each new tissue sample is classified based on a vote total, provided that a “prediction strength” measure exceeds a predetermined threshold. When the prediction strength is low, the class of the sample is uncertain, and resort must be made to other methods.

Accordingly, there is a need for an improved method of predicting the class of a genetic sample when there are only a few known examples (exemplars) of the classes to be distinguished. The revised method preferably uses little training data to build a finite-dimensional nonlinear system that then acts as a class predictor. The class predictor can be combined with other predictors to enhance classification accuracy, or the created class predictor can be used to classify samples when the classification by other predictors is uncertain.

### SUMMARY OF THE INVENTION

The present invention provides a method for class prediction in bioinformatics based on identifying a nonlinear system that has been defined for carrying out a given classification task. Information characteristic of exemplars from the classes to be distinguished is used to create training inputs, and the training outputs are representative of the class distinctions to be made. Nonlinear systems are found to approximate the defined input/output relations, and these nonlinear systems are then used to classify new data samples. In another aspect of the invention, information characteristic of exemplars from one class are used to create a training input and output. A nonlinear system is found to approximate the created input/output relation and thus represent the class, and together with nonlinear systems found to represent the other classes, is used to classify new data samples.

In accordance with a preferred embodiment of the present invention, there is provided a method for constructing a class predictor in the area of bioinformatics. The method includes the steps of selecting information characteristic of exemplars from the families (or classes) to be distinguished, constructing a training input with segments containing the selected information for each of the families, defining a training output to have a different value over segments corresponding to different families, and finding a system that will approximate the created input/output relation. In accordance with an embodiment of the invention, the characterizing information may be the expression levels of genes in gene expression profiles, and the families to be distinguished may represent normal and various diseased states.

In accordance with another aspect of the present invention, there is provided a method for using fast orthogonal search or other model-building techniques to select genes whose expression levels can be used to distinguish between different families.

In accordance with yet another aspect of the present invention, there is provided a method for classifying protein sequences into structure/function groups, which can be used for example to recognize active sites on proteins, and the characterizing information may be representative of the primary amino acid sequence of a protein or a motif.

In accordance with yet another aspect of the present invention, there is provided a method for predicting whether a molecule will exhibit biological activity, e.g., in drug discovery, including the screening of databases of small molecules to identify molecules of possible pharmaceutical use. In this case the characterizing information may represent properties such as molecular shape, the electrostatic vector fields of small molecules, molecular weight, and the number of aromatic rings, rotatable bonds, hydrogen-bond donor atoms and hydrogen-bond acceptor atoms.

In accordance with yet another aspect of the present invention, there is provided a method for distinguishing between exon and intron DNA and RNA sequences, and for determining their boundaries. In this case the characterizing information may represent a sequence of nucleotide bases on a given strand.

In accordance with yet another aspect of the present invention, there is provided a method for finding genotype/phenotype relationships and correlations. In this case, the characterizing information may represent factors such as pathogenic mutation, polymorphic allelic variants, epigenetic modification, and SNPs (single nucleotide polymorphisms), and the families may be various human disorders, e.g., neuromuscular disorders.

In accordance with yet another aspect of the present invention, there is provided a method for constructing a class predictor in the area of bioinformatics by combining the predictors described herein with other predictors.

The invention is further summarized in Appendix D, which also includes additional detailed embodiments of the invention.

### BRIEF DESCRIPTION OF THE FIGURES

The present invention will now be described, by way of example only, with reference to the drawings in which:

FIG. 1 illustrates the form of the parallel cascade model used in classifying the gene expression profiles, proteomics data, and the protein sequences. Each L is a dynamic linear element, and each N is a polynomial static nonlinearity;

FIG. 2 shows the training input x(i) formed by splicing together the raw expression levels of genes from a first ALL profile #1 and a first AML profile #28. The genes used were the 200 having greatest difference in expression levels between the two profiles. The expression levels were appended in the same relative ordering that they had in the profile;

FIG. 3 shows the training output y(i) (solid line) defined as −1 over the ALL portion of the training input and 1 over the AML portion, while the dashed line represents calculated output z(i) when the identified parallel cascade model is stimulated by training input x(i);

FIG. 4A shows the training input x(i) formed by splicing together the raw expression levels of genes from the first “failed treatment” profile #28 and first “successful treatment” profile #34; the genes used were the 200 having greatest difference in expression levels between the two profiles;

FIG. 4B shows that the order used to append the expression levels of the 200 genes caused the auto-covariance of the training input to be nearly a delta function; indicating that the training input was approximately white;

FIG. 4C shows the training output y(i) (solid line) defined as −1 over the “failed treatment” portion of the training input and 1 over the “successful treatment” portion; the dashed line represents calculated output z(i) when the identified model is stimulated by training input x(i);

FIG. 5A shows the impulse response functions of linear elements L₂ (solid line), L₄ (dashed line), L₆ (dotted line) in the 2^{nd}, 4^{th}, 6^{th} cascades of the identified model;

FIG. 5B shows the corresponding polynomial static nonlinearities N₂ (diamonds), N₄ (squares), and N₆ (circles) in the identified model.

### DETAILED DESCRIPTION OF SPECIFIC EMBODIMENTS

(A) Use of Parallel Cascade Identification to Predict Class of Gene Expression Profiles

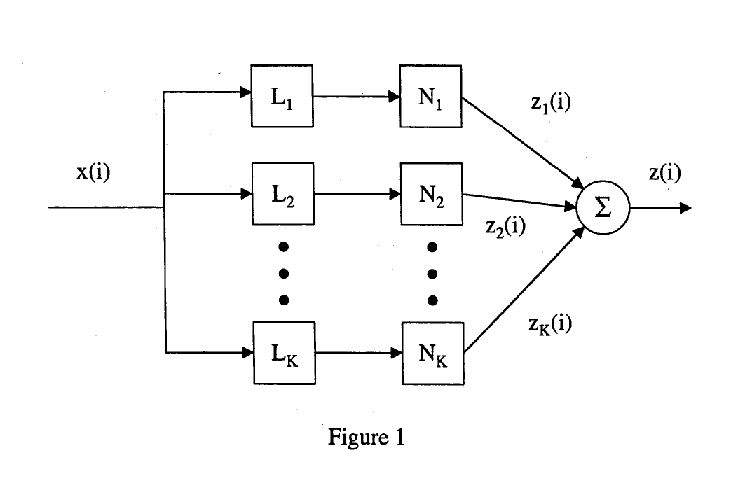

In a first embodiment of the present invention, one or more representative profiles, or portions of profiles, from the families to be distinguished are concatenated (spliced) in order to form a training input. The corresponding training output is defined to have a different value over input segments from different families. The nonlinear system having the defined input/output relation would function as a classifier, and at least be able to distinguish between the training representatives (i.e., the exemplars) from the different families. A parallel cascade or other model is then found to approximate this nonlinear system. While the parallel cascade model is considered here, the invention is not limited to use of this model, and many other nonlinear models, such as Volterra functional expansions, and radial basis function expansions, can instead be employed. The parallel cascade model used here (FIG. 1) comprises a sum of cascades of dynamic linear and static nonlinear elements. “Dynamic” signifies that the element possesses memory: its output at a given instant i depends not only upon its input x at instant i but upon past input values at instants i−1, . . . , i−R (memory length=R+1). Every nonlinear element is a polynomial, so that each cascade output zₖ(i), and hence the overall model output z(i), reflect high-order nonlinear interactions of gene expression values. For generality of approximation, it suffices if each cascade comprises a dynamic linear element followed by a static nonlinearity (as in FIG. 1), but additional alternating dynamic linear and static nonlinear elements could optionally be inserted in any path: see Korenberg (1991) “Parallel Cascade Identification and Kernel Estimation for Nonlinear Systems”, in Annals of Biomedical Engineering, vol. 19, pp.429-455, which is incorporated herein by reference.

The memory length of the nonlinear model may be taken to be considerably shorter than the length of the individual segments that are spliced together to form the training input. Suppose that x(i) is the input, and y(i) the output, of a discrete-time nonlinear system, where i=1, . . . , I. If y(i) depends only upon the input values x(i), x(i−1), . . . , x(i−R), i.e., back to some maximum finite delay R, then the system is said to have a finite memory of length (R+1). (Alternatively, it could be said that the memory length is only R, because for a system with no memory the output y at instant i depends only upon the input x at that same instant.) If the assumed memory length for the model to be identified is shorter than the individual segments of the training input, the result is to increase the number of training examples. This is explained here in reference to using a single exemplar from each of two families to form the training input, but the same principle applies when more representatives from several families are spliced together to create the input. Note that, in the case of gene expression profiles, the input values will represent gene expression levels, however it is frequently convenient to think of the input and output as time-series data.

### EXAMPLE 1

Consider distinguishing between profiles from two classes, acute lymphoblastic leukemia (ALL) and acute myeloid leukemia (AML), as in the paper by Golub et al. (1999). Each of their profiles contained the expression levels of 6817 human genes, but because of duplicates and additional probes in the Affymetrix microarray, in total 7129 gene expression levels were present in each profile. One simple approach is to simply splice together entire 7129 point profiles from the two families to form the training input. However, many or most of the genes in an entire profile might not be relevant to distinguishing between ALL and AML classes, so use of entire profiles could make the training of effective parallel cascade models more difficult. Moreover, the Golub et al. (1999) paper teaches the importance of finding “informative genes . . . most closely correlated with ALL-AML distinction in the known samples”.

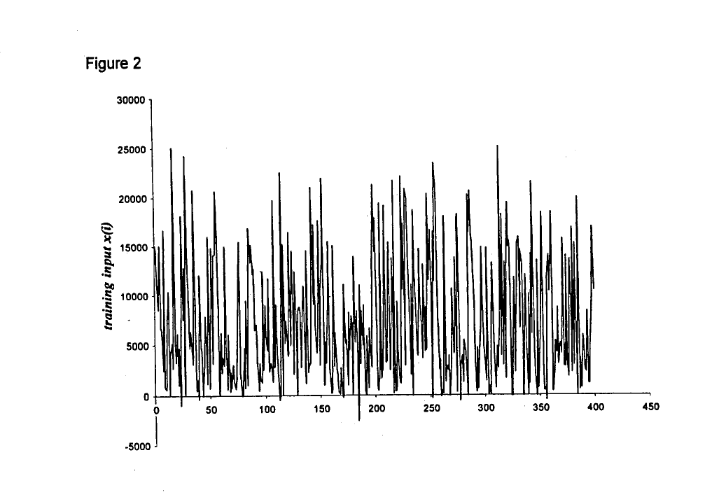

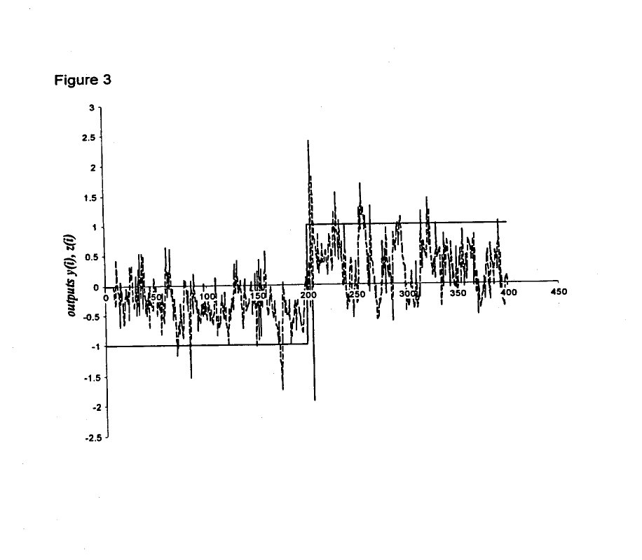

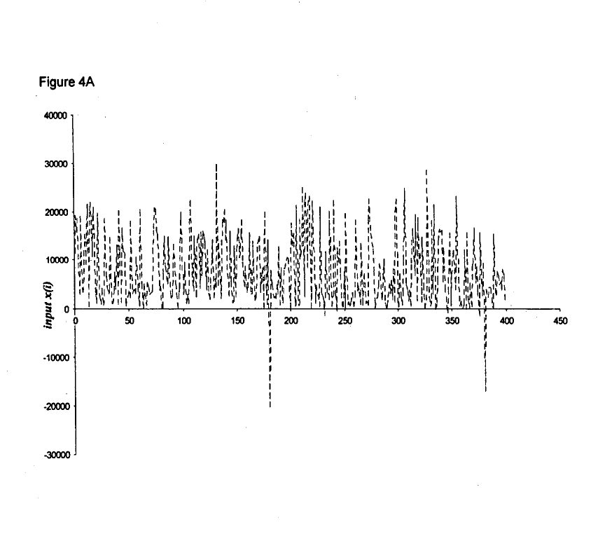

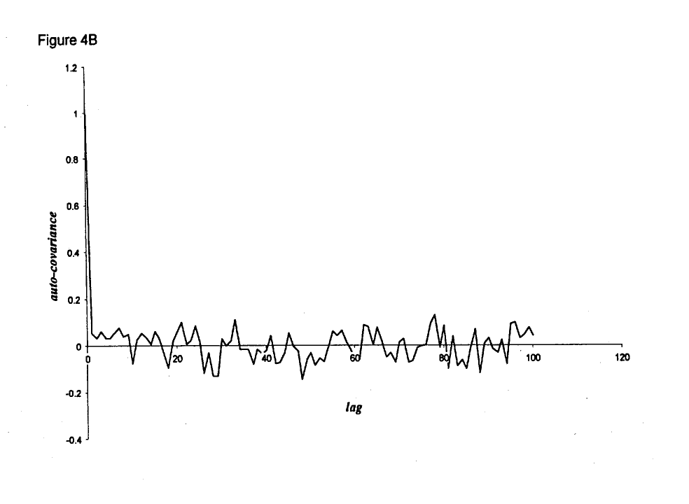

Therefore, the first ALL profile (#1 of Golub et al. training data) and the first AML profile (#28 of their training data) were compared and 200 genes that exhibited that largest absolute difference in expression levels were located. In another embodiment of the present invention, a different number of genes may be located and used. For each of these genes, the absolute value of the difference between the corresponding raw scores on profiles #1 and 28 ranked in the top 200 of the 7129 genes. The raw expression values for these 200 genes were juxtaposed to form the ALL segment to be used for training, and the AML segment was similarly prepared. The 200 expression values were appended in the same relative order that they had in the original profile, and this is true for all the examples described in this patent application. However, it has been found that other ordering schemes may be beneficial, for example those that cause the autocovariance of the training input to be almost a delta (i.e., discrete impulse) function. This is discussed further in the section concerned with predicting treatment response and in the discussion of STEPS that follow Example 5. Each time the same order was used in juxtaposing the expression values to prepare a segment. The two information-rich segments were then spliced together to form a 400 point training input x(i) (FIG. 2), and the corresponding output y(i) (FIG. 3, solid line) was defined to be −1 over the ALL segment, and 1 over the AML segment.

A parallel cascade model was then identified with a memory length (R+1)=10, and 5^{th} degree polynomial static nonlinearities, with 19 cascade paths in total. The values of 200 for the segment length and 10 for the memory length were chosen because Golub et al. (1999) reported that between 10 and 200 informative genes could be used in their procedure with excellent results (see further discussion below).

A 5^{th} degree polynomial was chosen for each static nonlinearity because that was the smallest degree found effective in a recent protein sequence classification application (Korenberg et al., 2000a, “Parallel Cascade Identification as a Means for Automatically Classifying Protein Sequences into Structure/Function Groups”, vol. 82, pp. 15-21, which is incorporated herein by reference, attached hereto as Appendix A). Further details about parallel cascade identification are given below in the section involving protein sequence classification, and in Korenberg (1991).

Note that in finding the impulse responses of the dynamic linear elements in FIG. 1, the procedure in Korenberg (1991) creates candidate impulse response functions, involving random elements which can be controlled by a random number generator. (A threshold test is used to decide which candidate cascades will be accepted into the model.) In all examples in this application, the same random number sequence was used every time, so that differences in classification due to a change in model parameters such as memory length or polynomial degree would be readily evident. Alternatively, if different random sequences are used in different trials of parameter values, then several models should be obtained for each possible choice of parameter values. In this way, changes in classification accuracy due to changes in parameter settings, rather than changes in the random sequence used, will become evident. Alternatively, an appropriate logical deterministic sequence, rather than a random sequence, can be used in creating candidate impulse responses: see Korenberg et al. (2001) “Parallel cascade identification and its application to protein family prediction”, J. Biotechnol., Vol. 91, 35-47, which is incorporated herein by this reference.

A maximum of 20 cascade paths was allowed to ensure that the number of variables introduced into the parallel cascade model was significantly less than the number of output points used in the identification, as also discussed in Korenberg et al. (2000a). The cascade paths were selected using a criterion (Korenberg, 1991) based on a standard correlation test. The criterion requires specification of a threshold value T (see Eq. (11) of Korenberg et al., 2000b, “Automatic Classification of Protein Sequences into Structure/Function Groups via Parallel Cascade Identification: A Feasibility Study”, Annals of Biomedical Engineering, vol. 28, pp. 803-811, which is incorporated herein by this reference, attached hereto as Appendix B), here set equal to 6. This value was chosen so that very nearly the maximum number (20) of cascade paths was selected out of 2000 candidate cascades. There is no special virtue in setting 2000 for the number of candidates, and some other large number could be used instead. Above the parameter values for memory length, polynomial degree, maximum number of cascades in the model, and threshold T were chosen from other work in bioinformatics. However, the choices could also be made by testing the effectiveness of trial values over a small verification set (Korenberg et al., 2000a) and this is preferable when the amount of data permits, and is illustrated in other examples below.

In this example, the identified model had a mean-square error (MSE) of 65.11%, expressed relative to the variance of the output signal. FIG. 3 shows that when the training input x(i) was fed through the identified parallel cascade model, the resulting output z(i) (dashed line) is predominately negative over the ALL segment, and positive over the AML segment, of the input. Only portions of the first ALL and the first AML profiles had been used to form the training input. The identified parallel cascade model was then tested on classifying the remaining ALL and AML profiles in the first set used for training by Golub et al. (1999).

To classify a profile, the expression levels corresponding to the genes selected above are appended in the same order as used above to form a segment for input into the identified parallel cascade model, and the resulting model output is obtained. If the mean of the model output is less than zero, the profile is assigned to the ALL class, and otherwise to the AML class. In calculating the mean output, the averaging preferably begins on the (R+1)-th point, since this is the first output point obtained with all necessary delayed input values known. Other classification criteria, for example based on comparing two MSE ratios (Korenberg et al., 2000b), could also be employed.

The classifier correctly classified 19 (73%) of the remaining 26 ALL profiles, and 8 (80%) of the remaining 10 AML profiles in the first Golub et al. set. The classifier was then tested on an additional collection of 20 ALL and 14 AML profiles, which included a much broader range of samples. Over this second set, the parallel cascade model correctly classified 15 (75%) of the ALL and 9 (64%) of the AML profiles. No normalization or scaling was used to correct expression levels in the test sequences prior to classification. It is important to realize that these results were obtained after training with an input created using only the first ALL and first AML profiles in the first set.

Means and standard deviations for the training set are used by Golub et al. in normalizing the log expression levels of genes in a new sample whose class is to be predicted. Such normalization may have been particularly important for their successfully classifying the second set of profiles which Golub et al. (1999) describe as including “a much broader range of samples” than in the first set. Since only one training profile from each class was used to create the training input for identifying the parallel cascade model, normalization was not tried here based on such a small number of training samples. Scaling of each profile, for example by dividing each expression level by the mean of all levels has been recommended (see, for example, Alon et al., 1999, “Broad Patterns of Gene Expression Revealed by Clustering Analysis of Tumor and Normal Colon Tissues Probed by Oligonucleotide Arrays”, Proc. Natl. Acad. Sci. USA, vol. 96, pp. 6745-6750, Cell Biology, which is incorporated herein by reference). Such scaling is a way of compensating for variations between profiles (Alon et al., 1999). Alternatively, each expression value can be divided by the root-mean-square of all the expression levels in the profile. Other forms of scaling will be well-known to persons skilled in the art. However, no scaling of the data obtained from the Golub et al web site (Datasets: http://www-genome.wi.mit.edu/MPR/data_set_ALL_AML.html) was used here, or below where accurate classification is achieved of the second, set of profiles in Golub et al. (1999). It should be noted that Golub et al. had already re-scaled the intensity values so that overall intensities for each chip were equivalent.

The above parallel cascade classification results were obtained using raw gene expression levels. The results set out above may be slightly improved by instead using the log of the expression level, to the base 10. Before the log is taken, any negative or very small raw expression value is increased to 100. When this was done in the method of present invention, before identifying the parallel cascade model, on the same two training profiles used previously, 20 (77%) of 26 ALL and 9 (90%) of 10 AML profiles were correctly classified. Again the parallel cascade had a memory length of 10, and 5^{th} degree polynomial static nonlinearities. A higher threshold T=10 was used, only 7 cascade paths were selected, and the resulting MSE was 80.67%.

### EXAMPLE 2

Use of Multiple Training Exemplars with Raw Expression Levels

The above results for parallel cascade classification of gene expression profiles were obtained from using 200 genes' expression levels of one profile from each class to construct the training input. In contrast, the results reported by Golub et al. (1999) were for training with all, or all but one, of the 27 ALL and 11 AML exemplars in their first set. All but one exemplar in the first set were used for training when they predicted the class of the omitted profile in the same set (cross-validation), repeating this procedure until each of the profiles had been tested. All the exemplars were used for training when they predicted the class of profiles in the second set. Parallel cascade classifiers will not always be successful if the training input is based on only a single example of each of the classes to be distinguished. The single examples must be representative of other class members.

Multiple 200-point examples from each class can be appended to form a more information-rich training input, with the output again defined to have a different value over each segment from a different class. Suppose again that the nonlinear system to be identified has a memory of length (R+1), i.e., its output y(i) depends only upon x(i), . . . ,x(i−R). Then in carrying out the system identification, we preferably exclude from calculation the first R points of each segment joined to form the input (Korenberg et al., 2000 a, b). This avoids introducing error due to the transition regions where the segments are joined together.

To continue the present example, the first 11 of the 27 ALL profiles in the first set of Golub et al. (1999) were each used to extract a 200-point segment characteristic of the ALL class. The first 5 profiles (i.e., #28-#32) of the 11 AML profiles in the first set were similarly used, but in order to extract 11 200-point segments, these profiles were repeated in sequence #28-#32, #28-#32, #28. The 200 expression values were selected as follows. For each gene, the mean of its raw expression values was computed over the 11 ALL profiles, and the mean was also computed over the 11 AML profiles (which had several repeats). Then the absolute value of the difference between the two means was computed for the gene. The 200 genes having the largest of such absolute values were selected. The 11 ALL and 11 AML segments were concatenated to form the training input, and the training output was again defined to be −1 over each ALL segment and 1 over each AML segment.

Because there actually were 16 different 200-point segments, the increased amount of training data enabled many more cascades to be used in the model. To provide significant redundancy (more output points used in the identification than variables introduced in the parallel cascade model), a limit of 100 cascades was set for the model. It was found that if each polynomial in the cascade had a degree of 9, and if a threshold T=12 was used, then nearly 100 cascades would be accepted out of 2000 candidates. Again, a memory length of 10 was used for each of the dynamic linear elements in the cascades. The identified model had a MSE of 62.72%. Since 11 ALL and 5 AML profiles had been used in constructing the training input, 16 ALL and 6 AML profiles remained for testing. The identified model correctly recognized 15 (93.8%) of the ALL and 5 (83.3%) of the AML test profiles. The model flawlessly classified the ALL and AML profiles used in constructing the training input.

The above examples may be briefly summarized in the following steps:

Step 1—Compare the gene expression levels in the training profiles and select a set of genes that assist in distinguishing between the classes.

Step 2—Append the expression levels of selected genes from a given profile to produce a segment representative of the class of that profile. Repeat for each profile, maintaining the same order of appending the expression levels.

Step 3—Concatenate the representative segments to form a training input.

Step 4—Define an input/output relation by creating a training output having values corresponding to the input values, where the output has a different value over each representative segment from a different class.

Step 5—Identify a parallel cascade model (FIG. 1) to approximate the input/output relation.

Step 6—Classify a new gene expression profile by (a) appending the expression levels of the same genes selected above, in the same order as above, to produce a segment for input into the identified parallel cascade model; (b) apply the segment to the parallel cascade model and obtain the corresponding output; and (c) if the mean of the parallel cascade output is less than zero, then assign the profile to the first class, and otherwise to the second class.

### EXAMPLE 3

Use of Multiple Training Exemplars with Log Expression Levels

Almost all of the above results were obtained using raw gene expression levels. However, as noted above, in place of the raw expression level, the logarithm of the expression level, to the base 10. Before the log is taken, any negative or very small raw value is increased to 100. Results are provided below for use of multiple training exemplars from each class with the log of the expression level.

The first 15 ALL profiles (#1-#15 of Golub et al. first data set) were each used to extract a 200-point segment characteristic of the ALL class, as described immediately below. Since there were only 11 distinct AML profiles in the first Golub et al. set, the first 4 of these profiles were repeated, to obtain 15 profiles, in sequence #28-#38, #28-#31. For each gene, the mean of its raw expression values was computed over the 15 ALL profiles, and the mean was also computed over the 15 AML profiles. Then the absolute value of the difference between the two means was computed for the gene. The 200 genes having the largest of such absolute values were selected. This selection scheme is similar to that used in Golub et al. (1999) but in the present application, selection was made using raw expression values and not their logs, and the difference in the means was not measured relative to standard deviations within the classes. However, once a gene was selected, the log of its expression level was used, rather than the raw value, in forming the representative segment for each profile.

The 15 ALL and 15 AML segments were concatenated to form the training input, and the training output was defined to be −1 over each ALL segment and 1 over each AML segment. Because there actually were 26 different 200-point segments, the increased amount of training data enabled many more cascades to be used in the model, as compared to the use of one representative segment from each class. To have significant redundancy (more output points used in the identification than variables introduced in the parallel cascade model), a limit of 200 cascades was set for the model. Note that not all the variables introduced into the parallel cascade model are independent of each other. For example, the constant terms in the polynomial static nonlinearities can be replaced by a single constant. However, to prevent over-fitting the model, it is convenient to place a limit on the total number of variables introduced, since this is an upper bound on the number of independent variables.

In Example 1, when a single representative segment from each of the ALL and AML classes had been used to form the training input, the parallel cascade model to be identified was assumed to have a memory length of 10, and 5^{th} degree polynomial static nonlinearities. When log of the expression level was used instead of the raw expression level, the threshold T was set equal to 10. These parameter values are now used here, when multiple representative segments from each class are used in the training input with log expression levels rather than the raw values.

If the assumed memory length of the model is (R+1), then as noted above, in carrying out the identification, those output points corresponding to the first R points of each segment joined to form the training input are preferably excluded from computation. Since there were 26 different 200-point segments, and since the assumed memory length was 10, the number of output points used in the identification was considered (for the present purpose of preventing over-fitting) to be 26×(200-9)=4966. Since each cascade in the model will have a dynamic linear element with memory length 10, and a polynomial static nonlinearity of degree 5, then for a maximum of 200 cascades, the number of variables introduced will be at most 200×(10+5+1)=3200. This is significantly less than the number of output points to be used in the identification, and avoids over-fitting the model.

The identified parallel cascade model had a mean-square error of about 71.3%, expressed relative to the variance of the output signal. While a limit of 200 cascades had been set, in fact, with the threshold T=10, only 100 cascades were selected out of 2000 candidates. There is no special virtue in setting 2000 for the number of candidates, and some other large number could be used instead.

Classification Results for ALL vs AML

The representative 200-point segments for constructing the training input had come from the first 15 of the 27 ALL profiles, and all 11 of the AML profiles, in the first data set from Golub et al. (1999). The performance of the identified parallel cascade model was first investigated over this data set, using two different decision criteria.

For each profile to be classified, the log of the expression levels corresponding to the 200 genes selected above are appended, in the same order as used above, to form a segment for input into the identified parallel cascade model. The resulting model output z(i), i=0, . . . ,199, is obtained.

The first decision criterion examined has already been used above, namely the sign of the mean output. Thus, if the mean of the model output was negative, the profile was assigned to the ALL class, and if positive to the AML class. In calculating the mean output, the averaging began on the 10^{th} point, since this was the first output point obtained with all necessary delayed input values known.

The second decision criterion investigated is based on comparing two MSE ratios and is mentioned in the provisional application (Korenberg, 2000a).

This criterion compares the MSE of the model output z(i) from −1, relative to the corresponding MSE over the ALL training segments, with the MSE of z(i) from 1, relative to the MSE over the AML training segments.

In more detail, the first ratio, r₁, is

$$\frac{\overline{{( z ( i ) + 1 )}^{2}}}{{e}_{1}}$$

where the overbar denotes the mean (average over i), and e₁ is the MSE of the model output from −1 over the ALL training segments. The second ratio, r₂, is

$$\frac{\overline{{( z ( i ) - 1 )}^{2}}}{{e}_{2}}$$

where e₂ is the MSE of the model output from 1 over the AML training segments. Each time an MSE is computed, the averaging begins on the 10^{th} point, since the model has a memory length of 10 for this classification task. If r₁ is less than r₂, then the profile is classified as ALL, and if greater, as AML.

When the two decision criteria were compared over the 27 ALL and 11 AML profiles in the first Golub et al. (1999) data set, the second criterion, based on comparing MSE ratios, resulted in higher classification accuracy.

Hence the latter criterion was chosen to be used in class prediction over the second (independent) data set from Golub et al. (1999) that included a much broader range of samples. The parallel cascade model correctly classified all 20 of the ALL profiles, and 10 of the 14 AML profiles, in the second set.

In order to ensure that the successful classification over this set was not a fortuitous result of reusing the threshold value T=10 from Example 1, model identification and testing were repeated for a variety of different threshold values. Each time the same training input, which used multiple representative segments from each class, was employed. The threshold T was successively set equal to each integer value from 5 to 11, with memory length fixed at 10, and polynomial degree at 5. The second decision criterion, based on MSE ratios, was used. The resulting parallel cascade models all had identical accuracy over the first data set, making one error on the 27 ALL and one error on the 11 AML profiles.

On the second data set, all the models successfully classified all 20 of the ALL profiles. Of the 14 AML profiles, the models' performance ranged from 8-12 correct classifications. The poorest score of 6 errors, for T=11, came from the model with the largest mean-square error when identified, about 73.3%, having 83 cascades. The best score of 2 errors, for T=7, came from a model with a mean-square error of about 68.7%, having 137 cascades. In summary, all the models trained using 15 ALL and 11 AML profiles from the first Golub et al. data set made correct class predictions on the second data set except for 2-6 profiles. The model for threshold T=7 stood out as the most robust as it had the best performance over the first data set using both decision criteria (sign of mean output, and comparing MSE ratios) of values nearest the middle of the effective range for this threshold. More importantly, the above accuracy results from using a single classifier. As shown in the section dealing with use of fast orthogonal search and other model-building techniques, accuracy can be significantly enhanced by dividing the training profiles into subsets, identifying models for the different subsets, and then using the models together to make the classification decision. This principle can also be used with parallel cascade models to increase classification accuracy.

Discussion of the Above Example

The described nonlinear system identification approach utilizes little training data. This method works because the system output value depends only upon the present and a finite number of delayed input (and possibly output) values, covering a shorter length than the length of the individual segments joined to form the training input. This requirement is always met by a model having finite memory less than the segment lengths, but applies more generally to finite dimensional systems. These systems include difference equation models, which have fading rather than finite memory. However, the output at a particular “instant” depends only upon delayed values of the output, and present and delayed values of the input, covering a finite interval. For example the difference equation might have the form:

y(i)=F[y(i−1), . . . ,y(i−l₁),x(i), . . . ,x(i−l₂)]

So long as the maximum of the output delay l₁ and the input delay l₂ is less than the number of points in each input segment, we derive multiple training examples from each segment joined to form the input.

To illustrate, the parallel cascade model was assumed above to have a memory length of 10 points, whereas the ALL and AML segments each comprised 200 points. Having a memory length of 10 means that we assume it is possible for the parallel cascade model to decide whether a segment portion is ALL or AML based on the expression values of 10 genes. Thus the first ALL training example for the parallel cascade model is provided by the first 10 points of the ALL segment, the second ALL training example is formed by points 2 to 11, and so on. Hence each 200-point segment actually provides 191 training examples, in total 382 training examples, and not just two, provided by the single ALL and AML input segments. Why was each segment taken to be 200 points, and the memory length 10 points? This was because, as noted above, the Golub et al. (1999) article reported that extremely effective predictors could be made using from 10 to 200 genes. In other embodiments of the present invention, a different number of points may be used for each segment or a different memory length, or both, may be used.

Each training exemplar can be usefully fragmented into multiple training portions, provided that it is possible to make a classification decision based on a fragmented portion. Of course the fragments are overlapping and highly correlated, but the present method gains through training with a large number of them, rather than from using the entire exemplar as a single training example. This use of fragmenting of the input segments into multiple training examples results naturally from setting up the classification problem as identifying a finite dimensional nonlinear model given a defined stretch of input and output data. However, the principle applies more broadly, for example to nearest neighbor classifiers. Thus, suppose we were given several 200-point segments from two classes to be distinguished. Rather than using each 200-point segment as one exemplar of the relevant class, we can create 191 10-point exemplars from each segment.

Moreover, the use of fragmenting enables nearest neighbor methods as well as other methods such as linear discriminant analysis, which normally require the class exemplars to have equal length, to work conveniently without this requirement. Thus, even if some of the original exemplars have more or less than, e.g., 200 points, they will still be fragmented into, e.g., 10-point portions that serve as class examples. To classify a query profile or other sequence, it is first fragmented into the overlapping 10-point portions. Then a test of similarity (e.g. based on a metric such as Euclidean distance) is applied to these fragmented portions to determine the membership of the query sequence.

Note that setting up the class predictor problem as the identification of a nonlinear system, with a memory length less than the shortest of the segments joined to form the training input, is a different approach from training an artificial neural network to predict class. In the latter case, the neural network is trained by simply presenting it with numerous examples of class members. There is no fragmentation of the exemplars to create multiple training examples from each using the sliding window approach described above for the case of system identification. As already noted, this fragmentation occurs naturally from having the nonlinear system's memory length shorter than the segments joined to form the training input. In addition, while we have considered the nonlinear system to be causal (i.e., its output can depend on present and lagged input values, but not advanced values) this is not essential, and systems also having finite anticipation can be considered for the classifier.

To identify genes that effectively distinguish between the classes, the absolute difference in either the raw expression level or the log of the expression level may be used, as described above. Alternatively, clustering of genes using the method of Alon et al. (1999) “reveals groups of genes whose expression is correlated across tissue types”. The latter authors also showed that “clustering distinguishes tumor and normal samples even when the genes used have a small average difference between tumor and normal samples”. Hence clustering may also be used to find a group of genes that effectively distinguishes between the classes.

Alternatively, fast orthogonal search (Korenberg, 1989a, “A Robust Orthogonal Algorithm for System Identification and Time Series Analysis”, in Biological Cybernetics, vol. 60, pp. 267-276; Korenberg 1989b, “Fast Orthogonal Algorithms for Nonlinear System Identification and Time-Series Analysis”, in Advanced Methods of Physiological System Modeling, vol. 2, edited by V. Z. Marmarelis, Los Angeles: Biomedical Simulations Resource, pp. 165-177, both of which are incorporated herein by reference), iterative fast orthogonal search (Adeney and Korenberg, 1994, “Fast Orthogonal Search for Array Processing and Spectrum Estimation”, IEE Proc. Vis. Image Signal Process., vol. 141, pp. 13-18, which is incorporated herein by reference), or related model term-selection techniques can instead be used to find a set of genes that distinguish well between the classes, as described in the U.S. provisional application “Use of fast orthogonal search and other model-building techniques for interpretation of gene expression profiles”, filed Nov. 3, 2000. This is described next.

Use of Fast Orthogonal Search and Other Model-Building Techniques for Interpretation of Gene Expression Profiles

Here we show how model-building techniques, such as fast orthogonal search (FOS) and the orthogonal search method (OSM), can be used to analyze gene expression profiles and predict the class to which a profile belongs.

A gene expression profile pⱼ can be thought of as a column vector containing the expression levels e_{i,j}, i=1, . . . ,I of l genes. We suppose that we have J of these profiles for training, so that j=1, . . . ,J. Each of the profiles pⱼ was created from a sample, e.g., from a tumor, belonging to some class. The samples may be taken from patients diagnosed with various classes of leukemia, e.g., acute lymphoblastic leukemia (ALL) or acute myeloid leukemia (AML), as in the paper by Golub et al. (1999).

Given a training set of profiles belonging to known classes, e.g., ALL and AML, the problem is to create a predictor that will assign a new profile to its correct class. The method described here has some similarity to that by Golub et al. (1999). However, the use of FOS, OSM, or an analogous form of model building is not disclosed in that paper. Indeed, the class predictor created here through the use of OSM is different and correctly classified more profiles in an independent set, using less training data, than in Golub et al. (1999).

First, consider distinguishing between two classes. To distinguish between ALL and AML classes, Golub et al. (1999) defined “an ‘ideal expression pattern’ corresponding to a gene that was uniformly high [1] in one class and uniformly low [0] in the other”. They then selected a set of “informative genes” that were “chosen based on their correlation with the class distinction”, and used these genes in a voting scheme to classify a given expression profile. In particular, the “set of informative genes to be used in the predictor was chosen to be the 50 genes most closely correlated with ALL-AML distinction in the known samples”. A possible drawback of this approach is that some genes that might always have near-duplicate expression levels could be selected, which may unfairly bias the voting.

Similar to how “ideal expression pattern” was defined (Golub et al., 1999), we define the output y(i) of an ideal classifier to be −1 for each profile pⱼ from the first class, and 1 for each profile pⱼ from the second class. For each of the i genes, i=1, . . . ,l, define

gᵢ(j)=e_{i,j}, (1)

the expression level of the i-th gene in the j-th training profile, j=1, . . . ,J. We then wish to choose a subset \tilde{g}ₘ(j), m=1, . . . ,M, of the/candidate functions gᵢ(j) to approximate y(j) by

$$y ( j ) = {\sum }_{m = 1}^{M} {a}_{m} {\tilde{g}}_{m} ( j ) + r ( j ) \qquad ( 2 )$$

where aₘ is the coefficient for each term in the series, and where r(j) is the model error, so as to minimize the mean-square error (MSE)

$$MSE = \frac{1}{J} {\sum }_{j = 1}^{j} {( r ( j ) )}^{2} \qquad ( 3 )$$

The subset \tilde{g}ₘ(j) m=1, . . . ,M, containing the model terms can be found by using FOS or OSM to search efficiently through the larger set of candidate functions gᵢ(j), i=₁, . . . ,l, as follows. In succession, we try each of the/candidate functions and measure the reduction in MSE if that candidate alone were best-fit, in the mean-square sense, to y(i), i.e., if M=1 in Eq. (2). The candidate for which the MSE reduction would be greatest is chosen as the first term for the model in Eq. (2). To find the second term for the model, we set M=2. Then each of the remaining 1-1 candidates is orthogonalized relative to the chosen model term. This enables the MSE reduction to be efficiently calculated were any particular candidate added as the second term in the model. We select the candidate for which the MSE reduction would be greatest to be the second model term, and so on.

In this scheme, candidate functions are orthogonalized with respect to already-selected model terms. After the orthogonalization, a candidate whose mean-square would be less than some threshold value is barred from selection (Korenberg 1989 a, b). This prevents numerical errors associated with fitting orthogonalized functions having small norms. It prevents choosing near duplicate candidate functions, corresponding to genes that always have virtually identical expression levels.

In fact, to increase efficiency, the orthogonal functions need not be explicitly created. Rather, FOS uses a Cholesky decomposition to rapidly assess the benefit of adding any candidate as a further term in the model. The method is related to, but more efficient than, a technique proposed by Desrochers (1981), “On an improved model reduction technique for nonlinear systems”, Automatica, Vol. 17, pp. 407-409. The selection of model terms can be terminated once a pre-set number have been chosen. For example, since each candidate function gᵢ(j) is defined only for J values of j, there can be at most J linearly independent candidates, so that at most J model terms can be selected. (However, there will typically be far more than J candidates that are searched.) In addition, a stopping criterion, based on a standard correlation test (Korenberg 1989b), can be employed. Alternatively, various tests such as the Information Criterion, described in Akaike (1974) “A new look at the statistical model identification”, IEEE Trans. Automatic Control, Vol. 19, pp. 716-723, or an F-test, discussed e.g. in Soderstrom (1977) “On model structure testing in system identification”, Int. J. Control, Vol. 26, pp. 1-18, can be used to stop the process.

Once the model terms have been selected for Eq. (2), the coefficients am can be immediately obtained from quantities already calculated in carrying out the FOS algorithm. Further details about OSM and FOS are contained in the cited papers. The FOS selection of model terms can also be carried out iteratively (Adeney and Korenberg, 1994) for possibly increased accuracy.

Once the model of Eq. (2) has been determined, it can then function as a predictor as follows. If P_{J+1} is a novel expression profile to be classified, then let \tilde{g}ₘ(J+1) be the expression level of the gene is this profile corresponding to the m-th model term in Eq. (2). (This gene is typically not the m-th gene in the profile, since \tilde{g}ₘ(j), the m-th model term, is typically not gₘ(j), the m-th candidate function.) Then evaluate

$$z = {\sum }_{m = 1}^{M} {a}_{m} {\tilde{g}}_{m} ( J + 1 ) , \qquad ( 4 )$$

and use a test of similarity to compare z with −1 (for the first class) and 1 (for the second class). For example, if z<0, the profile may be predicted to belong to the first class, and otherwise to the second class.

Alternatively, suppose that MSE₁ and MSE₂ are the MSE values for the training profiles in classes 1 and 2 respectively. For example, the calculation to obtain MSE, is carried out analogously to Eq. (3), but with the averaging only over profiles in class 1. The MSE₂ is calculated similarly for class 2 profiles. Then, assign the novel profile p_{J+1} to class 1 if

$$\frac{{( z + 1 )}^{2}}{{MSE}_{1}} < \frac{{( z - 1 )}^{2}}{{MSE}_{2}} , \qquad ( 5 )$$

and otherwise to class 2. In place of using a mean-square test of similarity, analogous tests using absolute values or a power higher than 2 can be employed.

Alternatively, once the model terms for Eq. (2) have been selected by FOS, the genes to which they correspond can then be used as a set of “informative genes” in a voting scheme such as described by Golub et al. (1999).

Above, for simplicity, we have used the expression level of one gene to define a candidate function, as in Eq. (1). However, we can also define candidate functions in terms of powers of the gene's expression level, or in terms of crossproducts of two or more genes expression levels, or the candidate functions can be other functions of some of the genes' expression levels. Also, in place of the raw expression levels, the logarithm of the expression levels can be used, after first increasing any negative raw value to some positive threshold value (Golub et al., 1999).

While FOS avoids the explicit creation of orthogonal functions, which saves computing time and memory storage, other procedures can be used instead to select the model terms and still conform to the invention. For example, an orthogonal search method (Desrochers, 1981; Korenberg, 1989 a, b), which does explicitly create orthogonal functions can be employed, and one way of doing so is shown in Example 4 below. Alternatively, a process that does not involve orthogonalization can be used. For example, the set of candidate functions is first searched to select the candidate providing the best fit to y(j), in a mean-square sense, absolute value of error sense, or according to some other criterion of fit. Then, for this choice of first model term, the remaining candidates are searched to find the best to have as the second model term, and so on. Once all model terms have been selected, the model can be “refined” by reselecting each model term, each time holding fixed all other model terms (Adeney and Korenberg, 1994).

In an alternative embodiment, one or more profiles from each of the two classes to be distinguished can be spliced together to form a training input. The corresponding training output can be defined to be −1 over each profile from the first class, and 1 over each profile from the second class. The nonlinear system having this input and output could clearly function as a classifier, and at least be able to distinguish between the training profiles from the two classes. Then FOS can be used to build a model that will approximate the input output behavior of the nonlinear system (Korenberg 1989 a, b) and thus function as a class predictor for novel profiles.

It will also be appreciated that the class distinction to be made may be based on phenotype, for example, the clinical outcome in response to treatment. In this case, the invention described herein can be used to establish genotype phenotype correlations, and to predict phenotype based on genotype.

Finally, while distinctions between two classes have been considered above, predictors for more than two classes can be built analogously. For example, the output y(j) of the ideal classifier can be defined to have a different value for profiles from different classes. Alternatively, as is well known, the multi-class predictor can readily be realized by various arrangements of two-class predictors.

### EXAMPLE 4

The first 11 ALL profiles (#1-#11 of Golub et al. first data set), and all 11 of the AML profiles (#28-#38 of the same data set), formed the training data. These 22 profiles were used to build 10 concise models of the form in Eq. (2), which were then employed to classify profiles in an independent set in Golub et al. (1999). The first 7000 gene expression levels in each profile were divided into 10 consecutive sets of 700 values. For example, to build the first model, the expression levels of genes 1-700 in each training profile were used to create 700 candidate functions gᵢ(j). These candidates were defined as in Eq. (1), except that in place of each raw expression level e_{i,j}, its log was used:

gᵢ(j)=log₁₀e_{i,j}, i=1, . . . ,700 j=1, . . . 22,

after increasing to 100 any raw expression value that was less. Similarly, genes 701-1400 of each training profile were used to create a second set of 700 candidate functions, for building a second model of the form in Eq. (2), and so on. The training profiles had been ordered so that the pⱼ, for j=1, . . . ,11 corresponded to the ALL profiles, and for j=12, . . . ,22 to the AML profiles. Hence the training output was defined as

$$\begin{matrix}y ( j ) = - 1 , j = 1 , … , 11 \\ = 1 , j = 12 , … , 22\end{matrix}$$

The following procedure was used to find each model. First, each of the 700 candidate functions was tried as the only model term in Eq. (2) (with M=1), and its coefficient chosen to minimize the MSE given by Eq. (3). The candidate for which the MSE was smallest was selected as the first model term \tilde{g}₁(j). For j=1, . . . ,22, define a first orthogonal function as \tilde{w}₁(j)=\tilde{g}₁(j), with its coefficient c₁. Assume that the model already has M terms, for M≧1, and a (M+1)-th term is sought. For j=1, . . . ,22, let \tilde{w}ₘ(j) be orthogonal functions created from the model chosen terms \tilde{g}ₘ(j), m=1, . . . ,M. Let \tilde{c}ₘ be the corresponding coefficients of the \tilde{w}ₘ(j), where these coefficients were found to minimize the mean-square error of approximating y(j) by a linear combination of the \tilde{w}ₘ(j), m=1, . . . ,M. Then, for each candidate gᵢ(j) not already chosen for the model, the modified Gram-Schmidt procedure is used to create a function orthogonal to the \tilde{w}ₘ(j), m=1, . . . ,M. Thus, for j=1, . . . ,22, set

$${w}_{M + 1}^{( 1 )} ( j ) = {g}_{i} ( j ) - {α}_{M + 1 , 1} {\tilde{w}}_{1} ( j ) , \\ where$$

$${α}_{M + 1 , 1} = \frac{{\sum }_{j = 1}^{22} {g}_{i} ( j ) {\tilde{w}}_{1} ( j )}{{\sum }_{j = 1}^{22} {( {\tilde{w}}_{1} ( j ) )}^{2}} .$$

And, for r=2, . . . ,M, and j=1, . . . ,22, set

$${w}_{M + 1}^{( r )} ( j ) = {w}_{M + 1}^{( r - 1 )} ( j ) - {α}_{M + 1 , r} {\tilde{w}}_{r} ( j )$$

$$where$$

$${α}_{M + 1 , r} = \frac{{\sum }_{j = 1}^{22} {w}_{M + 1}^{( r - 1 )} ( j ) {\tilde{w}}_{r} ( j )}{{\sum }_{j = 1}^{22} {( {\tilde{w}}_{r} ( j ) )}^{2}}$$

The function w_{M+1}^{(M)}(j) (which was created from the candidate gᵢ(j)) is orthogonal to the \tilde{w}ₘ(j), m=1, . . . ,M. If the candidate gi(j) were added to the model as the (M+1)-th term, then it follows readily that the model MSE would equivalently be

$$e = \frac{1}{22} {\sum }_{j = 1}^{22} {( y ( j ) - {\sum }_{m = 1}^{M} {\tilde{c}}_{m} {\tilde{w}}_{m} ( j ) - {c}_{M + 1} {w}_{M + 1}^{( M )} ( j ) )}^{2}$$

$$where$$

$${c}_{M + 1} = \frac{{\sum }_{j = 1}^{22} y ( j ) {w}_{M + 1}^{( M )} ( j )}{{\sum }_{j = 1}^{22} {( {w}_{M + 1}^{( M )} ( j ) )}^{2}}$$

Therefore, the candidate gᵢ(j) for which e is smallest is taken as the (M+1)-th model term \tilde{g}_{M+1}(j), the corresponding

$${w}_{M + 1}^{( M )} ( j )$$

becomes \tilde{w}_{M+1}(j), and the corresponding c_{M+1} becomes \tilde{c}_{M+1}. Once all model terms \tilde{g}ₘ(j) have been selected, their coefficients aₘ that minimize the MSE can be readily calculated (Korenberg, 1989a,b).

Each of the 10 models was limited to five model terms. The terms for the first model corresponded to genes #697, #312, #73, #238, #275 and the model % MSE (expressed relative to the variance of the training output) was 6.63%. The corresponding values for each of the 10 models are given in Table 1. The coefficients aₘ of the model terms \tilde{g}ₘ(j) were obtained for each model by least-squares fitting to the training output y(j), j=1, . . . ,22.

TABLE 1

| Mod- | Gene # | Gene # | Gene # | Gene # | Gene # | % |
|---|---|---|---|---|---|---|
| el # | (1^{st} Term) | (2^{nd} Term) | (3^{rd} Term) | (4^{th} Term) | (5^{th} Term) | MSE |
| 1 | 697 | 312 | 73 | 238 | 275 | 6.63 |
| 2 | 760 | 1350 | 1128 | 741 | 935 | 3.63 |
| 3 | 1779 | 1909 | 1745 | 2025 | 1505 | 4.72 |
| 4 | 2288 | 2354 | 2267 | 2385 | 2389 | 3.53 |
| 5 | 3252 | 3413 | 2822 | 3348 | 3434 | 3.98 |
| 6 | 4107 | 4167 | 4095 | 3507 | 3694 | 1.85 |
| 7 | 4847 | 4368 | 4539 | 4211 | 4565 | 2.02 |
| 8 | 5191 | 4951 | 4946 | 5502 | 5132 | 3.51 |
| 9 | 6200 | 6094 | 5950 | 5626 | 6158 | 2.82 |
| 10 | 6696 | 6308 | 6347 | 6727 | 6857 | 4.65 |

The 10 identified models were then used to classify profiles in an independent, second data set from Golub et al. (1999), which contained 20 ALL and 14 AML test profiles. For each model, the value of z was calculated using Eq. (4) with M=5, and with \tilde{g}ₘ(J+1) equal to the log of the expression level of the gene in the test profile corresponding to the m-th model term. For example, for model #1, \tilde{g}₁(J+1), \tilde{g}₂(J+1), . . . ,\tilde{g}₅(J+1) corresponded to the log of the expression level of gene #697, #312, . . . ,#275 respectively, in the test profile. The values of z for the 10 models were summed; if the result was negative, the test profile was classified as ALL, and otherwise as AML.

By this means, all 20 of the test ALL, and all 14 of the test AML, profiles in the independent set were correctly classified. These classification results, after training with 22 profiles, compare favorably with those for the Golub et al. method. Golub et al. (1999) used the entire first data set of 38 profiles to train their ALL-AML predictor, which then was able to classify correctly all of the independent set except for five where the prediction strength was too low for a decision.

In order to investigate how small an individual model could be and still allow the combination to be an effective classifier, the procedure was repeated using the above sets of 700 candidate functions, but this time to build 10 two-term models, which are summarized in Table 2.

TABLE 2

| Model # | Gene # (1^{st} Term) | Gene # (2^{nd} Term) | % MSE |
|---|---|---|---|
| 1 | 697 | 312 | 23.08 |
| 2 | 760 | 1350 | 22.01 |
| 3 | 1779 | 1909 | 25.52 |
| 4 | 2288 | 2354 | 26.73 |
| 5 | 3252 | 3413 | 18.09 |
| 6 | 4107 | 4167 | 16.27 |
| 7 | 4847 | 4368 | 12.62 |
| 8 | 5191 | 4951 | 19.38 |
| 9 | 6200 | 6094 | 23.08 |
| 10 | 6696 | 6308 | 40.42 |

Again, a test profile was classified by calculating the value of z for each model using Eq. (4), this time with M=2, and then adding the values of z for the 10 models; if the result was negative, the test profile was classified as ALL, and otherwise as AML. By this means, all 20 of the test ALL, and 13 of the 14 test AML, profiles in the independent set were correctly classified.

Moreover, it was found that considerably less than full training profiles sufficed to maintain this level of accuracy for the two-term models. The identical classification accuracy was obtained with only models #1-#6 (whose construction required only the first 4200 genes of each training profile), or with additional models. The first 4200 gene expression levels in each of 22 profiles that sufficed for training here represent less than 40% of the data used by Golub et al. (1999).

It should be noted that individually the models made a number of classification errors, ranging from 1-17 errors for the two-term and from 2-11 for the five-term models. This was not unexpected since each model was created after searching through a relatively small subset of 700 expression values to create the model terms. However, the combination of several models resulted in excellent classification.

The principle of this aspect of the present invention is to separate the values of the training gene expression profiles into subsets, to find a model for each subset, and then to use the models together for the final prediction, e.g. by summing the individual model outputs or by voting. Moreover, the subsets need not be created consecutively, as above. Other strategies for creating the subsets could be used, for example by selecting every 10^{th} expression level for a subset.

This principle can increase classification accuracy over that from finding a single model using entire gene expression profiles. Note that here, since the output y(j) was defined only for j=1, . . . ,22, at most 22 independent terms could be included in the model (which would allow no redundancy), but identifying a number of models corresponding to the different subsets allows the contributions of many more genes to be taken into account. Indeed, searching through the first 7000 expression levels to find a five-term model, using the same 22 training profiles, resulted in a %MSE of 1.33%, with terms corresponding to genes #6200, 4363, 4599, 4554, and 3697. However, this model was not particularly accurate, misclassifying 4 of the 20 ALL, and 5 of the 14 AML, profiles in the independent set.

Finally, it will be appreciated that the principle of dividing the training profiles into subsets, and finding models for the subsets, then using the models in concert to make the final classification decisions, is not confined to use of FOS, OSM, or any particular model-building technique. For example, a parallel cascade model can be found for each subset as described earlier above, and then the models can be used together to make the final predictions.

Prediction of Treatment Response Using Gene Expression Profiles

This section concerns prediction of clinical outcome from gene expression profiles using work in a different area, nonlinear system identification. In particular, the approach can predict long-term treatment response from data of a landmark article by Golub et al. (1999), which to the applicant's knowledge has not previously been achieved with these data. The present paper shows that gene expression profiles taken at time of diagnosis of acute myeloid leukemia contain information predictive of ultimate response to chemotherapy. This was not evident in previous work; indeed the Golub et al. article did not find a set of genes strongly correlated with clinical outcome. However, the present approach can accurately predict outcome class of gene expression profiles even when the genes do not have large differences in expression levels between the classes.

Prediction of future clinical outcome, such as treatment response, may be a turning point in improving cancer treatment. This has previously been attempted via a statistically-based technique (Golub et al., 1999) for class prediction based on gene expression monitoring, which showed high accuracy in distinguishing acute lymphoblastic leukemia (ALL) from acute myeloid leukemia (AML). The technique involved selecting “informative genes” strongly correlated with the class distinction to be made, e.g., ALL versus AML, and found families of genes highly correlated with the latter distinction (Golub et al., 1999). Each new tissue sample was classified based on a vote total from the informative genes, provided that a “prediction strength” measure exceeded a predetermined threshold. However, the technique did not find a set of genes strongly correlated with response to chemotherapy, and class predictors of clinical outcome were less successful.

In a sample of 15 adult AML patients treated with anthracycline-cytarabine, eight failed to achieve remission while seven achieved remissions of 46-84 months. Golub et al. “found no evidence of a strong multigene expression signature correlated with clinical outcome, although this could reflect the relatively small sample size”. While no prediction results for clinical outcome were presented in the paper, they stated that such class predictors were “not highly accurate in cross-validation”. Similarly, Schuster et al. (2000, “Tumor Identification by Gene Expression Profiles: A Comparison of Five Different Clustering Methods”, Critical Assessment of Microarray Data Analysis CAMDA'00) could not predict therapy response using the same data in a study of five different clustering techniques: Kohonen-clustering, Fuzzy-Kohonen-network, Growing cell structures, K-means-clustering, and Fuzzy-K-means-clustering. They found that none of the techniques clustered the patients having similar treatment response. The ALL-AML dataset from Golub et al. (1999) was one of two specified for participants in the CAMDA'00 meeting, and to the applicant's knowledge none reported accurate prediction of treatment response with these data.

Prediction of survival or drug response using gene expression profiles can be achieved with microarrays specialized for non-Hodgkin's lymphoma (Alizadeh et al., 2000, “Distinct types of diffuse large B-cell lymphoma identified by gene expression profiling”, Nature Vol. 403, 503-511) involving some 18,000 cDNAs, or via cluster analysis of 60 cancer cell lines and correlation of drug sensitivity of the cell lines with their expression profiles (Scherf, U., Ross, D. T., Waltham, M., Smith, L. H., Lee, J. K. & Tanabe, L. et al., 2000, “A gene expression database for the molecular pharmacology of cancer”, Nature Genet. Vol. 24, 236-244). Also, using clustering, Alon et al. (1999, “Broad patterns of gene expression revealed by clustering analysis of tumor and normal colon tissues probed by oligonucleotide arrays”, Proc. Natl. Acad. Sci. USA Vol. 96, 6745-6750) showed that tumor and normal classes could be distinguished even when the genes used had small average differences between the classes.

In the present section, it is shown that a technique for modeling nonlinear systems, called parallel cascade identification (PCI) (Korenberg, 1991) can accurately predict class from gene expression profiles even when the genes do not have large differences in expression levels between the classes. In particular, the technique is able to predict long-term treatment response to chemotherapy with anthracycline-cytarabine, which to the applicant's knowledge was not previously possible with the data from Golub et al. The work in this section shows that gene expression profiles taken at time of diagnosis, and lacking a set of genes strongly correlated with clinical outcome, still enable prediction of treatment response otherwise only evident several years later.

Although the sample size of the AML treatment response group is not large, there are several reasons why the class prediction method used here, and its performance over this group, is significant for interpreting gene expression profiles. First, since to the applicant's knowledge neither the Golub et al. (1999) method nor any other published to date has accurately predicted treatment response for this group of patients, it may serve as a benchmark for gauging the sensitivity of new methods. Second, the successful predictions of treatment response by the method used here are readily shown to be statistically significant on well-known tests that examined two different aspects of the prediction. Third, the same method was also shown above to perform well on the ALL vs AML task. While the latter class distinction is much easier, the resulting performance indicates that the proposed method for class prediction has more general applicability for interpreting gene expression profiles. Certainly, because of the simplicity of the ALL/AML distinction, minor differences in the number of profiles correctly classified would not necessarily indicate how different methods would compare on more difficult problems such as prediction of treatment response. However, the performance observed here is also consistent with that in classifying protein sequences (Korenberg et al., 2000a) where the method has been successfully tested on thousands of novel sequences (Korenberg et al., 2000b).

To develop a means for predicting clinical outcome from gene expression profiles, begin by viewing the problem as one of nonlinear system identification, using a “black box” approach. Here, the system to be identified is defined by one or more inputs and one or more outputs; the problem is to build a model whose input/output relation approximates that of the system, with no a priori knowledge of the system's structure. Construct a training input by splicing together the expression levels of genes from profiles known to correspond to failed and to successful treatment outcomes. Define the training output as −1 over input segments corresponding to failed outcomes, and 1 over segments corresponding to successful outcomes. The nonlinear system having this input/output relation would clearly function as a classifier, at least for the profiles used in forming the training input. A model is then identified to approximate the defined input/output behavior, and can subsequently be used to predict the class of new expression profiles. Below, only the first failed and first successful outcome profiles were used to construct the training input; the remaining seven failed and six successful outcome profiles served as tests. The same data were used as in Golub et al. (1999). All samples had been obtained at time of leukemia diagnosis. Each profile contained the expression levels of 6817 human genes (Golub et al., 1999), but because of duplicates and additional probes in the Affymetrix microarray, in total 7129 gene expression levels were present in the profile.

Nonlinear system identification has already been used for protein family prediction (Korenberg et al., 2000 a,b), and a useful feature of PCI (Korenberg, 1991) is that effective classifiers may be created using very few training data. For example, one exemplar from each of the globin, calcium-binding, and kinase families sufficed to build parallel cascade two-way classifiers that outperformed (Korenberg et al., 2000b), on over 16,000 test sequences, state-of-the-art hidden Markov models trained with the same exemplars. The parallel cascade method and its use in protein sequence classification are reviewed in Korenberg et al. (2001).

While input x(i) to a parallel cascade model will represent the expression levels of genes, both input and output of the model will be treated as if they were time-series data. The rationality of considering the gene expression values as a time series is now justified, in view of the fact that genes in a profile are not ordered sequentially. In fact, lack of actual time dependency causes no problem: PCI simply looks for a pattern in the data. This point is illustrated by the type of training data that could be used to identify the protein sequence classifiers, e.g. FIG. 2(a) of Korenberg et al. (2000b). There, five-digit binary codes were employed to represent each amino acid in a protein sequence, resulting in subtly different plaid-like patterns for different protein families. Though these patterns were artificial in that they depended upon the five-digit codes used, parallel cascade models could be trained to distinguish between the patterns and thus classify novel protein sequences.

For the approach to work, it is necessary that (1) training exemplars from different classes produce different patterns, and (2) the memory length of the nonlinear system to be identified is of appropriate size to “capture” the pattern for each class. Analogously, to learn how to distinguish between two wood grains, say mahogany and cherry, given one table of each, a much smaller sampling window than an entire tabletop would suffice to make a decision. Moreover, sliding the sampling window over both tabletops would provide multiple training examples of each grain.

### EXAMPLE 5

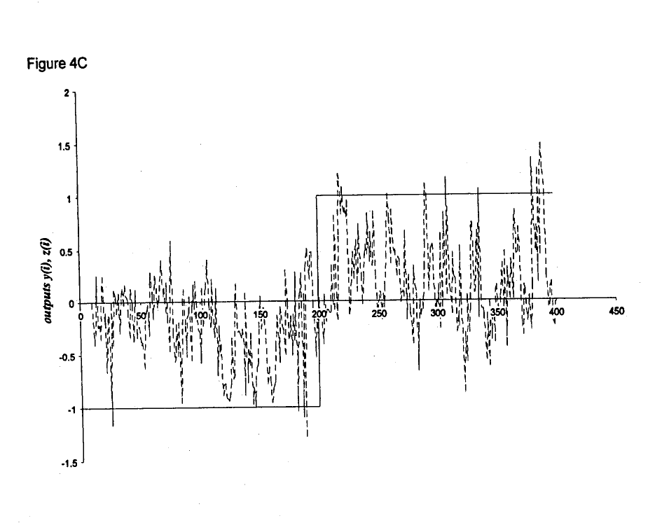

The set of failed outcomes was represented by profiles #28-#33, #50, #51 of data from Golub et al. (1999), and the set of successful outcomes by profiles #34-#38, #52, #53. Raw expression levels of 200 selected genes from the first “failed treatment” profile #28 and first “successful treatment” profile #34 were concatenated to form training input x(i) (FIG. 4A). The genes selected were the 200 having greatest difference in expression levels between the two profiles. Order of appending the selected genes resulted in an almost white input (FIG. 4B), which is typically advantageous for nonlinear system identification, including PCI. (The selected genes had the same relative ordering as in the original profiles, and this ordering caused the input autocovariance to be closest to a delta function, out of several orderings tried.) Corresponding training output y(i) was defined as −1 over the “failed treatment” segment of the input and 1 over the “successful treatment” segment (solid line, FIG. 4C). For this input and output, a model was identified using PCI (Korenberg, 1991). The identified model clearly functions as a “predictor” of treatment response, at least for expression profiles #28 and #34. Indeed, when training input x(i) is fed through the parallel cascade model, resulting output z(i) is predominately negative (average value: −0.238) over the “failed treatment” segment, and predominately positive (average value: 0.238) over the “successful treatment” segment of the input (dashed line, FIG. 4C). The identified model had a mean-square error (MSE) of about 74.8%, expressed relative to the variance of the output signal.

Care was taken to ensure that the test sequences were treated independently from the training data. First, the two profiles used to form the training input were never used as test profiles. Second, the set used to determine a few parameters chiefly relating to model architecture never included the profile on which the resulting model was tested. Thus a model was never trained, nor selected as the best of competing models, using data that included the test profile.

In order to identify the parallel cascade model, four parameters relating mostly to its structure had to be pre-specified. These were: (i) the memory length of the dynamic linear element L that began each cascade, (ii) the degree of the polynomial static nonlinearity N that followed, (iii) the maximum number of cascades allowed in the model, and (iv) a threshold concerning the minimum reduction in MSE required before a candidate cascade could be accepted into the model (Korenberg, 1991). How these parameter settings were determined is explained next.

As noted, only two profiles were used to construct the training input, which left 13 profiles for testing. Each time, 12 of these 13 were used to determine values for the above parameters, and then the parallel cascade model having the chosen parameter settings was tested on the remaining (“held out”) profile This process was repeated until each of the 13 profiles had been tested. The 12 profiles used to determine the parameter values will be referred to as the evaluation set, which never included the profile held out for testing.

The parameter values were determined each time by finding the choice of memory length, polynomial degree, maximum number of cascades allowed, and threshold that resulted in fewest errors in classifying the 12 profiles. The limit on the number of cascades allowed actually depended on the values of the memory length and polynomial degree in a trial. The limit was set to ensure that the number of variables introduced into the model was significantly less than the number of output points used in the identification. Effective combinations of parameter values did not occur sporadically. Rather, there were ranges of the parameters, e.g. of memory length and threshold values, for which the corresponding models were effective classifiers. When the fewest errors could be achieved by more than one combination of parameter values, then the combination was selected that introduced fewest variables into the model. If there was still more than one such combination, then the combination of values where each was nearest the middle of the effective range for the parameter was chosen. An upper limit of 15 cascades was allowed in the model to ensure that there would be significantly fewer variables introduced than output points used in the identification.

There was in fact one tie, for two different threshold values, in best performance over the 12-profile evaluation set when profile #53 was held out for testing. The fewest errors resulted, with fewest variables introduced into the model (7 cascades), when the memory length was 12, the polynomial degree was 7, and the threshold T was either 11 or 12. However, equally good classification over the evaluation set was observed, though with more model variables, for T=10 (13 cascades), whereas the classification degraded considerably for T=13. Hence the threshold T=11 was chosen since a threshold of 12 was closer to the margin for good performance.

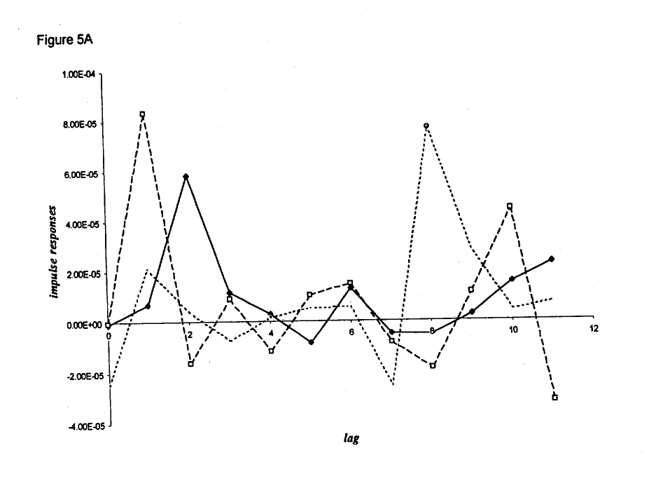

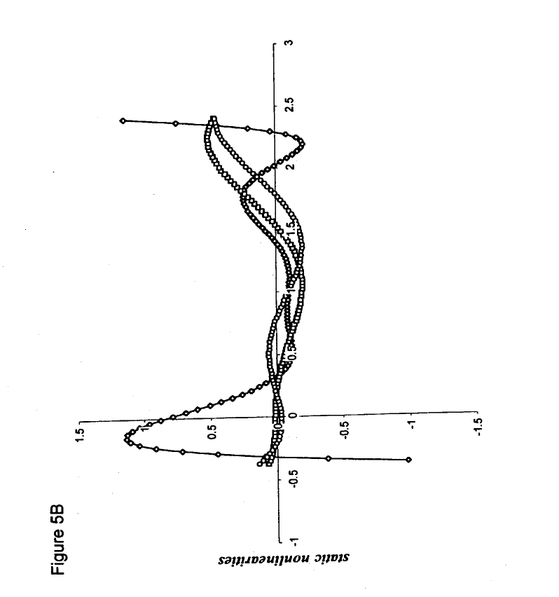

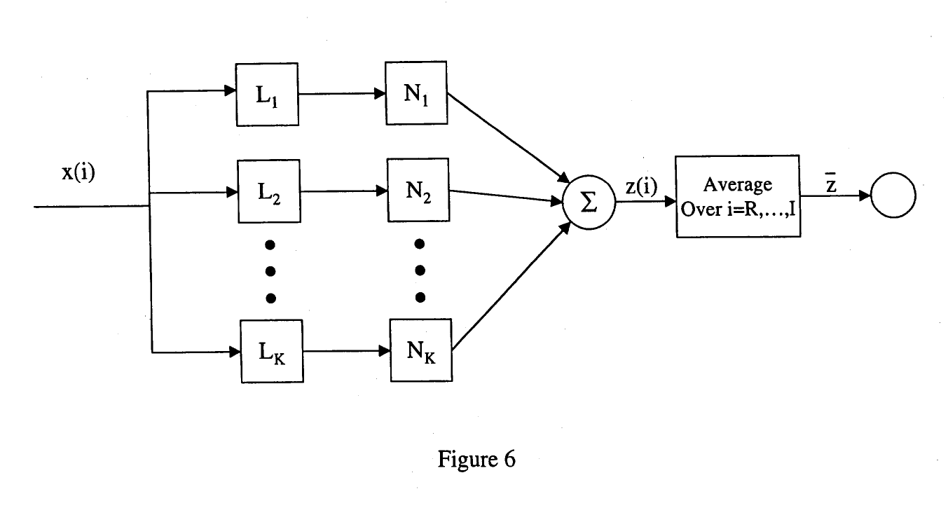

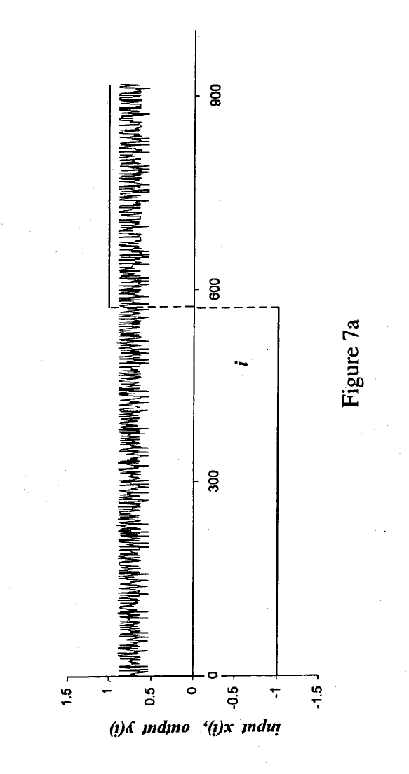

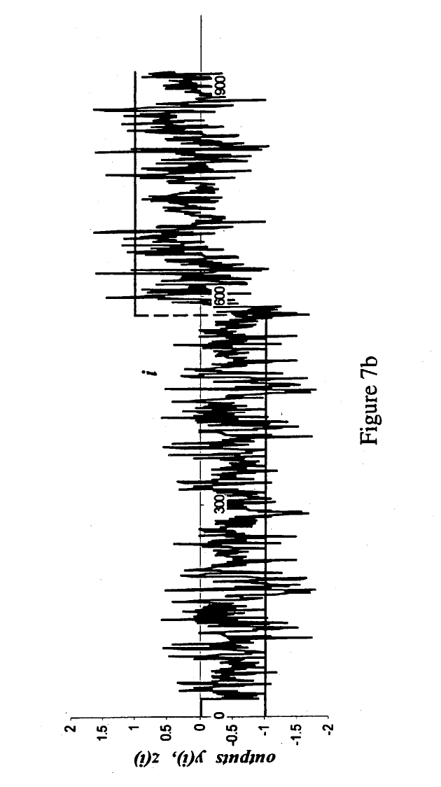

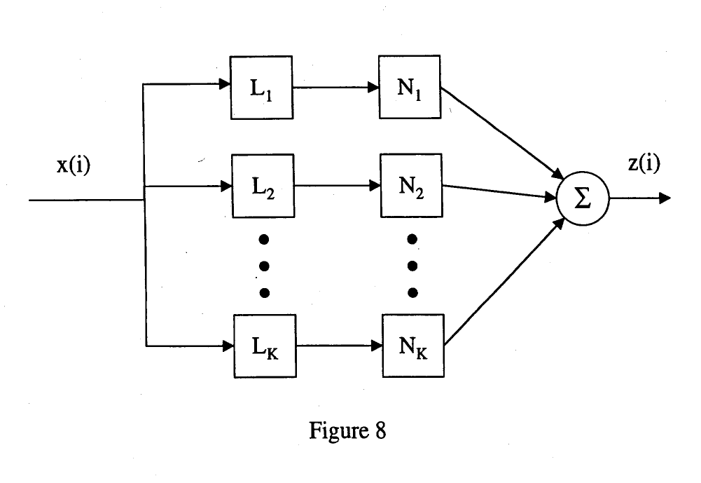

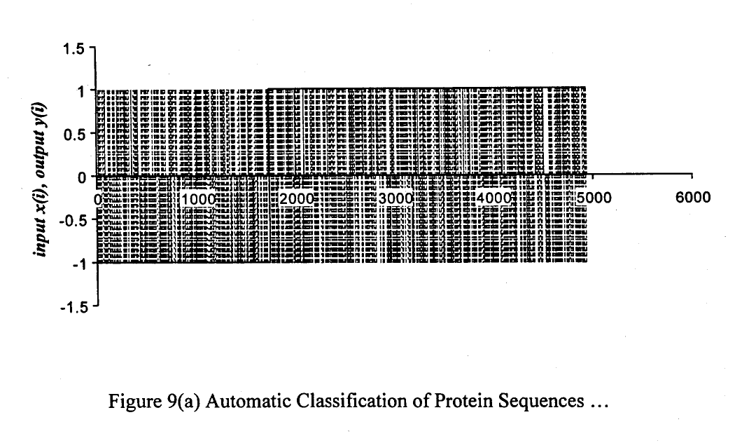

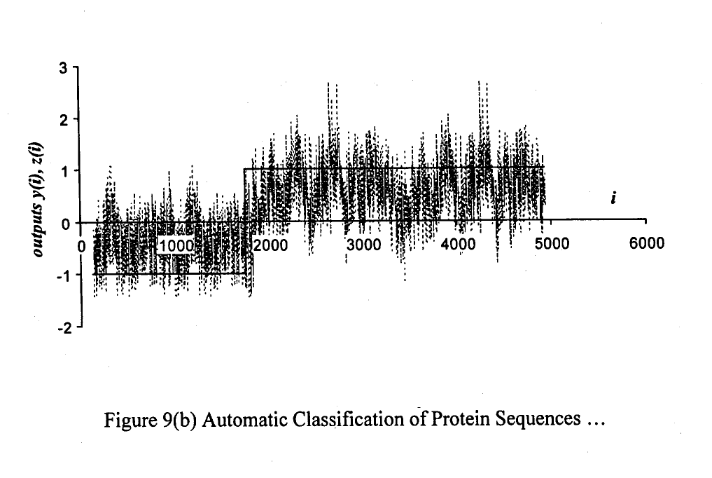

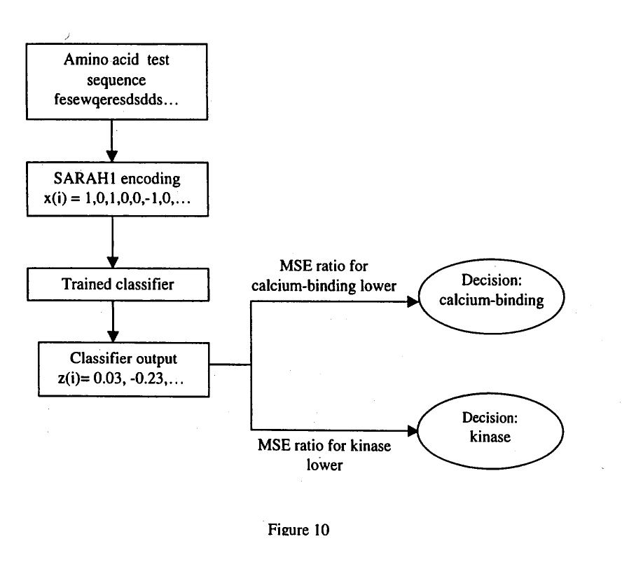

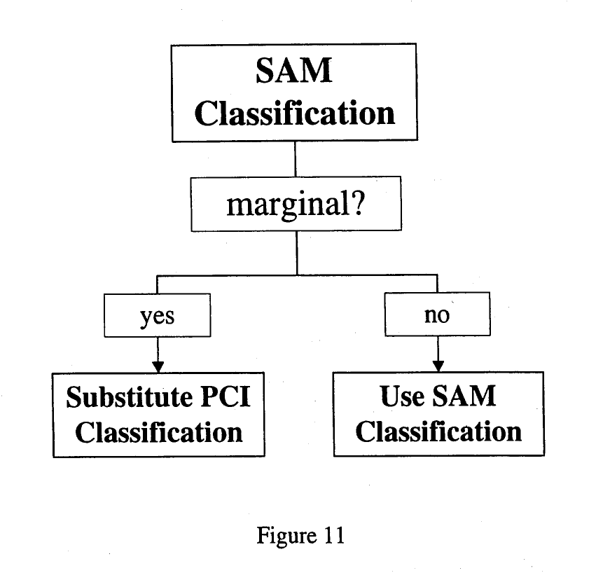

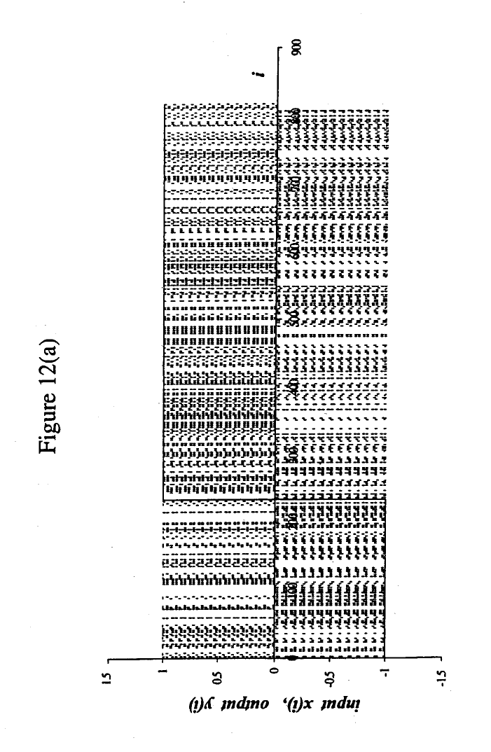

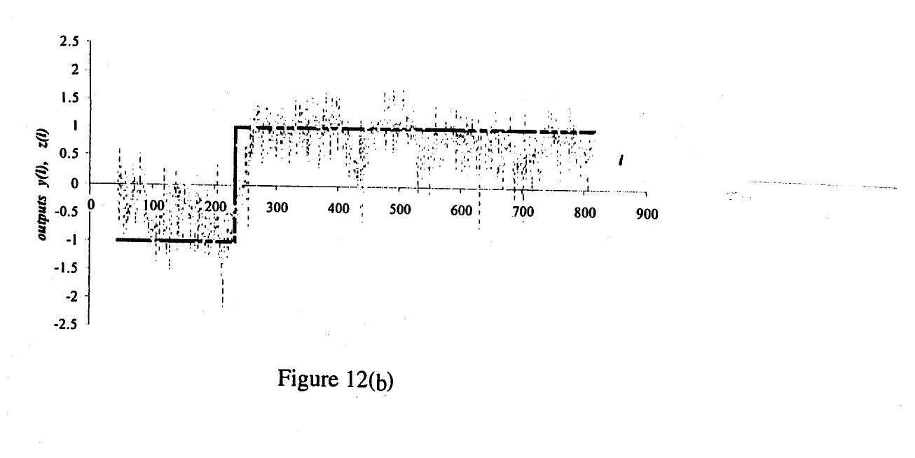

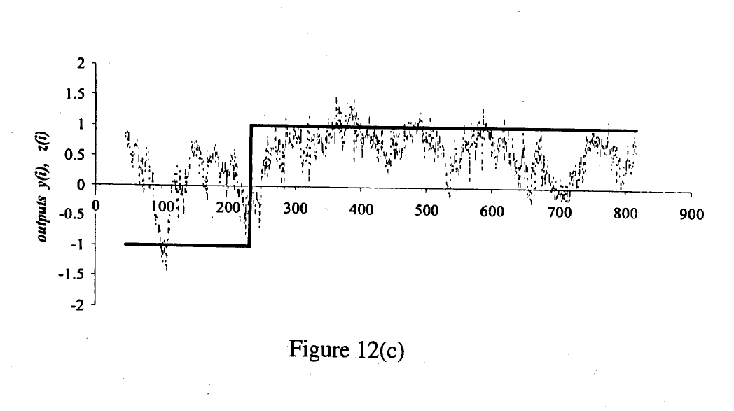

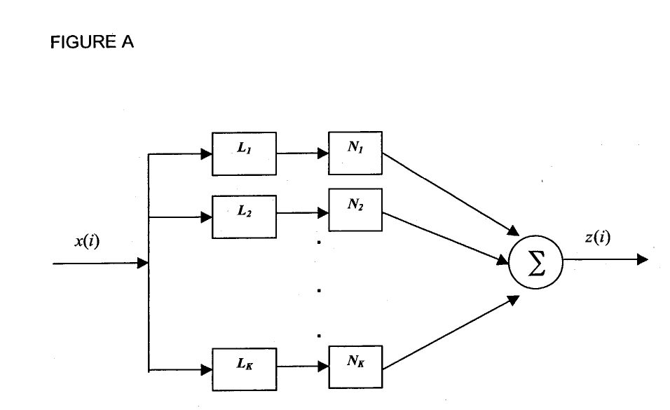

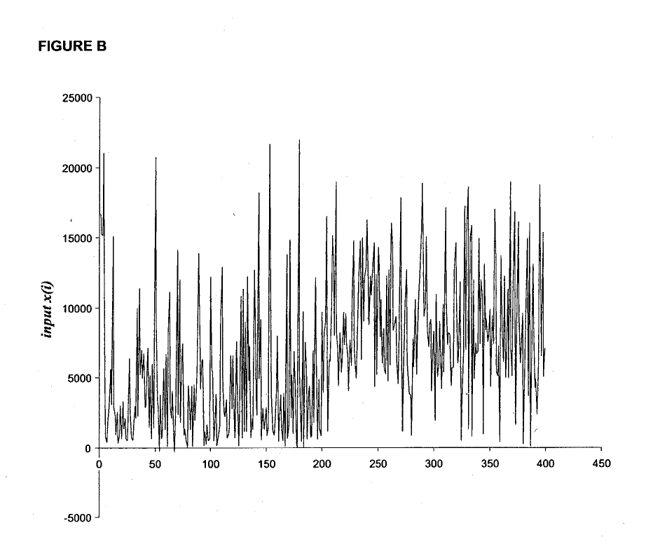

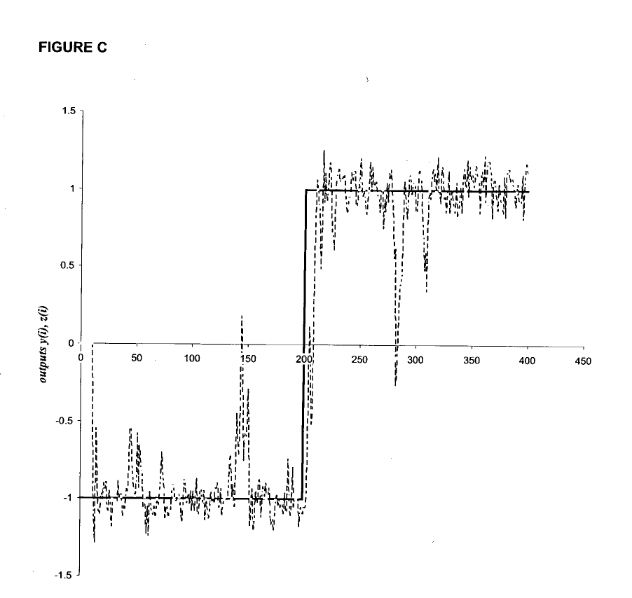

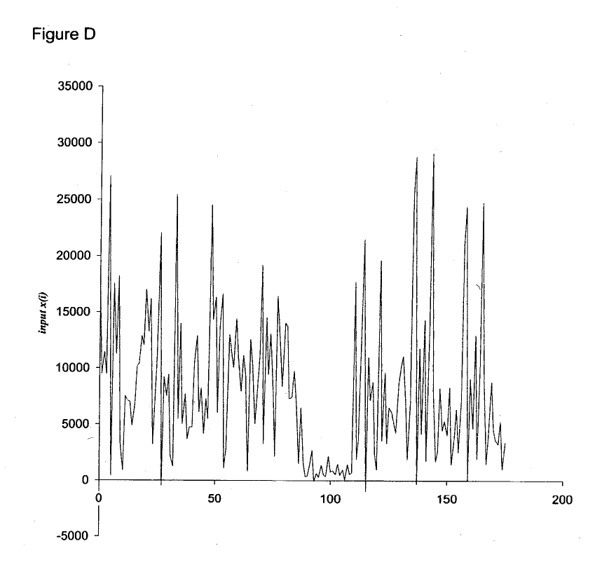

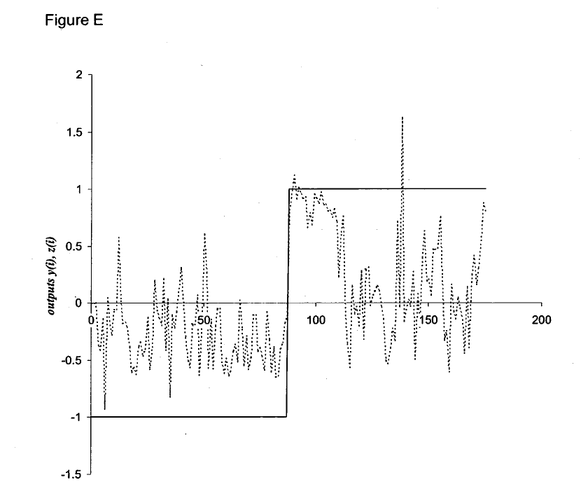

Each time it was found that it was possible to achieve good performance (2-3 errors depending on the 12 profiles in the evaluation set), and employ few cascade variables, if the same four parameter values were used. This meant that the identical parallel cascade model was in fact chosen for each classification of the held out profile. This model had 7 cascades in total, each beginning with a linear element having memory length of 12, followed by a 7^{th} degree polynomial static nonlinearity. FIG. 5A shows the impulse response functions of the linear elements in the 2^{nd}, 4^{th}, and 6^{th} cascades, and 5B the corresponding polynomial static nonlinearities that followed.

Each time, the profile held out for testing was classified by appending, in the same order as used above, the raw expression levels of genes in the profile to form an input signal. This input was then fed through the identified model, and its mean output was used to classify the profile. If the mean output was negative, the profile was classified as “failed treatment”, and if positive as “successful treatment”. This decision criterion was taken from the earlier protein classification study (Korenberg et al., 2000a).

Results

The parallel cascade model correctly classified 5 of the 7 “failed treatment” (F) profiles and 5 of the 6 “successful treatment” (S) profiles. The corresponding Matthews' correlation coefficient (Matthews, 1975, “Comparison of the predicted and observed secondary structure of T4 phage lysozyme”, Biochim. Biophys. Ac., Vol. 405, 442-451) was 0.5476. Two different aspects of the parallel cascade prediction of treatment response were tested, and both times reached statistical significance. First, the relative ordering of profiles from the two outcome types by their model mean outputs was tested by the Mann-Whitney test, a non-parametric test to determine whether the model detected differences between the two profile types. The second aspect of the PCI prediction concerned how well the individual values of the classifier output for the 7 F and 6 S test profiles correlated with the class distinction.

How did the model mean outputs order the test profiles from the two outcome types? If the parallel cascade model did not detect differences between the two types, then the relative ordering of F and S profiles by value of mean output should be random. The parallel cascade mean outputs were ranked as shown in Table 1, which indeed demonstrates that mean outputs for F profiles tend to be less than those for S profiles. The corresponding Mann-Whitney test statistics were U=8, U=34. This meant that the way the parallel cascade model distinguished between F and S profiles was significant at the 0.0367 level on a one-tailed test, for n₁=7, n₂=6. A one-tailed test was used because, due to the way the model had been trained, the mean output was expected to be less for a failed than for a successful outcome profile.

Next, the identified parallel cascade model was found to act as a transformation that converts input sequences of 200 gene expression values each into output signals whose individual values correlate with the F vs S class distinction. Because the model had a memory length of 12, the first 11 points of each output signal were excluded to allow the model to “settle”, so that the 13 test profiles gave rise to 13×189=2457 output values. Of these, 1323 output values corresponded to the 7 F, and 1134 to the 6 S, test profiles. For the S profiles, the proportion of output values that were positive was 109% of the corresponding proportion for F profiles, while the S profiles' proportion of output values that were negative was 90% that for F profiles. Indeed, with a Yates-corrected chi-square of 5.13 (P<0.0235), output values corresponding to test S profiles tended to be positive more often, and negative less often, than those corresponding to test F profiles. Finally, the actual values of the outputs also correlated with the F vs S distinction. The 1323 output values corresponding to the test F profiles totaled −535.93, while the 1134 output values for test S profiles totaled 3209.14. Indeed, point biserial correlation showed that there was a highly significant relation between the actual values of the output signals for the test profiles and the F vs S class distinction (P<0.0102). Recall that the model's memory length was 12. Hence, limiting the point biserial correlation to every 12^{th} output value would avoid any overlap in gene expression levels used to obtain such output values. The relation of these 208 output values to the F vs S distinction is still highly significant (P<0.0155).

PCI is only one approach to predicting treatment response and other methods can certainly be applied. Importantly, it has been shown here to be possible to predict long-term response of AML patients to chemotherapy using the Golub et al. data, and now wider implications can be considered. For example, the method for predicting clinical outcome described here may have broader use in cancer treatment and patient care. In particular, it has recently been shown that there are significant differences in the gene expression profiles of tumors with BRCA1 mutations, tumors with BRCA2 mutations, and sporadic tumors (Hedenfalk et al, 2001, “Gene-expression profiles in hereditary breast cancer”, N. Engl. J. Med., Vol. 344, 539-548). The present method may be used to distinguish the gene expression profiles of these tumor classes, predict recurrence, and assist in selection of treatment regimen.

TABLE 3
Parallel cascade ranking of test expression profiles

| Rank | Mean Output | Actual Outcome | Profile # |
|---|---|---|---|
| 1 | −1.17 | F | 31 |
| 2 | −0.863 | F | 32 |
| 3 | −0.757 | F | 33 |
| 4 | −0.408 | S | 37 |
| 5 | −0.298 | F | 50 |
| 6 | −0.0046 | F | 30 |
| 7 | 0.0273 | S | 53 |
| 8 | 0.078 | S | 38 |
| 9 | 0.110 | F | 51 |
| 10 | 0.148 | F | 29 |
| 11 | 0.194 | S | 52 |
| 12 | 0.267 | S | 36 |
| 13 | 16.82 | S | 35 |

F = failed treatment, S = successful treatment.
The complete set of profiles is found in http://www-enome.wi.mit.edu/MPR/data_set_ALL_AML.html (see Golub et al, 1999) and “Profile #” follows the same numbering scheme.

Examples 1-3 and 5 can be briefly summarized by the following steps:

1. Compare the gene expression levels in the training profiles and select a set of genes that assist in distinguishing between the classes. Each profile contained 7129 expression values (from 6817 human genes plus duplicates and additional probes) but only 200 values were selected from each profile. Consider first the 2-class distinction, with one training profile available from each class. For each selected gene, the absolute value of the difference between the corresponding raw values on the two training profiles ranked in the top 200 of all the genes. When there were more training profiles from each class, say 15 exemplars from the acute lymphoblastic leukemia (ALL) class and 11 from the acute myeloid leukemia (AML) class, then the first 4 AML training profiles were repeated to givel5 AML training profiles. For each gene, the mean of its raw expression levels was computed over the 15 ALL training profiles, and the mean was also computed over the 15 AML training profiles. Then the absolute value of the difference between the two means was computed for the gene. The 200 genes having the largest of such absolute values were selected. If instead the model is to distinguish. n-classes, n>2, a criterion could be based on a sum of absolute values of pairwise differences between the means of a gene's expression levels, where each mean is computed over the training profiles for a class.

2. Append values representative of the expression levels of the selected genes from a given profile to produce a segment representative of the class of that profile. Repeat for each profile, maintaining the same order of appending the values representative of the expression levels. For predicting treatment response in Example 5, the raw expression levels as given in the data downloaded from the MIT website

http://www-genome.wi.mit.edu/MPR/data set ALL AML.html

were the values appended. For the ALL-AML distinction task, in Example 3 where multiple training profiles were used from each class, logarithms (to base 10) of the raw expression levels were used, after increasing to 100 any smaller raw level. The order used to append the values is preferably chosen to assist in distinguishing between the classes. Not all orders are equally good. It was observed that an appending order that causes the training input autocovariance to be nearly a delta function tends to be beneficial. One method for finding such an appending order is through appropriate use of a stochastic minimization technique involving trial interchanges within a segment, while maintaining the same order in all segments. This technique is readily obtained by adapting the method described in Hunter and Kearney, 1983, “Generation of random sequences with jointly specified probability density and autocorrelation functions”, Biol. Cybern., Vol. 47, pp. 141-146. A preferred way to choose an appending order is by checking the resulting model's classification accuracy over an evaluation set of known profiles from the classes to be distinguished. In fact, in Examples 1-3 and 5, the selected values were always appended in the same relative order that they appeared in the full profile.

3. Concatenate the representative segments to form a training input.

4. Define an input/output relation by creating a training output having values corresponding to the input values, where the output has a different value over each representative segment from a different class.

5. Identify a parallel cascade model (FIG. 1) to approximate the input/output relation.

Classify a new gene expression profile by (a) appending the expression levels of the same genes selected above, in the same order as above, to produce a segment for input into the identified parallel cascade model; (b) applying the segment to the parallel cascade model and obtaining the corresponding output; and (c) using the output to make a prediction of the class of the new expression profile. One decision criterion, for the two-class case is: if the mean of the parallel cascade output is less than zero, then assign the profile to the first class, and otherwise to the second class. Another criterion (used in Example 3) is based on certain ratios of mean square error (MSE). This criterion compares the MSE of the model output z(i) from −1, relative to the corresponding MSE over the ALL training segments, with the MSE of z(i) from 1, relative to the MSE over the AML training segments.

In the above, models have been built to distinguish between two or more classes of interest. However, it will be appreciated that separate models could instead be built for each class using PCI, FOS, OSM, or other model-building techniques. One way to do so is, for each class, to use at least one profile exemplar to obtain a training input comprising a sequence of values. Next, for each class, obtain a training output by shifting the input signal to advance the sequence. Then, for each class, find a finite-dimensional system to approximate the relation between the training input and output for that class.

Once the models have been built for each class, a query profile (i.e., a profile whose class is to be determined) can be classified in one of two ways. First, an input signal and an output signal can be made from the query profile, then the input is fed through each of the models for the classes, and the model outputs are compared with the output derived from the query profile. The closest “fit” determines the class, using a criterion of similarity such as minimum Euclidean distance. Second, the input and output signals derived from the query profile can be used to find a model, which is then compared with the class models, and the closest one determines the classification of the query profile.

It will be appreciated that the class predictors described herein can be combined with other predictors, such as that of Golub et al. (1999), nearest neighbor classifiers, classification trees, and diagonal linear discriminant analysis.

The above material has described use of nonlinear system identification techniques such as parallel cascade identification, fast orthogonal search, the orthogonal search method, and other methods of model building to interpret gene expression profiles. However, it will be appreciated that the present invention also applies to many other diagnostic profiles representative of biological information, such as proteomics data. Proteomics techniques, for example the use of two-dimensional electrophoresis (2-DE), enables the analysis of hundreds or thousands of proteins in complex mixtures such as whole-cell lysates. (See http://www.uku.fi/˜lehesran/proteomics6.htm) Proteome analysis is an effective means of studying protein expression, and has an advantage over use of gene expression profiles, since mRNA levels do not correlate very highly with protein levels. Protein separation through use of 2-DE gels occurs as follows. In the first dimension, proteins are separated by their iso-electric points in a pH gradient. In the second dimension, proteins are separated according to their molecular weights. The resulting 2-DE image can be analyzed, and quantitative values obtained for individual spots in the image. Protein profiles may show differences due to different conditions such as disease states, and comparing profiles can detect proteins that are differently expressed. Such proteomics data can also be interpreted using the present invention, e.g. for diagnosis of disease or prediction of clinical outcome.

(B) Protein Sequence Classification and Recognition of Active Sites, Such as Phosphorylation and ATP-Binding Sites, on Proteins

A recent paper (Korenberg et al., 2000a) introduced the approach of using nonlinear system identification as a means for automatically classifying protein sequences into their structure/function families. The particular technique utilized, known as parallel cascade identification (PCI), could train classifiers on a very limited set of exemplars from the protein families to be distinguished and still achieve impressively good two-way classifications. For the nonlinear system classifiers to have numerical inputs, each amino acid in the protein was mapped into a corresponding hydrophobicity value, and the resulting hydrophobicity profile was used in place of the primary amino acid sequence. While the ensuing classification accuracy was gratifying, the use of (Rose scale) hydrophobicity values had some disadvantages. These included representing multiple amino acids by the same value, weighting some amino acids more heavily than others, and covering a narrow numerical range, resulting in a poor input for system identification. Another paper (Korenberg et al., 2000b) introduced binary and multilevel sequence codes to represent amino acids, for use in protein classification. The new binary and multilevel sequences, which are still able to encode information such as hydrophobicity, polarity, and charge, avoid the above disadvantages and increase classification accuracy. Indeed, over a much larger test set than in the original study, parallel cascade models using numerical profiles constructed with the new codes achieved slightly higher two-way classification rates than did hidden Markov models (HMM) using the primary amino acid sequences, and combining PCI and HMM approaches increased accuracy.

There are at least two areas where the PCI method can be usefully employed in protein sequence classification. First, it may be an aid to individual scientists engaged in various aspects of protein research. This is because the method can create effective classifiers after training on very few exemplars from the families to be distinguished, particularly when binary (two-way) decisions are required. This can be an advantage, for instance, to researchers who have newly discovered an active site on a protein, have only a few examples of it, and wish to accelerate their search for more by screening novel sequences. Second, as discussed below, the classifiers produced by the approach have the potential of being usefully employed with hidden Markov models to enhance classification accuracy.

In order to have some comparison of performance when both HMM and PCI approaches receive either the primary amino acid sequences or equivalent information, a new scheme for coding the amino acids was used in Korenberg et al. (2000b). The scheme equally weights each amino acid, and causes greater variability in the numerical profiles representing the protein sequences, resulting in better inputs for system identification. At the same time, the relative ranking imposed by the Rose scale can be almost completely preserved. The scheme is similar to, but more elaborate than, one used in recent work on distinguishing exon from intron DNA sequences (M. J. Korenberg, E. D. Lipson, and J. E. Solomon, “Parallel Cascade Recognition of Exon and Intron DNA Sequences”, appended hereto as Appendix C).

In the latter problem, it was necessary to find a numerical representation for the bases adenine (A), thymine (T), guanine (G), and cytosine (C). In work concerned with efficiently comparing two DNA sequences via crosscorrelation (Cheever et al., 1989, “Using Signal Processing Techniques for DNA Sequence Comparison”, in Proc. Northeastern Bioengineeing Symposium, Boston, Mass., pp. 173-174) it was suggested that the bases A, T, G, C be represented by respectively 1, −1, i, −i, where i=\sqrt{−1}. Directly using these complex numbers to represent the bases in DNA sequences would require two inputs (for the real and imaginary parts). In order to avoid the need for two-input parallel cascade models, this representation was modified, so that bases A, T, G, C in a sequence were encoded respectively by ordered pairs (0, 1), (0, −1), (1, 0), (−1, 0). This doubled the length of the sequence, but allowed use of a single real input. Note that here purines A, G are represented by pairs of same sign, as are pyrimidines C, T. Provided that this biochemical criterion was met, good classification would result. Also, many other binary representations were explored, such as those using only +1 as entries, but it was found that within a given pair, the entries should not change sign. For example, representing a base by (1, −1) did not result in a good classifier.

The same approach was applied in Korenberg et al. (2000b) to develop numerical codes for representing each of the amino acids. Thus, within the code for an amino acid, the entries should not change sign. To represent the 20 amino acids, each of the codes could have 5 entries, three of them 0, and the other two both 1 or both −1. There are (₂⁵)=10 such codes of each sign, so the 20 amino acids can be uniquely coded this way. Next, the codes are preferably not randomly assigned to the amino acids, but rather in a manner that adheres to a relevant biochemical property. Consequently, the amino acids were ranked according to the Rose hydrophobicity scale (breaking ties), and then the codes were assigned in descending value according to the binary numbers corresponding to the codes.

The resulting scale in Table 1, where the bottom half is the negative mirror image of the top half, will be referred to as SARAH1 (“Simultaneously Axially and Radially Aligned Hydrophobicities”). In a similar scale, SARAH2 in Table 2, each code again has 5 entries, but here two of them are 0, while the other three all equal 1 or all equal −1.

TABLE 2
SARAH2 Scale

| Amino Acid | Binary Code |
|---|---|
| C | 1,1,1,0,0 |
| F | 1,1,0,1,0 |
| I | 1,1,0,0,1 |
| V | 1,0,1,1,0 |
| L | 1,0,1,0,1 |
| W | 1,0,0,1,1 |
| M | 0,1,1,1,0 |
| H | 0,1,1,0,1 |
| Y | 0,1,0,1,1 |
| A | 0,0,1,1,1 |
| G | 0,0,−1,−,−1 |
| T | 0,−1,0,−1,−1 |
| S | 0,−1,−1,0,−1 |
| R | 0,−1,−1,−1,0 |
| P | −1,0,0,−1,−1 |
| N | −1,0,−1,0,−1 |
| D | −1,0,−1,−1,0 |
| Q | −1,−1,0,0,−1 |
| E | −1,−1,0,−1,0 |
| K | −1,−1,−1,0,0 |

TABLE 1
SARAH1 SCALE

| Amino Acid | Binary Code |
|---|---|
| C | 1,1,0,0,0 |
| F | 1,0,1,0,0 |
| I | 1,0,0,1,0 |
| V | 1,0,0,0,1 |
| L | 0,1,1,0,0 |
| W | 0,1,0,1,0 |
| M | 0,1,0,0,1 |
| H | 0,0,1,1,0 |
| Y | 0,0,1,0,1 |
| A | 0,0,0,1,1 |
| G | 0,0,0,−1,−1 |
| T | 0,0,−1,0,−1 |
| S | 0,0,−1,−1,0 |
| R | 0,−1,0,0,−1 |
| P | 0,−1,0,−1,0 |
| N | 0,−1,−1,0,0 |
| D | −1,0,0,0,−1 |
| Q | −1,0,0,−1,0 |
| E | −1,0,−1,0,0 |
| K | −1,−1,0,0,0 |

Using one of these scales to encode a protein sequence yields a numerical profile 5 times longer than the original sequence, but one where each amino acid is uniquely represented and equally weighted. Alternatively, either of the scales can be employed to translate a protein sequence into a profile consisting of 5 signals, which leads to use of 5-input parallel cascade models (Korenberg, 1991). The greater range covered by the new numerical profiles, compared with the (Rose scale) hydrophobicity profiles, results in improved inputs for training the parallel cascade models, and more accurate classification as shown below.

While the above scales carry information about hydrophobicity, scales can similarly be constructed to imbed other chemical or physical properties of the amino acids such as polarity, charge, alpha-helical preference, and residue volume. Since each time the same binary codes are assigned to the amino acids, but in an order dependent upon their ranking by a particular property, the relative significance of various factors in the protein folding process can be studied in this way. It is clear that randomly assigning the binary codes to the amino acids does not result in effective parallel cascade classifiers. In addition, the codes can be concatenated to carry information about a number of properties. In this case, the composite code for an amino acid can have 1, −1, and 0 entries, and so can be a multilevel rather than binary representation.

The application of nonlinear system identification to automatic classification of protein sequences was introduced in Korenberg et al. (2000a). Briefly, begin by choosing representative sequences from two or more of the families to be distinguished, and represent each sequence by a profile corresponding to a property such as hydrophobicity or to amino acid sequence. Then splice these profiles together to form a training input, and define the corresponding training output to have a different value over each family or set of families that the classifier is intended to recognize.

For example, consider building a binary classifier intended to distinguish between calcium-binding and kinase families using their numerical profiles constructed according to the SARAH1 scale. The system to be constructed is shown in FIG. 1, and comprises a parallel array of cascades of dynamic linear and static nonlinear elements. We start by splicing together the profiles for the calcium-binding sequence 1SCP and kinase sequence 1PFK, to form a 4940 point training input x(i). The input has this length because the 1SCP and 1PFK sequences have 348 and 640 amino acids respectively and, as the SARAH1 scale is used in this example, each amino acid is replaced with a code 5 digits long. However, as noted above, the scale could have instead been used to create 5 signals, each 988 points in length, for a 5-input parallel cascade model. No preprocessing of the data is employed. Define the corresponding training output y(i) to be −1 over the calcium-binding, and 1 over the kinase, portions of the input. We now seek a dynamic (i.e., has memory) nonlinear system which, when stimulated by the training input, will produce the training output. Clearly, such a system would function as a binary classifier, and at least would be able to distinguish apart the calcium-binding and the kinase representatives.

We can build an approximation to such a dynamic system, given the training input and output, using techniques from nonlinear system identification. The particular technique we have used in this paper, called parallel cascade identification, is more fully explained in Korenberg (1991), and is summarized below.

Suppose that x(i), y(i), i=0, . . . ,I, are the training input and output (in this example, I=4939). Then parallel cascade identification is a technique for approximating the dynamic nonlinear system having input x(i) and output y(i) by a sum of cascades of alternating dynamic linear (L) and static nonlinear (N) elements.

The parallel cascade identification method (Korenberg, 1991) can be outlined as follows. A first cascade of dynamic linear and static nonlinear elements is found to approximate the dynamic nonlinear system. The residual, i.e., the difference between the system and the cascade outputs, is calculated, and treated as the output of a new dynamic nonlinear system. A cascade of dynamic linear and static nonlinear elements is now found to approximate the new system, the new residual is computed, and so on. These cascades are found in such a way as to drive the crosscorrelations of the input with the residual to zero. It can be shown that any dynamic nonlinear discrete-time system having a Volterra or a Wiener functional expansion can be approximated, to an arbitrary degree of accuracy in the mean-square sense, by a sum of a sufficient number of the cascades (Korenberg, 1991). For generality of approximation, it suffices if each cascade comprises a dynamic linear element L followed by a static nonlinearity N, and this LN structure was used in the present work, and is assumed in the algorithm description given immediately below.

In more detail, suppose that yₖ(i) denotes the residual after the k-th cascade has been added to the model, with y₀(i)=y(i). Let zₖ(i) be the output of the k-th cascade. Then, for k=1, 2, . . . ,

yₖ(i)=yₖ₋₁(i)−zₖ(i). (1)

Assume that the output y(i) depends on input values x(i),x(i−1), . . . , x(i−R), i.e., upon input lags 0, . . . ,R. There are many ways to determine a suitable (discrete) impulse response function hₖ(j), j=0, . . . ,R for the linear element L beginning the k-th cascade (Korenberg, 1991). One practical solution is to set it equal to either the first order crosscorrelation of the input x(i) with the current residual Yₖ₋₁(i), or to a slice of a higher order input/residual crosscorrelation, with discrete impulses added or subtracted at diagonal values. Thus, set

hₖ(j)=φ_{xy}_{ₖ₋₁}(j) (2)

if the first order input residual crosscorrelation φ_{xy}_{ₖ₋₁} is used, or

hₖ(j)=φ_{xxy}_{ₖ₋₁}(j,A)±Cδ(j−A) (3)

if the second order crosscorrelation φ_{xxy}_{ₖ₋₁} is used, and similarly if a higher order crosscorrelation is instead employed. In Eq. (3), the discrete impulse function δ(j−A)=1, j=A, and equals 0 otherwise. If Eq. (3) is used, then A is fixed at one of 0, . . . ,R, the sign of the δ-term is chosen at random, and C is adjusted to tend to zero as the mean-square of the residual yₖ₋₁ approaches zero. If the third order input residual crosscorrelation φ_{xxxy}_{ₖ₋₁} were used, then we would have analogously

hₖ(j)=φ_{xxy}_{ₖ₋₁}(j,A₁,A₂)±C₁δ(j−A₁)±C₂δ(j−A₂) (4)

where A₁, A₂, C₁, C₂ are defined similarly to A, C in Eq. (3).

However, it will be appreciated that many other means can be used to determine the hₖ(j), and that the approach is not limited to use of slices of crosscorrelations. More details of the parallel cascade approach are given in Korenberg (1991). Once the impulse response hₖ(j) has been determined, the linear element's output uₖ(i) can be calculated as

$${u}_{k} ( i ) = {\sum }_{j = 0}^{R} {h}_{k} ( j ) x ( i - j ) . \qquad ( 5 )$$

In Eq. (5), the input lags needed to obtain the linear element's output range from 0 to R, so its memory length is R+1.

The signal uₖ(i) is itself the input to a static nonlinearity in the cascade, which may be represented by a polynomial. Since each of the parallel cascades in the present work comprised a dynamic linear element L followed by a static nonlinearity N, the latter's output is the cascade output

$${z}_{k} ( i ) = {\sum }_{d = 0}^{D} {a}_{k d} {u}_{k}^{d} ( i ) . \qquad ( 6 )$$

The coefficients a_{kd} defining the polynomial static nonlinearity N may be found by best-fitting, in the least-square sense, the output zₖ(i) to the current residual yₖ₋₁(i). Once the k-th cascade has been determined, the new residual yₖ(i) can be obtained from Eq. (1), and because the coefficients a_{kd} were obtained by best-fitting, the mean-square of the new residual is

$$\overline{{y}_{k}^{2} ( i )} = \overline{{y}_{k - 1}^{2} ( i )} - \overline{{z}_{k}^{2} ( i )} , \qquad ( 7 )$$

where the overbar denotes the mean (time average) operation. Thus, adding a further cascade to the model reduces the mean-square of the residual by an amount equal to the mean-square of the cascade output (Korenberg, 1991). This, of course, by itself does not guarantee that the mean-square error (MSE) of approximation can be made arbitrarily small by adding sufficient cascades. However, the cascades can be created in such a way as to drive the input/residual crosscorrelations arbitrarily close to zero for a sufficient number of cascades. This is the central point that guarantees convergence to the least-square solution, for a given memory length R+1 of the dynamic linear element and polynomial degree D of the static nonlinearity. It implies that in the noise-free case a discrete-time system having a Volterra or a Wiener functional expansion can be approximated arbitrarily accurately by a finite sum of these LN cascades. For a proof of convergence, see Korenberg (1991).

Once the parallel cascade model has been identified, we calculate its output due to the training input, and also the MSE of this output from the training output for calcium-binding and kinase portions of the training input. Recall that the training output has value −1 over the calcium-binding portion, and 1 over the kinase portion, of the training input. Hence we compute a first MSE of the model output from −1 for the calcium-binding portion, and a second MSE from 1 for the kinase portion, of the training input.

The parallel cascade model can now function as a binary classifier via an MSE ratio test. A sequence to be classified, in the form of its numerical profile x(i) constructed according to the SARAH1 scale, is fed to the model, and we calculate the corresponding output

$$z ( i ) = {\sum }_{k = 1}^{K} {z}_{k} ( i ) , \qquad ( 8 )$$

where K is the number of cascade paths in the final model. Next, we compare the MSE of z(i) from −1, relative to the corresponding MSE for the appropriate training sequence, with the MSE of z(i) from 1, again relative to the MSE for the appropriate training sequence. In more detail, the first ratio is

$${r}_{1} = \frac{\overline{{( z ( i ) + 1 )}^{2}}}{{e}_{1}} , \qquad ( 9 )$$

where e₁ is the MSE of the parallel cascade output from −1 for the training numerical profile corresponding to calcium-binding sequence 1SCP. The second ratio computed is

$${r}_{2} = \frac{\overline{{( z ( i ) - 1 )}^{2}}}{{e}_{2}} , \qquad ( 10 )$$

where e₂ is the MSE of the parallel cascade output from 1 corresponding to kinase sequence 1PFK. Each time an MSE is computed, we commence the averaging on the (R+1)-th point. If r₁ is less than r₂, the sequence is classified as calcium-binding, and if greater, as kinase. This MSE ratio test has also been found to be an effective classification criterion in distinguishing exon from intron DNA sequences (Korenberg, Lipson, Solomon, unpublished). As noted below, an effective memory length R+1 for our binary classifiers was 125, corresponding to a primary amino acid sequence length of 25, which was therefore the minimum length of the sequences which could be classified by the models identified in the present example.

Other decision criteria may be used. For example, distributions of output values corresponding to each training input may be computed. Then, to classify a novel sequence, compute the distribution of output values corresponding to that sequence, and choose the training distribution from which it has the highest probability of coming. However, only the MSE ratio criterion just discussed was used to obtain the results in the present example.

Note that, instead of splicing together only one representative sequence from each family to be distinguished, several representatives from each family can be joined (Korenberg et al., 2000a). It is preferable, when carrying out the identification, to exclude from computation those output points corresponding to the first R points of each segment joined to form the training input. This is done to avoid introducing error into the identification due to the transition zones where the different segments of the training input are spliced together.

By means of this parallel cascade identification and the appropriate training input and output created as described earlier, three classifier models were found, each intended to distinguish between one pair of families. For example, a parallel cascade model was identified to approximate the input/output relation defined to distinguish calcium-binding from kinase sequences. The three models corresponded to the same assumed values for certain parameters, namely the memory length R+1, the polynomial degree D, the maximum number of cascades permitted in the model, and a threshold for deciding whether a cascade's reduction of the MSE justified its inclusion in the model. To be acceptable, a cascade's reduction of the MSE, divided by the mean-square of the current residual, had to exceed the threshold T divided by the number of output points I₁ used to estimate the cascade, or equivalently:

$$\overline{{z}_{k}^{2} ( i )} > \frac{T}{{I}_{1}} \overline{{y}_{k - 1}^{2} ( i )} \qquad ( 11 )$$

This criterion (Korenberg, 1991) for selecting candidate cascades was derived from a standard correlation test.

The parallel cascade models were identified using the training data for training calcium-binding vs kinase classifiers, or on corresponding data for training globin vs calcium-binding or globin vs kinase classifiers. Each time the same assumed parameter values were used, the particular combination of which was analogous to that used in the DNA study. In the latter work, it was found that an effective parallel cascade model for distinguishing exons from introns could be identified when the memory length was 50, the degree of each polynomial was 4, and the threshold was 50, with 9 cascades in the final model. Since in the DNA study the bases are represented by ordered pairs, whereas here the amino acids are coded by 5-tuples, the analogous memory length in the present application is 125. Also, the shortest of the three training inputs here was 4600 points long, compared with 818 points for the DNA study. Due to the scaling factor of 5/2 reflecting the code length change, a roughly analogous limit here is 20 cascades in the final models for the protein sequence classifiers. In summary, the default parameter values used in the present example were memory length (R+1) of 125, polynomial degree D of 4, threshold T of 50, and a limit of 20 cascades. As shown in FIG. 2b of Korenberg (2000b), when the training input of FIG. 2a of that paper is fed through the calcium-binding vs kinase classifier, the resulting output is indeed predominately negative over the calcium-binding portion, and positive over the kinase portion, of the input. The next section concerns how the identified parallel cascade models performed over the test sets.

Classification Results for Test Sequences

All results presented here are for two-way classification, based on training with a single exemplar from the globin, calcium-binding, and kinase families.

Original Test Set Used in Korenberg et al. (2000a)

The detailed results are shown in Table 3 for the SARAH1 and SARAH2 encoding schemes, with correct classifications by PCI averaging 85% and 86% respectively. These should be compared with a 79% average success rate in the earlier study (Korenberg et al., 2000a) where Rose scale hydrophobicity values were instead used to represent the amino acids.

TABLE 3
Correct Classification Rate for PCI on Original Test Set

| | | | | |
|---|---|---|---|---|
| | % Correct | % Correct | % Correct | |
| Encoding | Globin v Cal- | Globin v | Cal-Bind | Mean % |
| Scale | Bind | Kinase | v Kinase | Correct |

| | | | | | | | |
|---|---|---|---|---|---|---|---|
| SARAH1 | 84 | 100 | 73 | 100 | 83 | 67 | 85% |
| SARAH2 | 85 | 100 | 79 | 100 | 85 | 67 | 86% |

Large Test Set

The detailed results are shown in Table 4 for PCI using the SARAH1 encoding scheme, for the hidden Markov modeling approach using the Sequence Alignment and Modeling System (SAM) (http://www.cse.ucsc.edu/research/compbio/sam.html), and for the combination of SAM with PCI. The SARAH2 scheme was not tested on this set. The combination was implemented as follows. When the HMM probabilities for the two families to be distinguished were very close to each other, the SAM classification was considered to be marginal, and the PCI result was substituted. The cutoff used with SAM was 1 in the NLL-NULL score, which is the negative log of the probability of a match. This cutoff was determined using the original test set of 253 protein sequences used in Korenberg et al. (1991). The extent to which the PCI result replaced that from SAM depended on the pair of families involved in the classification task, and ranged from 20-80% with an average of 60%.

TABLE 4
Correct Classification Rate for PCI, HMM, and Combination on Large
Test Set

| | | | | |
|---|---|---|---|---|
| | % Correct | % Correct | % Correct | |
| | Globin v Cal- | Globin v | Cal-Bind | Mean % |
| Method | bind | Kinase | v Kinase | Correct |

| | | | | | | | |
|---|---|---|---|---|---|---|---|
| PCI, | 78 | 97 | 77 | 91 | 66 | 68 | 79 |
| SARAH1 | | | | | | | |
| HMM | 100 | 44 | 85 | 82 | 43 | 96 | 75 |
| Combo | 88 | 95 | 82 | 90 | 69 | 70 | 82 |

Parallel cascade identification has a role in protein sequence classification, especially when simple two-way distinctions are useful, or if little training data is available. Binary and multilevel codes were introduced in Korenberg et al. (2000b) so that each amino acid is uniquely represented and equally weighted. The codes enhance classification accuracy by causing greater variability in the numerical profiles for the protein sequences and thus improved inputs for system identification, compared with using Rose scale hydrophobicity values to represent the amino acids. Parallel cascade identification can also be used to locate phosphorylation and ATPase binding sites on proteins, applications readily posed as binary classification problems.

(c) Predicting Whether a Molecule Will Exhibit Biological Activity, e.g., In Drug Discovery, Including the Screening of Databases of Small Molecules to Identify Molecules of Possible Pharmaceutical Use

In “Textual And Chemical Information Processing: Different Domains But Similar Algorithms” (http://www.shef.ac.uk/˜is/publications/infres/paper69.html), Peter Willett at Krebs Institute for Biomolecular Research and Department of Information Studies, University of Sheffield, Sheffield S10 2TN, UK, describes a genetic algorithm for determining whether a molecule is likely to exhibit biological activity. Relevant properties such as molecular weight, the _k (a shape index) and the number of aromatic rings, rotatable bonds, hydrogen-bond donor atoms and hydrogen-bond acceptor atoms. Each property (also called a feature) was represented numerically by a value that was allowed to fall within one of 20 bins allocated for that property. The genetic algorithm was used to calculate a weight for each bin of each property, based on a training set of compounds for which the biological activities are available.

The same approach described in this application for predicting the class of gene expression profiles, or for classifying protein sequences or finding active sites on a protein can be used to determine whether a molecule will possess biological activity. For each compound in the training set, the numerical values for the relevant properties can be appended to form a segment, always following the same order of appending the values. A training input can then be constructed by concatenating the segments. The training output can then be defined to have a value over each segment of the training input that is representative of the biological activity of the compound corresponding to that segment. Parallel cascade identification or another model-building technique can then be used to approximate the input/output relation. Then a query compound can be assessed for biological activity by appending numerical values for the relevant properties, in the same order as used above, to form a segment which can be fed to the identified model. The resulting model output can then be used to classify the query compound as to its biological activity using some test of similarity, such as sign of the output mean (Korenberg et al., 2000a) or the mean-square error ratio (Korenberg et al., 2000b).

(d) Distinguishing Exon From Intron DNA And RNA Sequences, and Determining Their Boundaries

This application is described in Appendix C.

(e) Combining Non-Linear System Identifications Methods with Other Methods

As noted above, the method described by Golub et al. provided strong predictions with 100% accurate results for 29 of 34 samples in a second data set after 28 ALL and AML profiles in a first set were used for training. The remaining 5 samples in the second set were not strongly predicted to be members of the ALL or AML classes. The non-linear method of the present invention may be combined with Golub's method to provide predictions for such samples which do not receive a strong prediction. In particular, Golub's method may first be applied to a sample to be tested. Golub's method will provide weighted votes of a set of informative genes and a prediction strength. Samples that receive a prediction strength below a selected threshold may then be used with the parallel cascade indentification technique model described above to obtain a prediction of the sample's classification.

Additional Embodiments

1. The identified parallel cascade model can be used to generate “intermediate signals” as output by feeding the model each of the segments used to form the training input. These intermediate signals can themselves be regarded as training exemplars, and used to find a new parallel cascade model for distinguishing between the corresponding classes of the intermediate signals. Several iterations of this process can be used. To classify a query sequence, all the parallel cascade models would need to be used in the proper order.

2. Note that while the mean and MSE ratios have been discussed in particular as means for making classification decisions, combinations of these quantities can be used. In addition, in place of using a mean-square test of similarity to each class (as in use of the MSE ratios), analogous tests using absolute values or a higher power than 2 can be employed.

Appendix A: Korenberg et al., 2000a, “Parallel Cascade Identification as a Means for Automatically Classifying Proteing Sequences into Structure/Function Groups”, vol. 82, pp. 15-21

Current methods for automatically classifying protein sequences into structure/function groups, based on their hydrophobicity profiles, have typically required large training sets. The most successful of these methods are based on hidden Markov models, but may require hundreds of exemplars for training in order to obtain consistent results. In this paper, we describe a new approach, based on nonlinear system identification, which appears to require little training data to achieve highly promising results.

1 Introduction

Stochastic modeling of hydrophobicity profiles of protein sequences has led to methods being proposed for automatically classifying the sequences into structure/function groups (Baldi et al. 1994; Krogh et al. 1994; Regelson 1997; Stultz et al. 1993; White et al. 1994). Various linear modeling techniques for protein sequence classification, including a Fourier domain approach (McLachlan 1993), have been suggested but most have not shown impressive performance may in experimental trials (about 70% in two-way classifications). A hidden Markov modeling approach (Baldi et al. 1994; Krogh et al. 1994; Regelson 1997) has been more effective in classifying protein sequences, but its performance may depend on the availability of large training sets and require a lengthy training time. This is because thousands of parameters might be incorporated into each hidden Markov model to obtain reasonable classification accuracy (Baldi et al. 1994; Regelson 1997). Hence, a potential drawback of the hidden Markov modeling approach to classifying proteins is the possible need of using large training sets (i.e., hundreds of exemplars) in order to obtain consistent results (Regelson 1997).

Here, in a pilot study, we propose classifying protein sequences by means of a technique for modeling dynamic nonlinear systems, known as parallel cascade identification (Korenberg 1991), and show that it is capable of highly accurate performance in experimental trials. This method appears to be equally or more discriminating than other techniques (Krogh et al. 1994; McLachlan 1993; Regelson 1997; Stultz et al. 1993; White et al. 1994) when the size of the training sets is limited. Remarkably, when only a single sequence from each of the globin, calcium-binding, and kinase families was used to train the parallel cascade models, an overall two-way classification accuracy of about 79% was obtained in classifying novel sequences from the families.

This paper is addressed to managers of large protein databases to inform them of an emerging methodology for automatic classification of protein sequences. It is believed that the proposed method can usefully be employed to supplement currently used classification techniques, such as those based on hidden Markov models. This is because, as detailed below, the new method appears to require only a few representative protein sequences from families to be distinguished in order to construct effective classifiers. Hence, for each classification task, the accuracy may be enhanced by building numerous classifiers, each trained on different exemplars from the protein families, and have these classifiers vote on new classification decisions (see Discussion). Because the proposed method appears to be effective while differing considerably from that of hidden Markov models, there are likely benefits from employing them together. For example, when the two methods reach the same classification decision, there may well be increased confidence in the result.

It is also hoped that individual scientists involved in various aspects of protein research will be interested in the new approach to automatically classifying protein sequences. For this reason, some detail is provided about the parallel cascade identification method, and also the construction of one particular protein sequence classifier (globin versus calcium-binding) is elaborated upon as an example.

2 Systems and Methods

For managers of large protein databases, discussion of the modest requirements of computer memory and processing time is not crucial. However, the individual researcher who might wish to investigate this approach further may be interested in knowing that the protein sequence classification algorithm was implemented (in Turbo Basic source code) on a 90-MHz Pentium. Only 640 kilo-bytes of local memory (RAM) are actually required for efficient operation. Training times were only a few seconds while subsequent classification of a new protein sequence could be made in a fraction of a second.

Our data consisted of hydrophobicity profiles of sequences from globin, calcium-binding protein, and protein kinase families. The particular mapping of amino acid to hydrophobicity utilized is the “Rose” scale shown in Table 3 of Cornette et al. (1987), and the resulting profiles were not normalized. The protein sequences were taken from the Brookhaven Protein Data Base of known protein structures.

2.1 Algorithm Description

Consider a discrete-time dynamic (i.e., has memory) nonlinear system described as a “black box”, so that the only available information about the system is its input x(i) and output y(i), i=0, . . . ,l. Parallel cascade identification (Korenberg 1991) is a method for constructing an approximation, to an arbitrary degree of accuracy, of the system's input/output relation using a sum of cascaded elements, when the system has a Wiener or Volterra functional expansion. Each cascade path comprises alternating dynamic linear and static nonlinear elements, and the parallel array can be built up one cascade at a time in the following way.

A first cascade of dynamic linear and static nonlinear elements is found to approximate the input/output relation of the nonlinear system to be identified. The residue—i.e., the difference between the system and the cascade outputs—is treated as the output of a new dynamic nonlinear system, and a second cascade is found to approximate the latter system. The new residue is computed, a third cascade can be found to improve the approximation, and so on. These cascades are found in such a way as to drive the input/residue crosscorrelations to zero. It can be shown (Korenberg 1991) that any nonlinear system having a Volterra or Wiener functional expansion (Wiener 1958) can be approximated to an arbitrary degree of accuracy in the mean-square sense by a sum of a sufficient number of the cascades. For generality of approximation, it suffices if each cascade comprises a dynamic linear element followed by a static nonlinearity, and this cascade structure was used in the present work. However, additional alternating dynamic linear and static nonlinear elements could optionally be inserted into any cascade path.

If yₖ(i) denotes the residue after adding the kth cascade, then for k∃1,

yₖ(i)=yₖ₋₁(i)−zₖ(i) (1)

where yₒ(i)=y(i). The parallel cascade output, z(i), will be the sum of the individual cascade outputs zₖ(i). The (discrete) impulse response function of the dynamic linear element beginning each cascade can, optionally, be defined using a first-order (or a slice of a higher-order) crosscorrelation of the input with the latest residue (discrete impulses δ are added at diagonal values when higher-order crosscorrelations are utilized). When constructing the kth cascade, note that the latest residue is yₖ₋₁(i). Thus, the impulse response function hₖ(j), j=0, . . . ,R, for the linear element beginning the kth cascade will have the form

hₖ(j)=φ_{xy}_{ₖ₋₁}(j), (2)

if the first-order input/residue crosscorrelation φ_{xy}_{ₖ₋₁} is used, or

hₖ(j)=φ_{xxy}_{ₖ₋₁}(j,A)±Cδ(j−A), (3)

if the second-order crosscorrelation φ_{xxy}_{ₖ₋₁} is used, and similarly if a higher-order crosscorrelation is instead employed (Korenberg 1991). In (3), the discrete impulse function δ(j−A)=1 if j=A, and equals 0 otherwise. If (3) is used, then A is fixed at one of the values 0, . . . ,R, and C is adjusted to tend to zero as the mean-square of the residue approaches zero. The linear element's output is

$${u}_{k} ( i ) = {\sum }_{j = 0}^{R} {h}_{k} ( j ) x ( i - j ) . \qquad ( 4 )$$

(4)

Next, the static nonlinearity, in the form of a polynomial, can be best-fit, in the least-square sense, to the residue yₖ₋₁(i). If a higher-degree (say, ≧5) polynomial is to be best-fitted, then for increased accuracy scale the linear element so that its output, uₖ(i), which is the input to the polynomial, has unity mean-square. If D is the degree of the polynomial, then the output of the static nonlinearity, and hence the cascade output, has the form

$${z}_{k} ( i ) = {\sum }_{d = 0}^{D} {a}_{k d} {u}_{k}^{d} ( i ) . \qquad ( 5 )$$

(5) The new residue is then calculated from (1). Since the polynomial in (5) was least-square fitted to the residue uₖ₋₁(i), it can readily be shown that the mean-square of the new residue yₖ(i) is

$$\overline{{y}_{k}^{2} ( i )} = \overline{{y}_{k - 1}^{2} ( i )} - \overline{{z}_{k}^{2} ( i )} , \qquad ( 6 )$$

where the bars denote the mean (time average) operation. Thus, adding a further cascade to the model reduces the mean-square of the residue by an amount equal to the mean-square of the cascade output.

In the simple procedure used in the present study, the impulse response of the dynamic linear element beginning each cascade was defined using a slice of a crosscorrelation function, as just described. Alternatively, a nonlinear mean-square error (MSE) minimization technique can be used to best-fit the dynamic linear and static nonlinear elements in a cascade to the residue (Korenberg 1991). Then the new residue is computed, the minimization technique is used again to best-fit another cascade, and so on. This is much faster than using an MSE minimization technique to best-fit all cascades at once. A variety of such minimization techniques, e.g., the Levenberg-Marquardt procedure (Press et al. 1992), are available, and the particular choice of minimization technique is not crucial to the parallel cascade approach. The central idea here is that each cascade can be chosen to minimize the remaining MSE (Korenberg 1991) such that crosscorrelations of the input with the residue are driven to zero. Alternatively, various iterative procedures can be used to successively update the dynamic linear and static nonlinear elements, to increase the reduction in MSE attained by adding the cascade to the model. However, such procedures were not needed in the present study to obtain good results.

A key benefit of the parallel cascade architecture is that all the memory components reside in the dynamic linear elements, while the nonlinearities are localized in static functions. Hence, approximating a dynamic system with higher-order nonlinearities merely requires estimating higher-degree polynomials in the cascades. This is much faster, and numerically more stable than, say, approximating the system with a functional expansion and estimating its higher-order kernels. Nonlinear system identification techniques are finding a variety of interesting applications and, for example, are currently being used to detect deterministic dynamics in experimental time series (Barahona and Poon 1996; Korenberg 1991). However, the connection of nonlinear system identification with classifying protein sequences appears to be entirely new and surprisingly effective, and is achieved as follows.

Suppose that we form an input signal by concatenating one or more representative hydrophobicity profiles from each of two families of protein sequences to be distinguished. We define the corresponding output signal to have a value of −1 over each input segment from the first family, and a value of 1 over each segment from the second. The fictitious nonlinear system which could map the input into the output would function as a classifier. Nothing is known about this nonlinear system save- its input and output, which suggests resorting to a “black box” identification procedure. Moreover, the ability of parallel cascade identification to conveniently approximate dynamic systems with high-order nonlinearities can be crucial for developing an accurate classifier and, in fact, this approach proved to be effective, as is shown next.

3 Implementation

Using the parallel-cascade approach, we wished to investigate how much information about a structure/function family could be carried by one protein sequence in the form of its hydrophobicity profile. Therefore, we selected one protein sequence from the globin family (Brookhaven designation 1 hds, with 572 amino acids), one from the calcium-binding family (Brookhaven designation 1 scp, with 348 amino acids), and one from the general kinase family (Brookhaven designation 1 pfk, with 640 amino acids). These profiles are unusually long since they are multidomain representatives of their respective families, e.g., the average length of globin family proteins is about 175 amino acids. The lengthier profiles were selected to enable construction of sufficiently elaborated parallel cascade models. Alternatively, one could of course concatenate a number of profiles from the same family together, but we were interested in exploring the information content of single profiles.

Only two-way (i.e., binary) classifiers were constructed in the present work; a multistate classifier can readily be realized by an arrangement of binary classifiers. To build a parallel cascade classifier to distinguish between, say, globin and calcium-binding protein families, begin by splicing together the two selected profiles from these families (forming a 920 point training input). Define the corresponding training output to be −1 over the globin portion and 1 over the calcium-binding portion of the input. The system to be constructed is shown in block-diagram form in FIG. 6, and comprises a parallel cascade model followed by an averager. FIG. 7a shows the input and corresponding output used to train the globin versus calcium-binding classifier.

The input output data were used to build the parallel cascade model, but a number of basic parameters had to be chosen. These were the memory length of the dynamic linear element beginning each cascade, the degree of the polynomial which followed, the maximum number of cascades permitted in the model, and a threshold based on a correlation test for deciding whether a cascade's reduction of the MSE justified its addition to the model. These parameters were set by testing the effectiveness of corresponding identified parallel cascade models in classifying sequences from a small verification set.

This set comprised 14 globin, 10 calcium-binding, and 11 kinase sequences, not used to identify the parallel cascade models. It was found that effective models were produced when the memory length was 25 for the linear elements (i.e., their outputs depended on input lags 0, . . . , 24), the degree of the polynomials was 5 for globin versus calcium-binding, and 7 for globin versus kinase or calcium-binding versus kinase classifiers, with 20 cascades per model. A cascade was accepted into the model only if its reduction of the MSE, divided by the mean-square of the previous residue, exceeded a specified threshold divided by the number of output points used to fit the cascade (Korenberg 1991). For globin versus calcium-binding and calcium-binding versus kinase classifiers, this threshold was set at 4 (roughly corresponding to a 95% confidence interval were the residue-independent Gaussian noise), and for the globin versus kinase classifier the threshold was 14. For a chosen memory length of 25, each parallel cascade model would have a settling time of 24, so we excluded from the identification those output points corresponding to the first 24 points of each distinct segment joined to form the input. The choices made for memory length, polynomial degree, and maximum number of cascades ensured that there were fewer variables introduced into a parallel cascade model than output points used to obtain the model. Training times ranged from about 2 s (for a threshold of 4) to about 8 s (for a threshold of 14).

In more detail, consider how the classifier to distinguish globin from calcium-binding sequences was obtained. Using the training input and output of FIG. 7a, we identified a parallel cascade model via the procedure (Korenberg 1991) described above, for assumed values of memory length, polynomial degree, threshold, and maximum number of cascades allowable. We then tested the identified model for its ability to differentiate between globin and calcium-binding sequences from the small verification set. We observed that the obtained models were not good classifiers unless the assumed memory length was at least 25, so this smallest effective value was selected for the memory length. For this choice of memory length, the best globin versus calcium-binding classification resulted when the polynomial degree was 5 and the threshold was 4, or when the polynomial degree was 7 and the threshold was 14. Both these classifiers recognized all 14 globin and 9 of 10 calcium-binding sequences in the verification set. In contrast, for example, the model found for a polynomial degree of 7 and threshold of 4 misclassified one globin and two calcium-binding sequences. Hence, to obtain the simplest effective model, a polynomial degree of 5 and threshold of 4 were chosen. There are two reasons for setting the polynomial degree to be the minimum effective value. First, this reduces the number of parameters introduced into the parallel cascade model. Second, there are less numerical difficulties in fitting lower-degree polynomials. Indeed, extensive testing has shown that when two models perform equally well on a verification set, the model with the lower-degree polynomials usually performs better on a new test set.

It remained to set the maximum number of cascades to be allowed in the model. Since the selected memory length was 25 and polynomial degree was 5, each dynamic linear/static nonlinear cascade path added to the model introduced a further 25+6=31 parameters. As noted earlier, the training input and output each comprised 920 points, and we excluded from the identification those output points corresponding to the first 24 points of each segment joined to form the input. This left 872 output points available for identifying the parallel cascade model, which must exceed the number of (independent) parameters to be introduced. Hence at most 28 cascade paths could be justified, but to allow some redundancy, a maximum of 20 cascades were allowed. This permitted 620 parameters in the model, slightly more than 70% of the number of output points used for the identification. While normally such a high amount of parameters is inappropriate, it could be tolerated here because of the low-noise “experimental” conditions. That is, well-established hydrophobicity profiles were spliced to form the training input, and the training output, defined to have the values −1 and 1, was precisely known as explained above.

In this way, we employed the small verification set to determine values of memory length, polynomial degree, threshold, and maximum number of cascades allowable, for the parallel cascade model to distinguish globin from calcium-binding sequences. Recall that the training input and output of FIG. 7a had been used to identify the model. FIG. 7b shows that the calculated output of the identified model, when stimulated by the training input, indeed tends to be negative over the globin portion of the input, and positive over the calcium-binding portion.

A test hydrophobicity profile input to a parallel cascade model is classified by computing the average of the resulting output post settling time (i.e., commencing the averaging on the 25th point). The sign of this average determines the decision of the binary classifier (see FIG. 6). More sophisticated decision criteria are under active investigation, but were not used to obtain the present results. Over the verification set, the globin versus calcium-binding classifier, as noted, recognized all 14 globin and 9 of the 10 calcium-binding sequences. The globin versus kinase classifier recognized 12 of 14 globin, and 10 of 11 kinase sequences. The calcium-binding versus kinase classifier recognized all 10 calcium-binding and 9 of the 11 kinase sequences. The same binary classifiers were then appraised over a larger test set comprising 150 globin, 46 calcium-binding, and 57 kinase sequences, which did not include the three sequences used to construct the classifiers. The globin versus calcium-binding classifier correctly identified 96% (144) of the globin and about 85% (39) of the calcium-binding hydrophobicity profiles. The globin versus kinase classifier correctly identified about 89% (133) of the globin and 72% (41) of the kinase profiles. The calcium-binding versus kinase classifier correctly identified about 61% (28) of the calcium-binding and 74% (42) of the kinase profiles. Interestingly, a blind test of this classifier had been conducted since five hydrophobicity profiles had originally been placed in the directories for both the calcium-binding and the kinase families. The classifier correctly identified each of these profiles as belonging to the calcium-binding family. Out of the 506 two-way classification decisions made by the parallel cascade models on the test set, 427 (=84%) were correct, but the large number of globin sequences present skews the result. If an equal number of test sequences had been available from each family, the overall two-way classification accuracy expected would be about 79%. The two-way classification accuracies for globin, calcium-binding, and kinase sequences (i.e., the amount correctly identified of each group) were about 92%, 73%, and 73%, respectively. These success rates cannot be directly compared with those reported in the literature (Krogh et al. 1994; Regelson 1997) for the hidden Markov model approach because the latter attained such success rates with vastly more training data than required in our study (see Discussion).

Using 2×2 contingency tables, we computed the chi-square statistic for the classification results of each of the three binary classifiers over the larger test set. The null hypothesis states that the classification criterion used by a parallel cascade model is independent of the classification according to structure/function. For the null hypothesis to be rejected at the 0.001 level of significance requires a chi-square value of 10.8 or larger. The computed chi-square values for the globin versus calcium-binding, globin versus kinase, and calcium-binding versus kinase classifiers are ≈129, ≈75, and 12.497, respectively, indicating high statistical significance.

How does the length of a protein sequence affect its classification? For the 150 test globin sequences, the average length (±the sample standard deviation σ) was 148.3 (±15.1) amino acids. For the globin versus calcium-binding and globin versus kinase classifiers, the average length of a misclassified globin sequence was 108.7 (±36.4) and 152.7 (±24) amino acids, respectively, the average length of correctly classified globin sequences was 150 (±10.7) and 147.8 (±13.5), respectively. The globin versus calcium-binding classifier misclassified only 6 globin sequences, and it is difficult to draw a conclusion from such a small number, while the other classifier misclassified 17 globin sequences. Accordingly, it is not clear that globin sequence length significantly affected classification accuracy.

Protein sequence length did appear to influence calcium-binding classification accuracy. For the 46 test calcium-binding sequences, the average length was 221.2 (±186.8) amino acids. The average length of a misclassified calcium-binding sequence, for the globin versus calcium-binding and calcium-binding versus kinase classifiers, was 499.7 (±294.5) with 7 sequences misclassified, and 376.8 (±218) with 18 misclassified, respectively. The corresponding average lengths of correctly classified calcium-binding sequences were 171.2 (±95.8) and 121.1 (±34.5), respectively, for these classifiers.

Finally, for the 57 test kinase sequences, the average length was 204.7 (±132.5) amino acids. The average length of a misclassified kinase sequence, for globin versus kinase and calcium-binding versus kinase classifiers, was 159.5 (±137.3) with 16 sequences misclassified, and 134.9 (±64.9) with 15 misclassified, respectively. The corresponding average lengths of correctly classified kinase sequences, for these classifiers, were 222.4 (±126.2) and 229.7 (±141.2), respectively.

Thus, sequence length may have affected classification accuracy for calcium-binding and kinase families, with average length of correctly classified sequences being shorter than and longer than, respectively, that of incorrectly classified sequences from the same family. However, neither the correctly classified nor the misclassified sequences of each family could be assumed to come from normally distributed populations, and the number of misclassified sequences was, each time, much less than 30. For these reasons, statistical tests to determine whether differences in mean length of correctly classified versus misclassified sequences are significant will be postponed to a future study with a larger range of sequence data. Nevertheless, the observed differences in means of correctly classified and misclassified sequences, for both calcium-binding and kinase families, suggest that classification accuracy may be enhanced by training with several representatives of these families. Two alternative ways of doing this are discussed in the next section.

4 Discussion

The most effective current approach for protein sequence classification into structure/function groups uses hidden Markov models, a detailed investigation of which was undertaken by Regelson (1997). Some of her experiments utilized hydrophobicity profiles (Rose scale, normalized) from each of which the 128 most significant power components were extracted to represent the corresponding protein sequence. The families to be distinguished, namely globin, calcium-binding, kinase, and a ““random” group drawn from 12 other classes, were represented by over 900 training sequences, with calcium-binding having the smallest number, 116. Successful classification rates on novel test sequences, using trained left-to-right hidden Markov models, ranged over 92-97% for kinase, globin, and “random” classes, and was a little less than 50% for calcium-binding proteins (Table 4.30 in Regelson 1997). These results illustrate that, with sufficiently large training sets, left-to-right hidden Markov models are very well suited to distinguishing between a number of structural/functional classes of protein (Regelson 1997).

It was also clearly demonstrated that the size of the training set strongly influenced generalization to the test set by the hidden Markov models (Regelson 1997). For example, in other of Regelson's experiments, the kinase training set comprised 55 short sequences (from 128-256 amino acids each) represented by transformed property profiles, which included power components from Rose scale hydrophobicity profiles. All of these training sequences could subsequently be recognized, but none of the sequences in the test set (Table 4.23 in Regelson 1997), so that 55 training sequences from one class were still insufficient to achieve class recognition.

The protein sequences in our study are a randomly selected subset of the profiles used by Regelson (1997). The results reported above for parallel cascade classification of protein sequences surpass those attained by various linear modeling techniques described in the literature. A direct comparison with the hidden Markov modeling approach has yet to be done based on the amount of training data used in our study. While three protein sequence hydrophobicity profiles were used to construct the training data for the parallel cascade models, an additional 35 profiles forming our verification set were utilized to gauge the effectiveness of trial values of memory length, polynomial degree, number of cascades, and thresholds. However, useful hidden Markov models might not be trainable on only 38 hydrophobicity profiles in total, and indeed it is clear from Regelson (1997) that several hundred profiles could sometimes be required for training to obtain consistent results.

Therefore, for the amount of training data in our pilot study, parallel cascade classifiers appear to be comparable to other currently available protein sequence classifiers. It remains open how parallel cascade and hidden Markov model performance compare using the large training sets often utilized for the latter approach. However, because the two approaches differ greatly, they may tend to make their classification errors on different sequences, and so might be used together to enhance accuracy.

Several questions and observations are suggested by the results of our pilot study so far. Why does a memory length of 25 appear to be optimal for the classifiers? Considering that hydrophobicity is a major driving force in folding (Dill 1990) and that hydrophobic-hydrophobic interactions may frequently occur between amino acids which are well-separated along the sequence, but nearby topologically, it is not surprising that a relatively long memory may be required to capture this information. It is also known from autoregressive moving average (ARMA) model studies (Sun and Parthasarathy 1994) that hydrophobicity profiles exhibit a high degree of long-range correlation. Further, the apparent dominance of hydrophobicity in the protein folding process probably accounts for the fact that hydrophobicity profiles carry a considerable amount of information regarding a particular structural class. It is also interesting to note that the globin family in particular exhibits a high degree of sequence diversity, yet our parallel cascade models were especially accurate in recognizing members of this family. This suggests that the models developed here are detecting structural information in the hydrophobicity profiles.

In future work, we will construct multi-state classifiers, formed by training with an input of linked hydrophobicity profiles representing, say, three distinct families, and an output which assumes values of, say, −1, 0, and 1 to correspond with the different families represented. This work will consider the full range of sequence data available in the Swiss-Prot sequence data base. We will compare the performance of such multi-state classifiers with those realized by an arrangement of binary classifiers. In addition, we will investigate the improvement in performance afforded by training with an input having a number of representative profiles from each of the families to be distinguished. An alternative strategy to explore is identifying several parallel cascade classifiers, each trained for the same discrimination task, using a different single representative from each family to be distinguished. It can be shown that, if the classifiers do not tend to make the same mistakes, and if each classifier is right most of the time, then the accuracy can be enhanced by having the classifiers vote on each decision. To date, training times have only been a few seconds on a 90-MHz Pentium, so there is some latitude for use of lengthier and more elaborate training inputs, and/or training several classifiers for each task.

The advantage of the proposed approach is that it does not require any a priori knowledge about which features distinguish one protein family from another. However, this might also be a disadvantage because, due to its generality, it is not yet clear how close proteins of different families can be to each other and still be distinguishable by the method. Additional work will investigate, as an example, whether the approach can be used to identify new members of the CIC chloride channel family, and will look for the inevitable limitations of the method. For instance, does it matter if the hydrophobic domains form alpha helices or beta strands? What kinds of sequences are particularly easy or difficult to classify? How does the size of a protein affect its classification? We began an investigation of the latter question in this paper, and it appeared that sequence length was a factor influencing the accuracy of the method in recognizing calcium-binding and kinase proteins, but was less evidently so for globins. This suggested that using further calcium-binding and kinase exemplars of differing lengths in training the parallel cascade classifiers may be especially important to increase classification accuracy.

The present work appears to confirm that hydrophobicity profiles store significant information concerning structure and/or function as was observed by Regelson (1997). Our work also indicates that even a single protein sequence may reveal much about the characteristics of the whole family, and that parallel cascade identification is a particularly efficient means of extracting characteristics which distinguish the families. We are now exploring the use of parallel cascade identification to distinguish between coding (exon) and non-coding (intron) DNA or RNA sequences. Direct applications of this work are both in locating genes and increasing our understanding of how RNA is spliced in making proteins.

Acknowledgements. Supported in part by grants from the Natural Sciences and Engineering Research Council of Canada. We thank the referees for their astute comments on the manuscript.

### REFERENCES

Baldi P, Chauvin Y, Hunkapiller T, McClure, M A (1994) Hidden Markov models of biological primary sequence information. Proc Natl Acad Sci USA 91:1059-1063

Barahona M, Poon C-S (1996) Detection of nonlinear dynamics in short, noisy time series. Nature 381:215-217

Cornette J L, Cease K B, Margalit H, Spouge J L, Berzofsky J A, DeLisi C (1987) Hydrophobicity scales and computational techniques for detecting amphipathic structures in proteins. J Mol Biol 195:659-685

Dill K A (1990) Dominant forces in protein folding. Biochemistry 29:7133-7155

Korenberg M J (1991) Parallel cascade identification and kernel estimation for nonlinear systems. Ann Biomed Eng 19:429-455

Krogh A, Brown M, Mian I S, Sjolander K, Haussler D (1994) Hidden Markov models in computational biology—applications to protein modeling. J Mol Biol 235:1501-1531

McLachlan A D (1993) Multichannel Fourier analysis of patterns in protein sequences. J Phys Chem 97:3000-3006

Press W H, Teukolsky S A, Vetterling W T, Flannery B P (1992) Numerical recipes in C:the art of scientific computing, 2nd edn. Cambridge University Press, Cambridge

Regelson M E (1997) Protein structure/function classification using hidden Markov models. Ph.D. Thesis, The Beckman Institute, California Institute of Technology, Pasadena

Stultz C M, White J V, Smith T F (1993) Structural analysis based on state-space modeling. Protein Sci 2:305-314

Sun S J, Parthasarathy R (1994) Protein-sequence and structure relationship ARMA spectral-analysis—application to membrane-proteins. Biophys J 66:2092-2106

White J V, Stultz C M, Smith T F (1994) Protein classification by stochastic modeling and optimal filtering of amino-acid-sequences. Math Biosci 119:35-75

Wiener N (1958) Nonlinear problems in random theory. MIT Press, Cambridge, Mass

FIG. 6. Use of a parallel cascade model to classify a protein sequence into one of two families. Each L is a dynamic linear element with settling time (i.e., maximum input lag) R, and each N is a static nonlinearity. The protein sequence in the form of a hydrophobicity profile x(i), i=0, . . . ,I, is fed to the parallel cascade model, and the average model output \overline{z} is computed. If \overline{z} is less than zero, the sequence is classified in the first family, and if greater than zero in the second family.

FIG. 7. a The training input and output used to identify the parallel cascade model for distinguishing globin from calcium-binding sequences. The input x(1) was formed by splicing together the hydrophobicity profiles of one representative globin and calcium-binding sequence. The output y(i) was defined to be −1 over the globin portion of the input, and 1 over the calcium-binding portion. b The training output y(i) and the calculated output z(i) of the identified parallel cascade model evoked by the training input of (a). Note that the calculated output tends to be negative (average value: −0.52) over the globin portion of the input, and positive (average value: 0.19) over the calcium-binding portion

Appendix B: Korenberg et al., 2000b “Automatic Classification of Protein Sequences into Structure/Function Groups via Parallel Cascade Identification: A Feasibility Study”, Annals of Biomedical Engineering, vol. 28, pp. 803-811.

A recent paper introduced the approach of using nonlinear system identification as a means for automatically classifying protein sequences into their structure/function families. The particular technique utilized, known as parallel cascade identification (PCI), could train classifiers on a very limited set of exemplars from the protein families to be distinguished and still achieve impressively good two-way classifications. For the nonlinear system classifiers to have numerical inputs, each amino acid in the protein was mapped into a corresponding hydrophobicity value, and the resulting hydrophobicity profile was used in place of the primary amino acid sequence. While the ensuing classification accuracy was gratifying, the use of (Rose scale) hydrophobicity values had some disadvantages. These included representing multiple amino acids by the same value, weighting some amino acids more heavily than others, and covering a narrow numerical range, resulting in a poor input for system identification. This paper introduces binary and multilevel sequence codes to represent amino acids, for use in protein classification. The new binary and multilevel sequences, which are still able to encode information such as hydrophobicity, polarity, and charge, avoid the above disadvantages and increase classification accuracy. Indeed, over a much larger test set than in the original study, parallel cascade models using numerical profiles-constructed with the new codes achieved slightly higher two-way classification rates than did hidden Markov models (HMMs) using the primary amino acid sequences, and combining PCI and HMM approaches increased accuracy.

Automatic classification of protein sequences into their structure/function groups, based either upon primary amino acid sequence, or upon property profiles such as hydrophobicity, polarity, and charge, has attracted increasing attention over the past 15 yr.^{1,8-10,12-14} Proposed approaches to this problem include both linear methods such as Fourier domain analysis₁₀ and variants of hidden Markov modeling^{1,9,12}. The latter approaches have been the most effective at automatically classifying protein sequences, but in some applications¹² have required large training sets (hundreds of exemplars from the families to be distinguished) in order to obtain consistent results. Other times these approaches have performed respectably with very little training data (e.g., see tests below). Certainly, when the training sets are sufficiently large, the hidden Markov model (HMM) approaches^{1,9,12} have yielded very impressive classification results over many families of protein sequences.

In the present paper, we examine a recent approach⁸ based on parallel cascade identification^{5,6} (PCI), which can also be used to classify protein sequences. It is not intended that this approach would ever replace the HMM methods as the preferred classification means for managers of large databases. However, there are at least two areas where the PCI method can be usefully employed. First, it may be an aid to individual scientists engaged in various aspects of protein research. This is because the method can create effective classifiers after training on very few exemplars from the families to be distinguished, particularly when binary (two-way) decisions are required. This can be an advantage, for instance, to researchers who have newly discovered an active site on a protein, have only a few examples of it, and wish to accelerate their search for more by screening novel sequences. Second, as discussed below, the classifiers produced by the approach have the potential of being usefully employed with HMMs to enhance classification accuracy.

The approach in question was introduced in a recent paper⁸ that proposed to use techniques from nonlinear system identification in order to classify protein sequences into their structure/function families. The particular technique employed, called parallel cascade identification,^{5,6} was used to build binary classifiers for distinguishing protein sequences from the globin, calcium-binding, and kinase families. Using a single example of a hydrophobicity profile from each of these families for training, the parallel cascade classifiers were able to achieve two-way classification accuracies averaging about 79% on a test set.⁸

While these findings were highly promising, the investigation was essentially a pilot study with a limited number (253) of test sequences. In addition, the mapping from amino acid to hydrophobicity value was determined according to the Rose scale.³ Because this scale is not one-to-one, its use entails a loss of information about the primary amino acid sequence, with the 20 amino acids being mapped into 14 hydrophobicity values. Moreover, the differing magnitudes of these values cause some amino acids to be weighted more heavily than others. Finally, the values have a narrow range, from 0.52 to 0.91, making the resulting hydrophobicity profiles poor inputs for nonlinear system identification.

The present paper describes a more thorough and rigorous investigation of the performance of parallel cascade classification of protein sequences. In particular, we utilized more than 16,000 globin, calcium-binding, and kinase sequences from the NCBI (National Center for Biotechnology Information, at ncbi.nlm.nih.gov) database to form a much larger set for testing. In order to avoid biasing the present study toward a higher success rate, no attempt was made to search for “better” training sequences, and in fact only the three primary amino acid sequences from the earlier study⁸ were used here for creating the training inputs.

However, instead of relying on hydrophobicity values, the present paper introduces the use of a binary or multilevel numerical sequence to code each amino acid uniquely. The coded sequences are contrived to weight each amino acid equally, and can be assigned to reflect a relative ranking in a property such as hydrophobicity, polarity, or charge. Moreover, codes assigned using different properties can be concatenated, so that each composite coded sequence carries information about the amino acid's rankings in a number of properties.

The codes cause the resulting numerical profiles for the protein sequences to form improved inputs for system identification. As shown below, using the new amino acid codes, parallel cascade classifiers were more accurate (85%) than were hydrophobicity-based classifiers in the earlier study,⁸ and over the large test set achieved correct two-way classification rates averaging 79%. For the same three training exemplars, hidden Markov models using primary amino acid sequences averaged 75% accuracy. We also show that parallel cascade models can be used in combination with hidden Markov models to increase the success rate to 82%.

System and Methods

The protein sequence classification algorithm⁸ was implemented in Turbo Basic on 166 MHz Pentium MMX and 400 MHz Pentium II computers. Due to the manner used to encode the sequence of amino acids, training times were lengthier than when hydrophobicity values were employed, but were generally only a few minutes long, while subsequently a sequence could be classified by a trained model in only a few seconds or less. Compared to hidden Markov models, parallel cascade models trained faster, but required about the same amount of time to classify new sequences.

Sequences from globin, calcium-binding protein, and protein kinase families were converted to numerical profiles using the scheme set out below. The following data sets were used in our study:

(i) The training set, identical to that from the earlier study,⁸ comprised one sequence each from globin, calcium-binding, and general kinase families, having respective Brookhaven designations 1HDS (with 572 amino acids), 1SCP (with 348 amino acids), and 1PFK (with 640 amino acids). This set was used to train a parallel cascade model for distinguishing between each pair of these sequences, as described in the next section.

(ii) The first (original) test set comprised 150 globin, 46 calcium-binding, and 57 kinase sequences, which had been selected at random from the Brookhaven Protein Data Bank (now at rcsb.org) of known protein structures. This set was identical to the test set used in the earlier study.⁸

(iii) The second (large) test set comprised 1016 globin, 1864 calcium-binding, and 13,264 kinase sequences from the NCBI database, all having distinct primary amino acid sequences. The sequences for this test set were chosen exhaustively by keyword search. As explained below, only protein sequences with at least 25 amino acids could be classified by the particular parallel cascade models constructed in the present paper, so this was the minimum length of the sequences in our test sets.

Binary and Multilevel Sequence Representations for Amino Acids

Hydrophobicity profiles constructed according to the Rose scale³ capture significant information concerning structure and/or function, enabling them to be used successfully in protein classification.^{8,12} However, as observed above, the Rose scale is not a one-to-one mapping, so that different amino acid sequences can have identical hydrophobicity profiles. Moreover, the scale covers a narrow range of values, while causing some amino acids to be weighted more heavily than others. These characteristics make the profiles relatively poor inputs for nonlinear system identification. In addition, software is publicly available for using hidden Markov models to classify protein sequences, not by their hydrophobicity profiles, but rather by their primary amino acid sequences, e.g., the well-known sequence alignment and modeling (SAM) system of Hughey and Krogh.⁴

In order to have some comparison of performance when both HMM and PCI approaches receive either the primary amino acid sequences or equivalent information, a new scheme for coding the amino acids was used. The scheme equally weights each amino acid, and causes greater variability in the numerical profiles representing the protein sequences, resulting in better inputs for system identification. At the same time, the relative ranking imposed by the Rose scale can be almost completely preserved. The scheme is similar to, but more elaborate than, one used in recent work on distinguishing exon from intron DNA sequences⁷.

In the latter problem, it was necessary to find a numerical representation for the bases adenine (A), thymine (T), guanine (G), and cytosine (C). In work concerned with efficiently comparing two DNA sequences via crosscorrelation, Cheever et al.² had suggested representing the bases A, T, G, C by, respectively, 1, −1, i, −i, where i=\sqrt{−1}, Directly using these complex numbers to represent the bases in DNA sequences would require two inputs (for the real and imaginary parts). In order to avoid the need for two-input parallel cascade models, this representation was modified,⁷ so that bases A, T, G, C in a sequence were encoded, respectively, by ordered pairs (0, 1), (0, −1), (1, 0), (−1, 0). This doubled the length of the sequence, but allowed use of a single real input. Note that here purines A, G are represented by pairs of the same sign, as are pyrimidines C, T. Provided that this biochemical criterion was met, good classification would result.⁷ Also, many other binary representations were explored, such as those using only ±1 as entries, but it was found that within a given pair, the entries should not change sign.⁷ For example, representing a base by (1, −1) did not result in a good classifier.

The same approach was applied in the present paper to develop numerical codes for representing each of the amino acids. Thus, within the code for an amino acid, the entries should not change sign. To represent the 20 amino acids, each of the codes could have five entries, three of them 0, and the other two both 1 or both −1. There are (₂⁵)=10 such codes of each sign, so the 20 amino acids can be uniquely coded this way. Next, the codes are preferably not randomly assigned to the amino acids, but rather in a manner that adheres to a relevant biochemical property. Consequently, the amino acids were ranked according to the Rose hydrophobicity scale (breaking ties), and then the codes were assigned in descending value according to the binary numbers corresponding to the codes.

The resulting scale in Table 1, where the bottom half is the negative mirror image of the top half, will be referred to as SARAH1 (“simultaneously axially and radially aligned hydrophobicities”). In a similar scale, SARAH2 in Table 2, each code again has five entries, but here two of them are 0, while the other three all equal 1 or all equal −1.

TABLE 1
SARAH1 scale.

| Amino acid | Binary code |
|---|---|
| C | 1,1,0,0,0 |
| F | 1,0,1,0,0 |
| I | 1,0,0,1,0 |
| V | 1,0,0,0,1 |
| L | 0,1,1,0,0 |
| W | 0,1,0,1,0 |
| M | 0,1,0,0,1 |
| H | 0,0,1,1,0 |
| Y | 0,0,1,0,1 |
| A | 0,0,0,1,1 |
| G | 0,0,0,−1,−1 |
| T | 0,0,−1,0,−1 |
| S | 0,0,−1,−1,0 |
| R | 0,−1,0,0,−1 |
| P | 0,−1,0,−1,0 |
| N | 0,−1,−1,0,0 |
| D | −1,0,0,0,−1 |
| Q | −1,0,0,−1,0 |
| E | −1,0,−1,0,0 |
| K | −1,−1,0,0,0 |

TABLE 2
SARAH2 scale.

| Amino acid | Binary code |
|---|---|
| C | 1,1,1,0,0 |
| F | 1,1,0,1,0 |
| I | 1,1,0,0,1 |
| V | 1,0,1,1,0 |
| L | 1,0,1,0,1 |
| W | 1,0,0,1,1 |
| M | 0,1,1,1,0 |
| H | 0,1,1,0,1 |
| Y | 0,1,0,1,1 |
| A | 0,0,1,1,1 |
| G | 0,0,−1,−1,−1 |
| T | 0,−1,0,0−1,−1 |
| S | 0,−1,−1,0,−1 |
| R | 0,−1,−1,−1,0 |
| P | −1,0,0,−1,−1 |
| N | −1,0,−1,0,−1 |
| D | −1,0,−1,−1,0 |
| Q | −1,−1,0,0−1 |
| E | −1,−1,0,−1,0 |
| K | −1,−1,−1,0,0 |

Using one of these scales to encode a protein sequence yields a numerical profile five times longer than the original sequence, but one where each amino acid is uniquely represented and equally weighted. Alternatively, either of the scales can be employed to translate a protein sequence into a profile consisting of five signals, but this approach, which leads to use of five-input parallel cascade models,⁶ will be deferred to a future paper. The greater range covered by the new numerical profiles, compared with the (Rose scale) hydrophobicity profiles, results in improved inputs for training the parallel cascade models, and more accurate classification as shown below.

While the above scales carry information about hydrophobicity, scales can similarly be constructed to imbed other chemical or physical properties of the amino acids such as polarity, charge, α-helical preference, and residue volume. Since each time the same binary codes are assigned to the amino acids, but in an order dependent upon their ranking by a particular property, the relative significance of various factors in the protein folding process can be studied in this way. It is clear that randomly assigning the binary codes to the amino acids does not result in effective parallel cascade classifiers. In addition, the codes can be concatenated to carry information about a number of properties. In this case, the composite code for an amino acid can have 1, −1, and 0 entries, and so can be a multilevel rather than binary representation.

Finally, not only could binary code representations be used here and in the DNA work,⁷ but also, as shown in the next section, analogous combinations of default parameters for training parallel cascade models were employable in the two studies. Moreover, the same criterion for making classification decisions could be utilized in both applications.

Building the Parallel Cascade Classifiers

The application of nonlinear system identification to automatic classification of protein sequences was introduced in the earlier study.⁸ Briefly, we begin by choosing representative sequences from two or more of the families to be distinguished, and represent each sequence by a profile corresponding to a property such as hydrophobicity or to amino acid sequence. Then we splice these profiles together to form a training input, and define the corresponding training output to have a different value over each family or set of families that the classifier is intended to recognize.

For example, consider building a binary classifier intended to distinguish between calcium-binding and kinase families using their numerical profiles constructed according to the SARAH1 scale. The system to be constructed is shown in FIG. 8, and comprises a parallel array of cascades of dynamic linear and static nonlinear elements. We start by splicing together the profiles for the calcium-binding sequence 1 SCP and kinase sequence 1 PFK, to form a 4940 point training input x(i). The input has this length because the 1SCP and 1PFK sequences have 348 and 640 amino acids respectively and, as the SARAH1 scale is used in this paper, each amino acid is replaced with a code five digits long. However, as noted above, the scale could have instead been used to create five signals, each 988 points in length, for a five-input parallel cascade model. No preprocessing of the data is employed. Define the corresponding training output y(i) to be −1 over the calcium-binding, and 1 over the kinase, portions of the input [FIG. 9(a)]. We now seek a dynamic (i.e., has memory) nonlinear system which, when stimulated by the training input, will produce the training output. Clearly, such a system would function as a binary classifier, and at least would be able to distinguish apart the calcium-binding and the kinase representatives.

We can build an approximation to such a dynamic system, given the training input and output, using techniques from nonlinear system identification. The particular technique we have used in this paper, called parallel cascade identification, is more fully explained in Refs. 5 and 6, and is summarized below.

Suppose that x(i), y(i), i=0, . . . ,I, are the training input and output (in this example, I=4939). Then parallel cascade identification is a technique for approximating the dynamic nonlinear system having input x(i) and output y(i) by a sum of cascades of alternating dynamic linear L and static nonlinear N elements.

Previously, a parallel cascade model consisting of a finite sum of dynamic linear, static nonlinear, and dynamic linear (i.e., LNL) cascades was introduced by Palm” to uniformly approximate discrete-time systems that could be approximated by Volterra series. In Palm's parallel LNL model, the static nonlinearities were exponential or logarithmic functions. The dynamic linear elements were allowed to have anticipation as well as memory. While his architecture was an important contribution, Palm¹¹ did not describe any technique for constructing, from input/output data, a parallel cascade approximation for an unknown dynamic nonlinear system.

Subsequently, Korenberg^{5,6} introduced a parallel cascade model in which each cascade comprised a dynamic linear element followed by a polynomial static nonlinearity (FIG. 8). He also provided a procedure for finding such a parallel LN model, given suitable input/output data, to approximate within an arbitrary accuracy in the mean-square sense any discrete-time system having a Wiener¹⁵ functional expansion. While LN cascades sufficed, further alternating L and N elements could optionally be added to the cascades. It was also shown⁶ that any discrete-time finite memory, finite order Volterra series could be exactly represented by a finite sum of these LN cascades, and an upper bound was obtained for the number of required cascade paths, given a Volterra functional of specified memory length and order of nonlinearity.

The parallel cascade identification method^{5,6} can be outlined as follows. A first cascade of dynamic linear and static nonlinear elements is found to approximate the dynamic nonlinear system. The residual, i.e., the difference between the system and the cascade outputs, is calculated, and treated as the output of a new dynamic nonlinear system. A cascade of dynamic linear and static nonlinear elements is now found to approximate the new system, the new residual is computed, and so on. These cascades are found in such a way as to drive the crosscorrelations of the input with the residual to zero. It can be shown that any dynamic nonlinear discrete-time system having a Volterra or a Wiener functional expansion can be approximated, to an arbitrary degree of accuracy in the mean-square sense, by a sum of a sufficient number of the cascades.^{5,6} As mentioned above, for generality of approximation, it suffices if each cascade comprises a dynamic linear element L followed by a static nonlinearity N, and this LN structure was used in the present work, and is assumed in the algorithm description given immediately below.

In more detail, suppose that yₖ(i) denotes the residual after the kth cascade has been added to the model, with y₀(i)=y(i). Let zₖ(i) be the output of the kth cascade. Then, for k=1,2, . . .

yₖ(i)=yₖ₋₁(i)−zₖ(i). (1)

Assume that the output y(i) depends on input values x(i),x(i−1), . . . ,x(i−R), i.e., upon input lags 0, . . . ,R. There are many ways to determine a suitable (discrete) impulse response function hₖ(j), j=0, . . . , R for the linear element L beginning the kth cascade.^{5,6} One practical solution is to set it equal to either the first order crosscorrelation of the input x(i) with the current residual yₖ₋₁(i), or to a slice of a higher order input/residual crosscorrelation, with discrete impulses added or subtracted at diagonal values. Thus, set

hₖ(j)=φ_{xxy}_{ₖ₋₁}(j) (2)

if the first order input residual crosscorrelation φ_{xy}_{ₖ₋₁} is used, or

hₖ(j)=φ_{xxy}_{ₓ₋₁}(j,A)±Cδ(j−A) (3)

if the second order crosscorrelation φ_{xxy}_{ₖ₋₁} is used, and similarly if a higher order crosscorrelation is instead employed. In Eq. (3), the discrete impulse function δ(j−A)=1, j=A, and equals 0 otherwise. If Eq. (3) is used, then A is fixed at one of 0, . . . ,R, the sign of the δ term is chosen at random, and C is adjusted to tend to zero as the mean-square of the residual yₖ₋₁ approaches zero. If the third order input residual crosscorrelation φ_{xxxy}_{ₖ₋₁} were used, then we would have analogously

hₖ(j)=φ_{xxxy}_{ₖ₋₁}(j,A₁,A₂)±C₁δ(j−A₁)±C₂δ(j−A₂), (4)

where A₁,A₂,C₁,C₂, are defined similarly to A,C in Eq. (3).

However, it will be appreciated that many other means can be used to determine the hₖ (j), and that the approach is not limited to use of slices of crosscorrelations. More details of the parallel cascade approach are given in Refs. 5 and 6. Once the impulse response hk(j) has been determined, the linear element's output hₖ(i) can be calculated as

$${u}_{k} ( i ) = {\sum }_{j = 0}^{R} {h}_{k} ( j ) x ( i - j ) . \qquad ( 5 )$$

In Eq. (5), the input lags needed to obtain the linear element's output range from 0 to R, so its memory length is R+1.

The signal uₖ(i) is itself the input to a static nonlinearity in the cascade, which may be represented by a polynomial. Since each of the parallel cascades in the present work comprised a dynamic linear element L followed by a static nonlinearity N, the latter's output is the cascade output

$${z}_{k} ( i ) = {\sum }_{d = 0}^{D} {a}_{kd} {u}_{k}^{d} ( i ) . \qquad ( 6 )$$

The coefficients a_{kd} defining the polynomial static nonlinearity N may be found by best-fitting, in the least-square sense, the output zₖ(i) to the current residual yₖ₋₁(i). Once the kth cascade has been determined, the new residual yₖ(i) can be obtained from Eq. (1), and because the coefficients a_{kd} were obtained by best-fitting, the mean square of the new residual is

$$\overline{{y}_{k}^{2} ( i )} = \overline{{y}_{k - 1}^{2} ( i )} - \overline{{z}_{k}^{2} ( i )} , \qquad ( 7 )$$

where the overbar denotes the mean (time average) operation. Thus, adding a further cascade to the model reduces the mean square of the residual by an amount equal to the mean square of the cascade output.^{5.6} This, of course, by itself does not guarantee that the mean-square error (MSE) of approximation can be made arbitrarily small by adding sufficient cascades. However, the cascades can be created in such a way as to drive the input/residual crosscorrelations arbitrarily close to zero for a sufficient number of cascades. This is the central point that guarantees convergence to the least-square solution, for a given memory length R+1 of the dynamic linear element and polynomial degree D of the static nonlinearity. It implies that in the noise-free case a discrete-time system having a Volterra or a Wiener functional expansion can be approximated arbitrarily accurately by a finite sum of these LN cascades. For a proof of convergence, see Ref. 6.

Once the parallel cascade model has been identified, we calculate its output [FIG. 9(b)] due to the training input, and also the MSE of this output from the training output for calcium-binding and kinase portions of the training input. Recall that the training output has value −1 over the calcium-binding portion, and 1 over the kinase portion, of the training input. Hence we compute a first MSE of the model output from −1 for the calcium-binding portion, and a second MSE from 1 for the kinase portion, of the training input.

The parallel cascade model can now function as a binary classifier as illustrated in FIG. 10, via an MSE ratio test. A sequence to be classified, in the form of its numerical profile x(i) constructed according to the SARAH1 scale, is fed to the model, and we calculate the corresponding output

$$z ( i ) = {\sum }_{k = 1}^{K} {z}_{k} ( i ) , \qquad ( 8 )$$

where K is the number of cascade paths in the final model. Next, we compare the MSE of z(i) from −1, relative to the corresponding MSE for the appropriate training sequence, with the MSE of z(i) from 1, again relative to the MSE for the appropriate training sequence. In more detail, the first ratio is

$${r}_{1} = \frac{\overline{{( z ( i ) + 1 )}^{2}}}{{e}_{1}} , \qquad ( 9 )$$

where e₁ is the MSE of the parallel cascade output from −1 for the training numerical profile corresponding to calcium-binding sequence 1SCP. The second ratio computed is

$${r}_{2} = \frac{\overline{{( z ( i ) - 1 )}^{2}}}{{e}_{2}} , \qquad ( 10 )$$

where e₂ is the MSE of the parallel cascade output from 1 corresponding to kinase sequence 1PFK. In FIG. 10, r₁ and r₂ are referred to as the MSE ratios for calcium binding and kinase, respectively. Each time an MSE is computed, we commence the averaging on the (R+1)th point. If r₁ is less than r₂, the sequence is classified as calcium binding, and if greater, as kinase. This MSE ratio test has also been found to be an effective classification criterion in distinguishing exon from intron DNA sequences.⁷ As noted below, an effective memory length R+1 for our binary classifiers was 125, corresponding to a primary amino acid sequence length of 25, which was therefore the minimum length of the sequences which could be classified by the models identified in the present paper.

Other decision criteria are under active investigation, e.g., computing the distributions of output values corresponding to each training input. Then, to classify a novel sequence, compute the distribution of output values corresponding to that sequence, and choose the training distribution from which it has the highest probability of coming. However, only the MSE ratio criterion just discussed was used to obtain the results in the present paper.

Note that, instead of splicing together only one representative sequence from each family to be distinguished, several representatives from each family can be joined.⁸ It is preferable, when carrying out the identification, to exclude from computation those output points corresponding to the first R points of each segment joined to form the training input.⁸ This is done to avoid introducing error into the identification due to the transition zones where the different segments of the training input are spliced together.

Using this parallel cascade identification and the appropriate training input and output created as described earlier, we found three classifier models, each intended to distinguish between one pair of families. For example, a parallel cascade model was identified to approximate the input/output relation defined by the training data of FIG. 9(a). The three models corresponded to the same assumed values for certain parameters, namely the memory length R+1, the polynomial degree D, the maximum number of cascades permitted in the model, and a threshold for deciding whether a cascade's reduction of the MSE justified its inclusion in the model. To be acceptable, a cascade's reduction of the MSE, divided by the mean square of the current residual, had to exceed the threshold T divided by the number of output points 11 used to estimate the cascade, or equivalently,

$$\overline{{z}_{k}^{2} ( i )} > \frac{T}{{I}_{1}} \overline{{y}_{k - 1}^{2} ( i )} . \qquad ( 11 )$$

This criterion⁶ for selecting candidate cascades was derived from a standard correlation test.

The parallel cascade models were identified using the FIG. 9(a) data, or on corresponding data for training globin versus calcium-binding or globin versus kinase classifiers. Each time we used the same assumed parameter values, the particular combination of which was analogous to that used in the DNA study.⁷ In the latter work, it was found that an effective parallel cascade model for distinguishing exons from introns could be identified when the memory length was 50, the degree of each polynomial was 4, and the threshold was 50, with 9 cascades in the final model. Since in the DNA study the bases are represented by ordered pairs, whereas here the amino acids are coded by 5-tuples, the analogous memory length in the present application is 125. Also, the shortest of the three training inputs here was 4600 points long, compared with 818 points for the DNA study.⁷ Due to the scaling factor of 5/2 reflecting the code length change, a roughly analogous limit here is 20 cascades in the final models for the protein sequence classifiers. In summary, our default parameter values were memory length (R+1) of 125, polynomial degree D of 4, threshold T of 50, and a limit of 20 cascades. FIG. 9(b) shows that when the training input of FIG. 9(a) is fed through the calcium-binding vs kinase classifier, the resulting output is indeed predominately negative over the calcium-binding portion, and positive over the kinase portion, of the input. The next section concerns how the identified parallel cascade models performed over the test sets.

Classification Results for Test Sequences

All results presented here are for two-way classification, based on training with a single exemplar from the globin, calcium-binding, and kinase families.

Original Test Set

The detailed results are shown in Table 3 for the SARAH1 and SARAH2 encoding schemes, with correct classifications by PCI averaging 85% and 86%, respectively. These should be compared with a 79% average success rate in the earlier study8 where Rose scale hydrophobicity values were instead used to represent the amino acids.

TABLE 3
Correct classification rate for PCI on original test set.

| | | | | |
|---|---|---|---|---|
| | % Correct | % Correct | % Correct | Mean |
| Encoding | Globin vs | Globin vs | Cal-Blind vs | % |
| scale | Cal-Bind | Kinase | Kinase | correct |

| | | | | | | | |
|---|---|---|---|---|---|---|---|
| SARAH1 | 84 | 100 | 73 | 100 | 83 | 67 | 85% |
| SARAH2 | 85 | 100 | 79 | 100 | 85 | 67 | 86% |

Large Test Set

The detailed results are shown in Table 4 for PCI using the SARAH1 encoding scheme, for the hidden Markov modeling approach using the SAM system,⁴ and for the combination of SAM with PCI. The SARAH2 scheme was not tested on this set. FIG. 11 shows a flow chart explaining how the combination was implemented. When the HMM probabilities for the two families to be distinguished were very close to each other, the SAM classification was considered to be marginal, and the PCI result was substituted. The cutoff used with SAM was 1 in the NLL-NULL score, which is the negative log of the probability of a match. This cutoff was determined using the original test set of 253 protein sequences. The extent to which the PCI result replaced that from SAM depended on the pair of families involved in the classification task, and ranged from 20% to 80% with an average of 60%.

TABLE 4
Correct classification rate for PCI, HMM, and combination on
large test set.

| | | | | |
|---|---|---|---|---|
| | % Correct | % Correct | % Correct | |
| | Globin vs | Globin vs | Cal-Bind | Mean % |
| Method | Cal-bind | Kinase | vs Kinase | correct |

| | | | | | | | |
|---|---|---|---|---|---|---|---|
| PCI, | 78 | 97 | 77 | 91 | 66 | 68 | 79 |
| SARAH1 | | | | | | | |
| HMM | 100 | 44 | 85 | 82 | 43 | 96 | 75 |
| Combo | 88 | 95 | 82 | 90 | 69 | 70 | 82 |

Conclusion

Parallel cascade identification appears to have a role in protein sequence classification when simple two-way distinctions are useful, particularly if little training data are available. We introduced binary and multilevel codes so that each amino acid is uniquely represented and equally weighted. The codes enhance classification accuracy by causing greater variability in the numerical profiles for the protein sequences and thus improved inputs for system identification, compared with using Rose scale hydrophobicity values to represent the amino acids. We are now exploring the use of parallel cascade identification to locate phosphorylation and ATPase binding sites on proteins, applications readily posed as binary classification problems.

### REFERENCES

¹Baldi, P., Y. Chauvin, T. Hunkapiller, and M. A. McClure. Hidden Markov models of biological primary sequence information. Proc. Natl. Acad. Sci. U.S.A. 91:1059-1063, 1994.

²Cheever, E. A., D. B. Searls, W. Karunaratne, and G. C. Overton. Using signal processing techniques for DNA sequence comparison. Proc. Northeastern Bioengineering Symposium, Boston, Mass., pp. 173-174,1989.

³Cornette, J. L., K. B. Cease, H. Margalit, J. L. Spouge, J. A. Berzofsky, and C. DeLisi. Hydrophobicity scales and computational techniques for detecting amphipathic structures in proteins. J. Mol. Biol. 195:659-685, 1987.

⁴Hughey, R., and A. Krogh. Sequence Alignment and modeling software system. http://www.cse.ucsc.edu/research/compbio/sam.html.

⁵Korenberg, M. J. Statistical identification of parallel cascades of linear and nonlinear systems. Proc. 6th IFAC Symposium on Identification and System Parameter Estimation. 1:580-585, 1982.

⁶Korenberg, M. J. Parallel cascade identification and kernel estimation for nonlinear systems. Ann. Biomed. Eng. 19:429455, 1991.

⁷Korenberg, M. J., E. D. Lipson, and J. E. Solomon. Parallel cascade recognition of exon and intron DNA sequences (unpublished).

⁸Korenberg, M. J., J. E. Solomon, and M. E. Regelson. Parallel cascade identification as a means for automatically classifying protein sequences into structure/function groups. Biol. Cybern. 82:15-21, 2000.

⁹Krogh, A., M. Brown, I. S. Mian, K. Sjolander, and D. Haussler. Hidden Markov models in computational biology—Applications to protein modeling. J. Mol. Biol. 235:1501-1531,1994.

¹⁰McLachlan, A. D. Multichannel Fourier analysis of patterns in protein sequences. J. Phys. Chem. 97:3000-3006, 1993.

¹¹Palm, G. On representation and approximation of nonlinear systems. Part II: Discrete time. Biol. Cybern. 34:49-52, 1979.

¹²Regelson, M. E. Protein structure/function classification using hidden Markov models. Ph.D. Thesis, The Beckman Institute, California Institute of Technology, Pasadena, Calif., 1997.

¹³Stultz, C. M., J. V. White, and T. F. Smith. Structural analysis based on state-space modeling. Protein Sci. 2:305-314, 1993.

¹⁴White, J. V., C. M. Stultz, and T. F. Smith. Protein classification by stochastic modeling and optimal filtering of amino-acid-sequences. Math. Biosci. 119:35-75, 1994.

¹⁵Wiener, N. Nonlinear Problems in Random Theory. Cambridge, Mass.: MIT Press, 1958.

FIG. 8. The parallel cascade model used to classify protein sequences: each L is a dynamic linear element, and each N is a polynomial static nonlinearity.

FIG. 9. (a) The training input x(i) and output y(i) used in identifying the parallel cascade binary classifier intended to distinguish calcium-binding from kinase sequences. The amino acids in the sequences were encoded using the SARAH1 scale in Table 1. The input (dashed line) was formed by splicing together the resulting numerical profiles for one calcium-binding (Brookhaven designation: 1SCP) and one kinase (Brookhaven designation: 1PFK) sequence. The corresponding output (solid line) was defined to be −1 over the calcium-binding and 1 over the kinase portions of the input. (b) The training output y(i) (solid line), and the output z(i) (dashed line) calculated when the identified parallel cascade model was stimulated by the training input of (a). Note that the output z(i) is predominately negative over the calcium-binding, and positive over the kinase, portions of the input.

FIG. 10. Steps for classifying an unknown sequence as either calcium binding or kinase using a trained parallel cascade model. The MSE ratios for calcium binding and kinase are given by Eqs. (9) and (10), respectively.

FIG. 11. Flow chart showing the combination of SAM, which classifies using hidden Markov models, with parallel cascade classification to produce the results in Table 4.

Appendix C: M. J. Korenberg, E. D. Lipson and J. E. Solomon, “Parallel Cascade Recognition of Exon and Intron DNA Sequences”, pages 2-23

Many of the current procedures for detecting coding regions on human DNA sequences actually rely on a combination of a number of individual techniques such as discriminant analysis and neural net methods. A recent paper introduced the notion of using techniques from nonlinear systems identification as one means for classifying protein sequences into their structure/function groups. The particular technique employed, called parallel cascade identification, achieved sufficiently high correct classification rates to suggest it could be usefully combined with current protein classification schemes to enhance overall accuracy. In the present paper, parallel cascade identification is used in a pilot study to distinguish coding (exon) from noncoding (intron) human DNA sequences. Only the first exon, and first intron, sequence with known boundaries in genomic DNA from the β T-cell receptor locus were used for training. Then, the parallel cascade classifiers were able to achieve classification rates of about 89% on novel sequences in a test set, and averaged about 82% when results of a blind test were included. These results indicate that parallel cascade classifiers may be useful components in future coding region detection programs.

Introduction

Automatic methods^{6,11,15,17,18} of interpreting human DNA sequences, such as for detecting coding regions in gene identification, have received increasing attention over the past 10 years. One of the most successful systems, known as GRAIL, uses a neural network for recognizing coding regions on human DNA, which combines a series of coding prediction algorithms¹⁷. Indeed, many systems for coding region detection rely on combining a number of underlying pattern recognition techniques, such as discriminant analysis and neural net methods⁴. In the present paper, we show that a method for nonlinear system modeling, called parallel cascade identification^{7,8}, can provide a further technique for distinguishing coding (exon) from noncoding (intron) DNA sequences, that may combine with existing techniques to enhance accuracy of coding region detection.

In a recent papers¹⁰ parallel cascade identification was used to build classifiers for distinguishing amongst the globin, calcium-binding, and kinase protein families. One advantage of this approach is that it requires very few examples of sequences from the families to be distinguished in order to train effective classifiers. Another advantage is that the approach taken differs significantly from currently used methods, for example those based on hidden Markov modeling.^{1,13} Consequently, parallel cascade classifiers may tend to make their mistakes on different sequences than do hidden Markov models, so that the two approaches might well be combined to enhance classification accuracy. The same potential exists in distinguishing exons from introns, and was the motivation for the present study, which essentially tests the feasibility of applying parallel cascade identification to the latter problem.

The sequences we utilized were from the human β T-cell receptor locus, as published by Rowen, Koop, and Hood.¹⁴ Only those exons and introns were used which (a) had precisely known boundaries and (b) were located on the same strand. No attempt was made to select favorable sequences for training the parallel cascade classifier. Rather, the first intron and exon on the strand were used to train several parallel cascade models that corresponded to trial values of certain parameters such as memory length and degree of nonlinearity. Next, some succeeding introns and exons were used as an evaluation set to select the best one of the candidate parallel cascade classifiers. The selected classifier was then assessed on subsequent, novel introns and exons in a test set to measure its correct classification rate. Lastly, a blind test was carried out on a final “unknown” set of introns and exons.

As shown below, the parallel cascade model trained on the first exon and intron attained correct classification rates of about 89% over the test set. The model averaged about 82% over all novel sequences in the test and “unknown” sets, even though the sequences therein were located at a distance of many introns and exons away from the training pair. These results compare favorably with those reported in the literature^{4,5}, and suggest that parallel cascade classifiers may be employed in combination with currently used techniques to enhance detection of coding regions.

System and Methods

The exon intron differentiation algorithm used the same program to train the parallel cascade classifiers as for protein classification^{9,10}, and was implemented in Turbo Basic on a 166 MHz Pentium MMX. Training times depended on the manner used to encode the sequence of nucleotide bases, but were generally only a few minutes long, while subsequent recognition of coding or noncoding regions required only a few seconds or less. Two numbering schemes were utilized to represent the bases, based on an adaptation of a strategy employed by Cheever et al.²

The latter authors² used a complex number representation for the bases, with the complementary pair Adenine (A) and Thymine (T) represented by 1 and −1 respectively, and complementary pairs Guanine (G) and Cytosine (C) by i and −i respectively, where i=\sqrt{−1}. This requires use of two signals to represent a DNA sequence, one for the real and one for the imaginary parts of the representation.

The need for two signals was avoided in the present paper by adapting the above scheme as follows. Bases A, T, G, and C were replaced with the pairs (0, 1), (0, −1), (1, 0), and (−1, 0) (the brackets are for clarity of display and were not actually used). The pairs could be assigned to the bases in the order given here, but as discussed below, a number of alternative assignments were about equally effective. The use of number pairs doubled the length of each sequence, but allowed representation by one real signal. While the scheme was effective, a disadvantage was that, given only a segment of the resulting signal, the start of each number pair might not always be clear.

Accordingly, a modification was used in which the number pairs were replaced with the corresponding triplets (0, 1, 0.1), (0, −1, −0.1), (1, 0, 0.1), and (−1, 0, −0.1) for bases A, T, G, and C. This way, the endpoint of each triplet was always clear; however as shown below, such a representation produced very similar results to those from using number pairs. Of course, the bases could have been assigned corresponding numbers such as 2, 1, −1, −2, but this would attribute unequal magnitudes to them, and indeed the latter representation proved less effective in trials.

No preprocessing of the sequences was carried out, such as screening for interspersed and simple repeats. This has been recommended in the literature⁴ as a first step to precede all other analysis since these repeats rarely overlap coding portions of exons. Nor was any attempt made to select the sequences for the present study according to their length, although sequence length has been found to be a major factor affecting which programs for gene identification can be used.⁴ The aim was to investigate the ability of parallel cascade classifiers to distinguish exons from introns in isolation from any other recommended procedures or considerations.

Four non-overlapping sets of data were used in the present study:

(i) The training set comprised the first precisely determined intron (117 nucleotides in length) and exon (292 nucleotides in length) on the strand. This intron/exon pair was used to train several candidate parallel cascade models for distinguishing between the two families.

(ii) The evaluation set comprised the succeeding 25 introns and 28 exons with precisely determined boundaries. The introns ranged in length from 88 to 150 nucleotides, with mean length 109.4 and standard deviation 17.4. For the exons, the range was 49 to 298, with mean 277.4 and standard deviation 63.5. This set was used to select the best one of the candidate parallel cascade models.

(iii) The test set consisted of the succeeding 30 introns and 32 exons whose boundaries had been precisely determined. These introns ranged from 86 to 391 nucleotides in length, with mean 134.6 and standard deviation 70.4. The exon range was 49 to 304 nucleotides, with mean 280.9 and standard deviation 59.8. This set was used to measure the correct classification rate achieved by the selected parallel cascade model.

(iv) The “unknown” set comprised 78 sequences, all labeled exon for purposes of a blind test, though some sequences were in reality introns.

They ranged in length from 18 (two sequences) to 1059 nucleotides, with mean 331.4 and standard deviation 293.5. For reasons explained in the next section, a minimum length of 23-24 nucleotides was required for sequences to be classified by the selected parallel cascade model constructed in the present paper, so only the two shortest sequences had to be excluded. The three shortest remaining sequences had lengths of 38, 49, and 50 nucleotides.

Determining the Parallel Cascade Classifiers

Parallel dynamic linear, static nonlinear, dynamic linear cascade models were introduced by Palm¹² in 1979 to uniformly approximate discrete-time systems approximatable by Volterra series. It is of interest to note that in Palm's model, the static nonlinearities were exponential or logarithmic functions. Palm¹² did not suggest a procedure for identifying the model, but his elegant paper motivated much future work. Subsequently, Korenberg^{7,8} showed that, for generality of approximation, it sufficed if each cascade comprised a dynamic linear element followed by a static nonlinearity, provided that the static nonlinear elements were polynomials, and provided a general identification procedure for obtaining the model.

In the present paper, the parallel cascade models for distinguishing exons from introns were obtained by the same steps as for the protein sequence classifiers in the earlier studies.^{9,10} Briefly, we begin by converting each available sequence from the families to be distinguished into a numerical profile. In the case of protein sequences, a property such as hydrophobicity, polarity or charge might be used to map each amino acid into a corresponding value, which may not be unique to the amino acid (the Rose scale³ maps the 20 amino acids into 14 hydrophobicity values). In the case of a DNA sequence, the bases can be encoded using the number pairs or triplets described in the previous section. Next, we form a training input by splicing together one or more representative profiles from each family to be distinguished. Define the corresponding training output to have a different value over each family, or set of families, which the parallel cascade model is to distinguish from the remaining families.

For example, when the number pairs set out above represented the bases A, T, G, and C, the numerical profiles for the first intron and exon, which were used for training, comprised 234 and 584 points respectively (twice the numbers of corresponding nucleotides). Splicing the two profiles together to form the training input x(i), we specify the corresponding output y(i) to be −1 over the intron portion, and 1 over the exon portion, of the input (FIG. 12a). Parallel cascade identification was then used to create a model with approximately the input/output relation defined by the given x(i), y(i) data. Clearly such a model-would act as an intron/exon classifier, or at least be able to distinguish between the two training exemplars. An approach to creating such a parallel cascade model is described in refs. 7, 8, and was used to obtain both the protein sequence classifiers in the earlier studies^{9,10}, and the intron/exon classifiers in the present work. Since the same algorithm was utilized to create the classifiers in each case, only a brief description is given below; and the interested reader is referred to the earlier publications for more details.

The type of solution required is known as “black box” identification, because we seek to identify a dynamic (i.e., has memory) nonlinear system of unknown structure, defined only by its input x(i) and output y(i), i=0, . . . ,I A simple strategy^{7,8} is to begin by finding a first cascade of alternating dynamic linear (L) and static nonlinear (N) elements to approximate the given input output relation. The residue, i.e., the difference between the outputs of the dynamic nonlinear system and the first cascade, is treated as the output of a new nonlinear system. A second cascade of alternating dynamic linear and static nonlinear elements is found to approximate the latter system, and the new residue is computed. A third cascade can be found to improve the approximation, and so on.

The dynamic linear elements in the cascades can be determined in a number of ways, e.g., using crosscorrelations of the input with the latest residue while, as noted above, the static nonlinearities can conveniently be represented by polynomials.^{7,8} The particular means by which the cascade elements are found is not crucial to the approach. However these elements are determined, a central point is that the resulting cascades are such as to drive the input/residue crosscorrelations to zero.^{7,8} Then under noise-free conditions, provided that the dynamic nonlinear system to be identified has a Volterra or a Wiener¹⁶ functional expansion, it can be approximated arbitrarily accurately in the mean-square sense by a sum of a sufficient number of the cascades.^{7,8}

As noted above, for generality of approximation, it suffices if each cascade comprises a dynamic linear element followed by a static nonlinearity, and this LN cascade structure was employed in the present work. However, it will be appreciated that additional alternating dynamic linear and static nonlinear elements could optionally be inserted in any path.^{7,8}

In order to identify a parallel cascade model, a number of basic parameters first have to be specified:

1. the memory length of the dynamic linear element beginning each cascade;

2. the degree of the polynomial static nonlinearity which follows the linear element;

3. the maximum number of cascades allowable in the model; and

4. a threshold based on a standard correlation test for determining whether a cascade's reduction of the mean-square error (mse) justified its addition to the model. A cascade was accepted provided that its reduction of the mse, divided by the mean-square of the current residue, exceeded the threshold divided by the number of output points used in the identification.

These parameters were set by identifying candidate parallel cascade models corresponding to assumed values of the parameters and comparing their effectiveness in distinguishing introns from exons over the evaluation set. In the case of parallel cascade classifiers for protein sequences, a memory length of 25 for each linear element, and a polynomial degree of 5-7 for the static nonlinearities, were utilized.¹⁰ A 25 point memory means that the output of the linear element depends on the input at lags of 0, . . . ,24. Since representation of each base by a number pair doubled input length, a memory length of 50 was initially tried for the linear elements. The first trial value for polynomial degree was 5.

For these choices of memory length and polynomial degree, each LN cascade added to the model introduced 56 further variables. The training input and output each comprised 818 points. However, for a memory length of 50, the parallel cascade model would have a settling time of 49, so we excluded from the identification the first 49 output points corresponding to each segment joined to form the input. This left 720 output points available for identifying the parallel cascade model, which must exceed the total number of variables introduced in the model. To allow some redundancy, a maximum of 12 cascades was allowed. This permitted up to 672 variables in the model, about 93% of the number of output data points used in the identification. While such a large number of variables is normally excessive, there was more latitude here because of the “noise free” experimental conditions. That is, the DNA sequences used to create the training input were precisely known, and so was the training output, defined to have value −1 for the intron portion, and 1 for the exon portion, of the input as described above.

Once a parallel cascade model was identified for assumed values of the basic parameters listed above, its effectiveness is gauged over the evaluation set. A DNA sequence to be classified, in the form of its numerical profile, is fed to the parallel cascade model, and the corresponding output z(i) is computed. The classification decision is made using an mse ratio test.⁹ Thus, the ratio of the mse of z(i) from −1, relative to the corresponding mse for the training intron profile, is compared with the ratio of the mse of z(i) from 1, relative to the mse for the training exon profile. If the first ratio is smaller, then the sequence is classified as an intron; otherwise it is classified as an exon. In computing the mse values for z(i) and for the training profiles, the averaging begins after the parallel cascade model has “settled”. That is, if R+1 is the memory of the model, so that its output depends on input lags 0, . . . ,R, then the averaging to compute each mse commences on the (R+1)-th point. This assumes that the numerical profile corresponding to the DNA sequence is at least as long as the memory of the parallel cascade model. As discussed in the next section, when the number pairs were used to represent the bases, a memory length of 46-48 proved effective. This means that a DNA sequence must be at least 23-24 nucleotides long to be classifiable by the selected parallel cascade model constructed in the present paper.

Results for the Evaluation and Test Sets

When the evaluation set was used to judge candidate classifiers, it was found that an effective parallel cascade model could be trained on the first available intron and exon if the memory length R+1 was 46, and polynomial degree was 5. Using a threshold of 50 resulted in the maximum number of 12 cascade paths being chosen out of 1000 candidates. The %mse of the identified model, defined to be the mse divided by the variance of y(i), times 100%, was 33.3%. Recall that the training input x(i) and output y(i) of FIG. 12a were used to identify the model. FIG. 12b shows that when the training input is fed through the identified model, the calculated output z(i) indeed tends to be negative over the intron portion, and positive over the exon portion, of the input. Moreover, the model correctly classified 22 of the 25 introns, and all 28 exons, in the evaluation set, and based on this performance the classifier was selected to measure its correct classification rate on the novel sequences in the test and “unknown” sets.

Over the test set, the model identified 25 (83%) of the 30 introns and 30 (94%) of the 32 exons, for an average of 89%. In the blind test over the “unknown” set, the model recognized 28 (72%) of 39 introns and 29 (78%) of 37 exons, a 75% average. Thus over the test and “unknown” sets, the correct classifications averaged 82%.

Once the results for the “unknown” set were available, any further tests on this set would not be blind. The “unknown” set was therefore pooled with the test set; this larger test set was then used to show that the correct classification rate was relatively insensitive to a decrease in the degree of the polynomial static nonlinearities utilized in the parallel cascade models.

For example, when the polynomial degree was decreased to 4, but all other parameter settings were left unchanged, 9 cascades were selected for the final model, leaving a %mse of 42.1% over the training intron and exon. The model performed identically to the higher degree polynomial model over the evaluation set, and the correct classification rate over the larger test set was only slightly better than it was for the latter model.

Decreasing the polynomial degree to 3 caused the resulting model to be more accurate over the evaluation set but, as discussed later, the performance did not improve significantly over the larger test set. Thus, it was discovered that “better” classifiers, as gauged over the evaluation set, could be obtained for memory length 46 when the polynomial degree was 3, if the threshold was set at 30, which resulted in only 2 cascade paths being selected out of 1000 candidates. The %mse rose to 73.2%, and as shown in FIG. 12c, the model output z(i) for the training input strays much further from the “ideal” output y(i). The classification ability appeared to improve: now all 25 introns, and 27 of 28 exons, in the evaluation set are recognized.

While effective classifiers had been obtained, it was important to establish that the success was not a fortuitous result of how the number pairs had been assigned to the bases A, T, G, and C. Complementary bases had been represented by number pairs that were the “negative” of each other; e.g., A=(0, 1) and T=(0, −1). Within this limitation, there are 8 ways that the pairs (0, 1), (0, −1), (1, 0), and (−1, 0) can be assigned to bases A, T, G, and C. For each of the 8 representations, the correct classification rate over the evaluation set was determined, when the memory length was set at 46, the polynomial degree at 3, the threshold at 30, and a maximum of 12 cascades were permitted in the final model.

A biochemical criterion was found for different representations to be almost equally effective: namely, the number pairs for the purine bases A and G had to have the same “sign”, which of course meant that the pairs for the pyrimidine bases C and T must also be of same sign. That is, either the pairs (1, 0) and (0, 1) were assigned to A and G in arbitrary order, or the pairs (−1, 0) and (0, −1), but it was not effective for A and G to be assigned pairs (−1, 0) and (0, 1), or pairs (1, 0) and (0, −1). In fact, the limitation to number pairs of same sign for A and G was the only important restriction. Number pairs for complementary bases did not have to be negatives of each other; for example, setting A=(0, 1), G=(1, 0), T=(−1, 0), and C=(0, −1) resulted in an effective classifier over the evaluation set.

While a memory length of 46 had proved effective, we had expected that the best memory length would be a multiple of 6, because each codon is a sequence of three nucleotides, and each base is represented by a number pair. Therefore, for various assignments of the number pairs to the bases, we investigated whether a memory length of 48 was preferable. It was found that, with the number pair assignment set out at the end of the last paragraph, the model fit over the training data improved significantly when the memory length was increased from 46 to 48. Indeed, the mse then fell to 65.9%, which was the lowest value for any of the number pair assignments tested when the memory length was 48. Again, the polynomial degree for each static nonlinearity was 3, and the threshold was 30, which resulted in four cascades being selected out of 1000 candidates. When the resulting parallel cascade classifier was appraised over the evaluation set, it recognized all 25 introns, and 27 of 28 exons, equaling the performance reported above for another classifier. However, the correct classification rate over the larger test set was about 82%, no better than it was for the models with higher degree polynomial nonlinearities.

Finally, we investigated whether following a number pair with a small value such as ±0.1, to indicate the endpoint of each base representation, affected classification accuracy. With A=(0, 1, 0.1), T=(0, −1, −0.1), G=(1, 0, 0.1), and C=(−1, 0, −0.1), a memory length of 72 was utilized to correspond to the representation by triplets rather than pairs, and the polynomial degree and threshold were again set at 3 and 30 respectively. Two cascades were selected out of 1000 candidates, leaving a %mse of 72.9%. The resulting classifier recognized all 25 introns, and 26 of 28 exons, in the evaluation set. This performance does not deviate significantly from that observed above for the use of number pair representations. However, an exhaustive evaluation over every possible assignment of the number triplets to the bases has yet to be carried out.

Discussion

This paper describes a pilot study meant to test the feasibility of using parallel cascade identification as a means for automatically distinguishing exon from intron DNA sequences. To achieve the results reported above, only the first precisely determined intron and exon available were utilized to form the input for training the parallel cascade models. The succeeding 25 introns and 28 exons with known boundaries were used as an evaluation set to discover the optimal values for four parameters that had to be preset before each model could be identified. The selected parallel cascade model attained a correct classification rate averaging about 82% on novel sequences from a test set and an “unknown” set used in a blind test.

A number of possibilities exist for improving these results and will be explored in a future paper. First, since only a single exemplar of an intron and exon was utilized to form the training input above, we will investigate whether any increase in classification accuracy ensues from training with multiple examples of these sequences. One way of doing this is to concatenate a number of intron and exon sequences together to form the training input, with the corresponding output defined to be −1 over each intron portion, and 1 over each exon portion, of the input. Another alternative is to construct several parallel cascade classifiers, each trained using different exemplars of introns and exons, and have the classifiers vote on each classification. It can be shown that provided the classifiers do not tend to make the same mistakes, and are correct most of the time, then accuracy will be enhanced by a voting scheme.

Second, the above results were attained using only parallel cascade identification^{7,8}, rather than a combination of several methods as is the preferred practice⁴. For example, one study⁵ used four coding measures, which were combined in a linear discriminant in order to distinguish coding from noncoding sequences. Half the successive 108 base pair windows in GenBank that were either fully coding or fully noncoding was used to determine parameter values for the coding measures. The other half was used to measure the correct classification rate of the resulting discriminant, which was found⁵to be 88%.

The selected parallel cascade classifier in the present paper, operating alone, was about 89% correct over the test set, and about 82% over all novel sequences from the test and “unknown” sets. Thus, it appears from the present pilot project that parallel cascade identification merits consideration as one further component in a coding recognition scheme. Future work will investigate whether the different direction taken by building parallel cascade classifiers results in a tendency to make different classification errors from other methods, and if so, whether such a tendency can be exploited to create a new, more accurate combination of approaches.

### REFERENCES

### All of which are Incorporated herein by These References

1. Baldi, P., Y. Chauvin, T. Hunkapiller, and M. A. McClure. Hidden Markov models of biological primary sequence information. Proc. Natl. Acad. Sci. USA. 91: 1059-1063,1994.

2. Cheever, E. A., D. B. Searls, W. Karunaratne, and G. C. Overton. Using signal processing techniques for DNA sequence comparison. Proc. Northeastern Bioengineering Symp., Boston, Mass., pp. 173-174, 1989.

3. Cornette, J. L., K. B. Cease, H. Margalit, J. L. Spouge, J. A. Berzofsky, and C. DeLisi. Hydrophobicity scales and computational techniques for detecting amphipathic structures in proteins. J. Mol. Biol. 195: 659-685, 1987.

4. Fickett, J. W. Predictive methods using nucleotide sequences. In: Bioinformatics: a practical guide to the analysis of genes and proteins, edited by A. D. Baxevanis and B. F. F. Ouellette. New York, N.Y.: Wiley, 1998, pp. 231-245.

5. Fickett, J. W. and C.-S. Tung. Assessment of protein coding measures. Nucl. Acids Res. 20: 6441-6450,1992.

6. Gelfand, M. S., A. A. Mironov, and P. A. Pevzner. Gene recognition via spliced alignment. Proc. Natl. Acad. Sci. U.S.A. 93: 9061-9066, 1996.

7. Korenberg, M. J. Statistical identification of parallel cascades of linear and nonlinear systems. IFAC Symp. Ident. Sys. Param. Est. 1: 580-585, 1982.

8. Korenberg, M. J. Parallel cascade identification and kernel estimation for nonlinear systems. Ann. Biomed. Eng. 19: 429-455, 1991.

9. Korenberg, R. David, I. W. Hunter, and J. E. Solomon. Automatic classification of protein sequences into structure/function groups via parallel cascade identification. Submitted for publication.

10. Korenberg, M. J., J. E. Solomon, and M. E. Regelson. Parallel cascade identification as a means for automatically classifying protein sequences into structure/function groups. Biol. Cybern. (in press)

11. Milanesi, L., N. A. Kolchanov, I. B. Rogozin, I. V. Ischenko, A. E. Kel, Y. L. Orlov, M. P. Ponomarenko, and P. Vezzoni. GenView: A computing tool for protein-coding regions prediction in nucleotide sequences. In: Proc. Of the Second International Conference on Bioinformatics, Supercomputing and Complex Genome Analysis, edited by H. A. Lim, J. W. Fickett, C. R. Cantor, and R. J. Robbins. Singapore: World Scientific Publishing, 1993, pp. 573-588.

12. Palm, G. On representation and approximation of nonlinear systems. Part II: Discrete time. Biol. Cybern. 34: 49-52, 1979.

13. Regelson, M. E. Protein structure/function classification using hidden Markov models. Ph.D. Thesis, The Beckman Institute, California Institute of Technology, Pasadena, Calif., 1997.

14. Rowen, L., B. F. Koop, and L. Hood. The complete 685-kilobase DNA sequence of the human beta T cell receptor locus. Science 272 (5269): 1755-1762,1996.

15. Snyder, E. E. and G. D. Stormo. Identifying genes in genomic DNA sequences. In: DNA and Protein Sequence Analysis: A Practical Approach, edited by M. J. Bishop and C. J. Rawlings. Oxford: IRL Press, 1996, pp. 209-224.

16. Wiener, N. Nonlinear Problems in Random Theory. Cambridge, Mass.: MIT Press, 1958.

17. Xu, Y., J. R. Einstein, R. J. Mural, M. Shah, and E. C. Uberbacher. An improved system for exon recognition and gene modeling in human DNA sequences. In: Proceedings of the Second International Conference on Intelligent Systems for Molecular Biology, edited by R. Altman, D. Brutlag, P. Karp, R. Lathrop, and D. Searls. Menlo Park, Calif.: AAAI Press, 1994, pp. 376-383.

18. Zhang, M. Q. Identification of protein coding regions in the human genome based on quadratic discriminant analysis. Proc. Natl. Acad. Sci. U.S.A. 94: 565-568,1997.

### Figure Legend

FIG. 12:

(a) The training input x(i) (dotted line) and output y(i) (solid line) utilized in identifying the parallel cascade model intended to distinguish exon from intron sequences. The input was formed by splicing together the numerical profiles for the first precisely known intron and exon sequence on the strand, with the bases A, T, G, C represented respectively by (0, 1), (0, −1), (1, 0), (−1, 0). The corresponding output was defined to be −1 over the intron and 1 over the exon portions of the input.

(b) The training output y(i) (solid line), and the output z(i) (dotted line) calculated when the training input in (a) stimulated the identified parallel cascade model having 5^{th} degree polynomial static nonlinearities. Note that the output z(i) is predominately negative over the intron, and positive over the exon, portions of the input.

(c) The training output y(i) (solid line), and the new output z(i) (dotted line) calculated when the training input in (a) stimulated the identified parallel cascade model having 3^{rd} degree polynomial static nonlinearities. Although the output z(i) is not as predominately negative over the intron portion as in (b), its greater variance over this portion is used in the classification decision criterion to successfully distinguish novel exons from introns.

[image]

[image]

I claim:

1. A method of predicting whether an unclassified sample of biological information corresponds to a first class or to a second class, comprising:
(a) selecting a first known sample of biologic information, wherein the first sample corresponds to the first class;
(b) selecting a second known sample of biologic information, wherein the second sample correspond to the second class;
(c) selecting a first representative segment from said first sample;
(d) selecting a second representative segment from said second sample;
(e) combining the first and second representative segments to form a training input;
(f) defining an input/output relationship between the training input and a training output, wherein the training output has a first value over said first representative segment and a second value of said second representative segment;
(g) calculating a non-linear system to approximate the input/output relationship; and
(h) applying said non-linear system to said unclassified sample to calculate a set of output values.
